(19) United States
(12) Patent Application Publication
(10) Pub. No.: US 2025/0032638 A1
(43) Pub. Date: Jan. 30, 2025

(54) Novel Cancer Treatments with TLR7/8 Agonists

(71) Applicant: Ascendis Pharma Oncology Division A/S, Hellerup (DK)

(72) Inventors: Vibeke MILLER BREINHOLT, Hellerup (DK); Stina SINGEL, Palo Alto, CA (US)

(21) Appl. No.: 18/716,482

(22) PCT Filed: Dec. 12, 2022
(86) PCT No.: PCT/EP2022/085347

§ 371 Date: Jun. 4, 2024

Related U.S. Application Data
(60) Provisional application No. 63/288,957, filed on Dec. 13, 2021.

(30) Foreign Application Priority Data
Nov. 8, 2022 (EP) 22205987.5

(51) Int. Cl. A61K 47/69; A61J 1/14; A61K 39/395; A61P 35/00

(52) U.S. Cl. CPC A61K 47/6903; A61J 1/14; A61K 39/3955; A61P 35/00

(57) ABSTRACT

The present invention relates to a unit dosage form comprising a therapeutically effective amount of a TLR7/8 agonist conjugate or a pharmaceutically acceptable salt thereof: to a TLR7/8 agonist conjugate for use in the treatment of cancer, wherein the TLR7/8 agonist conjugate is administered via intratumoral administration in a dose ranging from 0.3 mg to 3 mg of TLR7/8 agonist per tumor; and to specific conjugates of resiquimod and related aspects.

The present invention relates to a unit dosage form comprising a therapeutically effective amount of a TLR7/8 agonist conjugate or a pharmaceutically acceptable salt thereof; to a TLR7/8 agonist conjugate for use in the treatment of cancer, wherein the TLR7/8 agonist conjugate is administered via intratumoral administration in a dose ranging from 0.3 mg to 3 mg of TLR7/8 agonist per tumor; and to specific conjugates of resiquimod and related aspects.

Toll-like receptor (TLR) agonists can elicit anti-tumor activity by activating innate immune cells and promoting a proinflammatory microenvironment (Aznar et al., J Immunol. 2017 Jan. 1; 198 (1): 31-39; Marabelle et al., Ann Oncol. 2017 Dec. 1; 28 (suppl_12): xii33-xii43; Hamid et al., Oncologist. 2020 March; 25 (3): e423-e438). Local delivery of TLR agonists has shown encouraging preclinical and clinical anti-tumor activity (Hamid et al., Oncologist. 2020 March; 25 (3): e423-e438; et al., Blood. 2015; 126:1452-1461; Clark et al., Am J Clin Dermatol. 2014; 15:197-216).

However, intratumoral (IT) delivery of unmodified TLR agonists such as resiquimod, a TLR7/8 agonist, can lead to rapid diffusion from the tumor, resulting in acute systemic drug exposure and transient but high levels of peripheral proinflammatory cytokines, thus limiting anti-tumor benefit and increasing risk of cytokine-driven adverse effects.

Similarly, in clinical studies of resiquimod administered orally, resiquimod at a systemic concentration of about 4 ng/mL was well tolerated, whereas systemic toxicity was observed at higher doses. Side effects were consistent with systemic cytokine induction, including fever, headache, shivering, and lymphopenia (Journal of Hepatology 47 (2007) 174-182)

Thus, there is a need for a more efficacious and safer treatment with TLR agonists and it is an object of the present invention to at least partially overcome the shortcomings of current treatment options with TLR agonists.

In a first aspect the present invention relates to a unit dosage form comprising a therapeutically effective amount of a TLR7/8 agonist conjugate or pharmaceutically acceptable salt thereof, wherein the TLR7/8 agonist conjugate comprises one or more TLR 7/8 agonist moieties reversibly conjugated to a polymeric moiety and wherein the unit dosage form comprises 0.3 mg to 3 mg of TLR7/8 agonist.

In a second aspect the present invention relates to a TLR7/8 agonist conjugate for use in the treatment of cancer, wherein the TLR7/8 agonist conjugate comprises one or more TLR7/8 agonist moieties reversibly conjugated to a polymeric moiety, wherein the cancer is a solid tumor and wherein the TLR7/8 agonist conjugate is administered via intratumoral administration in a dose ranging from 0.3 mg to 3 mg of TLR7/8 agonist per tumor.

Surprisingly, a robust upregulation of biomarkers was observed in 4 patient tumor lesions intratumorally injected with the TLR 7/8 agonist conjugate demonstrating activation of local tumor immunity, across different tumor types. These biomarkers of activation of local and abscopal immunity by TLR7/8 agonist include altered expression of Type-I interferon related genes and altered expression of genes otherwise involved in the TLR7/8 signaling pathway.

Specific biomarkers of activation of local and abscopal immunity by TLR7/8 agonist include upregulation of the genes CXCL10, IFNB1, IRF1, IRF3, IRF4, IRF7, IRF8, STAT1, STAT2 in biopsies of patient tumors after treatment compared to baseline.

In addition, consistent upregulation of biomarkers of activation was also observed in 2 tumor lesions which had not been injected with the TLR 7/8 agonist conjugate but were derived from patients who had undergone intratumoral injection with the TLR 7/8 agonist conjugate in other tumor lesions demonstrating activation of abscopal immunity. Of note, on-treatment biopsies were obtained 7 days post dose, suggesting prolonged immune activation maintained for more than a week after each dose. Furthermore, sustained immune activation is seen with repeat dosing in injected tumors from 3 patients.

Furthermore, following intratumoral injection of the TLR7/8 agonist conjugate into a single tumor lesion at up to 0.5 mg per lesion Cₘₐₓ values were generally below 150 μg/mL and well below levels reported in the literature to be associated with cytokine release syndrome (˜4000 μg/mL). Thus, the risk of systemic side effects is greatly reduced.

Within the present invention the terms are used having the meaning as follows.

As used herein, the term “dose” or “unit dose” refers to the predetermined amount of the drug, such as TLR7/8 agonist, administered at one time to produce a certain degree of biological response in a patient. The dose of a drug is governed by its inherent potency and in this case, it is a therapeutic dose or therapeutic unit dose. It is understood that the unit dosage form of the present invention comprises one dose or one unit dose of the TLR7/8 agonist.

Doses can be administered once or multiple times. Drug can be administered for at least six months, a year, two years, until the cancer is cured or indefinitely. Treatment may start upon diagnosis of the cancer. It is understood that treatment may last until the death of the patient.

If a dose is determined for a particular drug, such as a TLR7/8 agonist conjugate of formula (A-3) or (A-6), then the dose can be used as a guide for other TLR7/8 agonist conjugates, such that the dose or other reversible conjugates is the same by moles of TLR7/8 agonist as that for the TLR7/8 agonist of formula (A-3) or (A-6). Such guidance is particularly useful when the other conjugate releases a TLR7/8 agonist moiety with a release half-life within plus or minus 20% of that of the conjugate of TLR7/8 agonist of formula (A-3) or (A-6).

The treatments and methods of the present invention can also be used for treating a population of patients having cancer. Such a population can include at least 10, 100 or 1000 patients or may represent all patients at a particular institution.

Although clinical trials can be useful for determining doses and dosage regimens de novo, the present methods can also be performed not in the course of a clinical trial.

As used herein, the term “dosage form” refers to the physical form that comprises the active pharmaceutical ingredient in combination with selected additional ingredients or excipients and which is intended to be delivered to sites of action within the body by various routes of drug administration. It also refers to the physical form in which a precise mixture of active pharmaceutical ingredients and excipients are presented to help administration and delivery to the sites of action, achieve rapid onset of action and improve bioavailability. As used herein, the term “unit dosage form” refers to a dosage form configured for a single administration to a patient, i.e. a unit dosage form comprises one dose. For example, a unit dosage form can be a single vial or the container containing an amount of drug suitable for a single administration.

As used herein, the term “dosage regimen” is the combination of dose, and frequency with which a drug is administered. Dosage regimen can also include a route of administration (e.g., intratumoral) and/or duration of administration.

As used herein, the terms “treating” and “treatment” refer to curing, reducing or inhibiting further deterioration of at least one sign or symptom of a disease or stabilizing at least one sign or symptom of disease and can be determined by comparing sign(s) and symptom(s) in an individual patient before (baseline) and after receiving treatment or by comparing a population of treated patients to a control population as in a clinical trial or trial with an animal model.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

As used herein the terms “immune checkpoint inhibitor” and “immune checkpoint antagonist” are used synonymously and refer to compounds that interfere with the function of, or inhibit binding of ligands that induce signaling through, cell-membrane expressed receptors that inhibit inflammatory immune cell function upon receptor activation. Such compounds may for example be biologics, such as antibodies, nanobodies, probodies, anticalins or cyclic peptides, or small molecule inhibitors.

As used herein the term “T cells” refers to a type of immune cell that plays a central role in the adaptive immune response. T cells are distinguished from other immune cells by the presence of either an αβ or γδ T cell receptor (TCR) on their cell surface. T cells also express CD3—a protein complex critical for TCR signaling. αβ T cells can be divided into either CD4, CD8, or CD4/CD8 double negative subsets. Due to the high surface density of CD4 and CD8 on CD4 and CD8 T cells, CD4 and CD8 alone can often be used to identify CD4 and CD8 T cells respectively. Following activation via TCR recognition of cognate antigen presented by MHC molecules, T cells can mature and divide to generate effector or memory T cells. Memory T cells are a subset of T cells that have previously encountered and responded to their cognate antigen. Such T cells can recognize pathogenic antigens, such as antigens derived from bacteria or viruses, as well as cancer-associated antigens. T cells can be identified by a person skilled in the art by using phenotypic techniques such as flow cytometry. Phenotypic markers used to identify T cells are generally conserved in mammals and include CD3, TCRα, TCRβ, TCRδ, CD4, and CD8. Phenotypic markers used to identify memory T cells can vary by species and by tissue, but may include cell surface markers such as CD45RO, LY6C, CD44, and CD95.

As used herein, the term “intratumoral administration” refers to a mode of administration, in which the drug is administered directly into tumor tissue. The term “intratumoral administration” may in certain embodiments also refer to administration pre- or post-resection into or onto the tumor bed. When tumor boundary is not well defined, it is also understood that intratumoral administration includes administration to tissue adjacent to the tumor cells (“peri-tumoral administration”). Exemplary tumors for intratumoral administration are solid tumors and lymphomas, which are disclosed in more detail elsewhere herein. Administration may occur via injection.

As used herein the term “baseline tissue” refers to a tissue sample taken from, or adjacent to, the area to be treated prior to treatment. For example, a biopsy of tissue to be treated can be taken immediately prior to treatment. It is understood that it may not always be possible to take a reference sample from the respective area prior to treatment, so the term “baseline tissue” may also refer to a non-treated control tissue that may be taken from a comparable location from the same animal or may be taken from a comparable location of a different animal of the same species. It is understood that the term “animal” also covers human and in certain embodiments means mouse, rat, non-human primate or human.

As used herein the term “anti-tumor activity” means the ability to inhibit a tumor from growing larger, i.e. tumor growth inhibition or tumor stasis, or the ability to cause a reduction in the size of a tumor, i.e. tumor regression.

Any reference to a biologic drug herein, i.e., to a drug manufactured in, extracted from, or semisynthesized from biological sources such as a protein drug, also covers biosimilar versions of said drug.

For a drug containing a TLR7/8 agonist and other components, such as the TLR7/8 reversibly conjugated to a polymeric moiety, dose can be given with respect to mass or moles of the TLR7/8 agonist moiety or to the entire drug, e.g., to the TLR7/8 agonist conjugate. Unless otherwise apparent from the context, dosages for TLR7/8 agonist conjugates are given with respect to the TLR7/8 agonist moiety. Thus, the phrases “0.5 mg of TLR7/8 agonist” and “0.5 mg of TLR7/8 agonist conjugate” are used synonymously to give a dose of the TLR7/8 agonist conjugate based on the amount of TLR7/8 agonist moieties, which is 0.5 mg.

It is understood that the conjugates of the present invention are prodrugs.

As used herein the term “prodrug” refers to a drug moiety reversibly and covalently connected to a specialized protective group through a reversible prodrug linker moiety which is a linker moiety comprising a reversible linkage with the drug moiety and wherein the specialized protective group alters or eliminates undesirable properties in the parent molecule. This also includes the enhancement of desirable properties in the drug and the suppression of undesirable properties. The specialized non-toxic protective group may also be referred to as “carrier”, such as for example Z. A prodrug releases the reversibly and covalently bound drug moiety in the form of its corresponding drug. In other words, a prodrug is a conjugate comprising a drug moiety, which is covalently and reversibly conjugated to a carrier moiety via a reversible linker moiety, such as for example -L¹-, which covalent and reversible conjugation of the carrier to the reversible linker moiety is either directly or through a spacer, such as for example -L²-. The reversible linker may also be referred to as “reversible prodrug linker”. Such conjugate may release the formerly conjugated drug moiety in the form of a free drug, in which case the reversible linker or reversible prodrug linker is a traceless linker.

As used herein, the term “free form” of a drug means the drug in its unmodified, pharmacologically active form.

As used herein the term “spacer” refers to a moiety that connects at least two other moieties with each other.

As used herein, the terms “reversible”, “reversibly”, “degradable” or “degradably” with regard to the attachment of a first moiety to a second moiety means that the linkage that connects said first and second moiety is cleavable under physiological conditions, which physiological conditions are aqueous buffer at pH 7.4 and 37° C., with a half-life ranging from one day to three months, such as from two days to two months, such as from three days to one month. Such cleavage is in certain embodiments non-enzymatically. Accordingly, the term “stable” with regard to the attachment of a first moiety to a second moiety means that the linkage that connects said first and second moiety exhibits a half-life of more than three months under physiological conditions.

As used herein, the term “reagent” means a chemical compound, which comprises at least one functional group for reaction with the functional group of another chemical compound or drug. It is understood that a drug comprising a functional group is also a reagent.

As used herein, the term “moiety” means a part of a molecule, which lacks one or more atom(s) compared to the corresponding reagent. If, for example, a reagent of the formula “H—X—H” reacts with another reagent and becomes part of the reaction product, the corresponding moiety of the reaction product has the structure “H—X—” or “—X—”, whereas each “-” indicates attachment to another moiety. Accordingly, a drug moiety, such as TLR7/8 agonist moiety, is released from a reversible linkage as a drug, such as a TLR7/8 agonist drug.

It is understood that if the chemical structure of a group of atoms is provided and if this group of atoms is attached to two moieties or is interrupting a moiety, said chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety

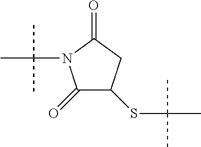

can be attached to two moieties or can interrupt a moiety either as

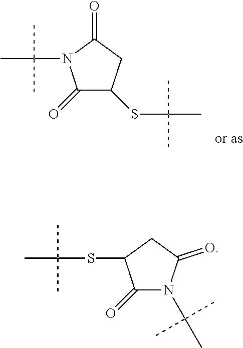

The term “substituted” as used herein means that one or more-H atom(s) of a molecule or moiety are replaced by a different atom or a group of atoms, which are referred to as “substituent”.

As used herein, the term “substituent” in certain embodiments refers to a moiety selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3})—, —S—, —N(R^{x3})—, —OC(OR³)(R^{x3})—, —N(R^{x3})C(O)N(R^{x3})—, and —OC(O)N(R^{x3})—;

—R^{x1}, —R^{x1a}, —R^{x1b} are independently of each other selected from the group consisting of —H, -TO, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each TO is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments a maximum of 6-H atoms of an optionally substituted molecule are independently replaced by a substituent, e.g. 5-H atoms are independently replaced by a substituent, 4-H atoms are independently replaced by a substituent, 3-H atoms are independently replaced by a substituent, 2-H atoms are independently replaced by a substituent, or 1-H atom is replaced by a substituent.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity. In certain embodiments the hydrogel is insoluble due to the presence of covalent chemical crosslinks.

As used herein the term “crosslinker” refers to a moiety that is a connection between different elements of a hydrogel, such as between two or more backbone moieties or between two or more hyaluronic acid strands.

As used herein the term “about” in combination with a numerical value is used to indicate a range ranging from and including the numerical value plus and minus no more than 25% of said numerical value, such as no more than plus and minus 20% of said numerical value or such as no more than plus and minus 10% of said numerical value. For example, the phrase “about 200” is used to mean a range ranging from and including 200+/−25%, i.e., ranging from and including 150 to 250; such as 200+/−20%, i.e., ranging from and including 160 to 240; such as ranging from and including 200+/−10%, i.e., ranging from and including 180 to 220. It is understood that a percentage given as “about 50%” does not mean “50%+/−25%”, i.e., ranging from and including 25 to 75%, but “about 50%” means ranging from and including 37.5 to 62.5%, i.e., plus and minus 25% of the numerical value which is 50. In case the range covered by “about X” is defined by one or two fractions at one or both ends of the range and it is clear that only integers are suitable values, the one or two fractions are rounded using the “round half up” rule to obtain the nearest integer.

The term “approx.” is used synonymously with “about”.

As used herein, the term “polymer” means a molecule comprising repeating structural units, i.e., the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes, for example, alternating copolymers in which monomers of different types alternate, periodic copolymers, in which monomers of different types are arranged in a repeating sequence; statistical copolymers, in which monomers of different types are arranged randomly; block copolymers, in which blocks of different homopolymers consisting of only one type of monomers are linked by a covalent bond; and gradient copolymers, in which the composition of different monomers changes gradually along a polymer chain. It is understood that a polymer may also comprise one or more other moieties, such as, for example, one or more functional groups. The term “polymer” also relates to a peptide or protein, even though the side chains of individual amino acid residues may be different. It is understood that for covalently crosslinked polymers, such as hydrogels, no meaningful molecular weight ranges can be provided.

As used herein, the term “polymeric” refers to a reagent or a moiety comprising one or more polymers or polymer moieties. A polymeric reagent or moiety may optionally also comprise one or more other moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

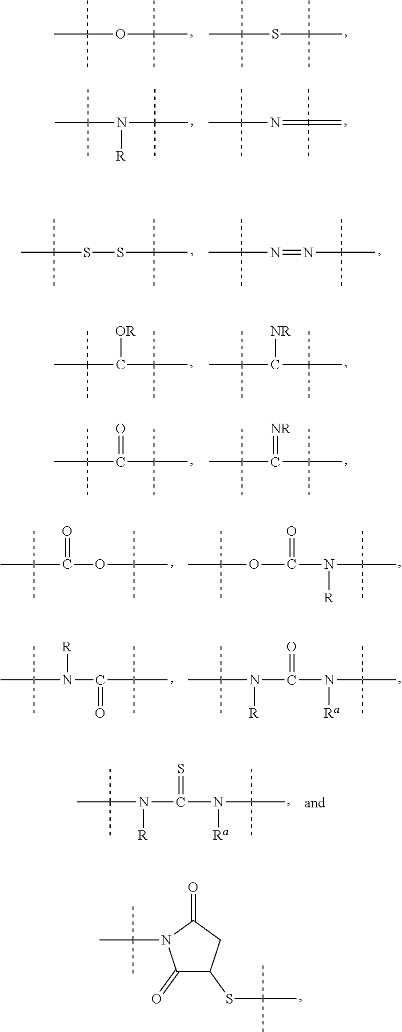

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

In certain embodiments a polymeric reagent or moiety may optionally also comprise one or more other moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

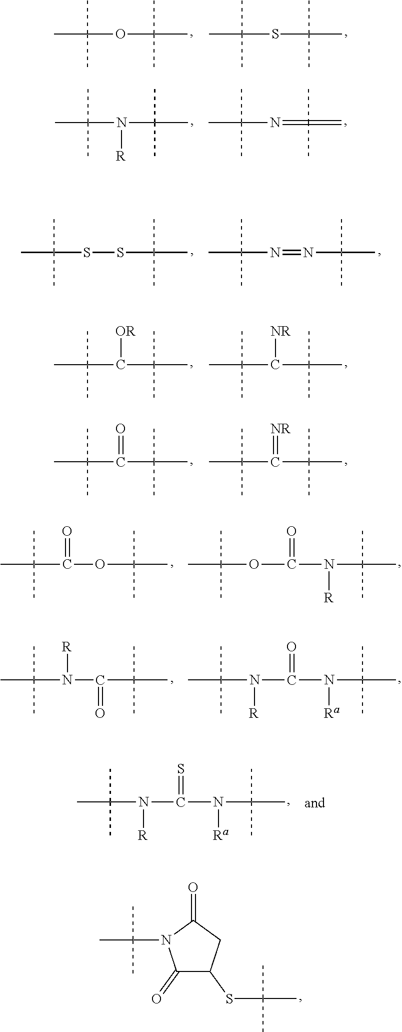

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lie. An integer for “x” given as “about x” means that the arithmetic mean numbers of monomers lie in a range of integers of x+/−25%, such as x+/−20% or such as x+/−10%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. Such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, or such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group consisting of

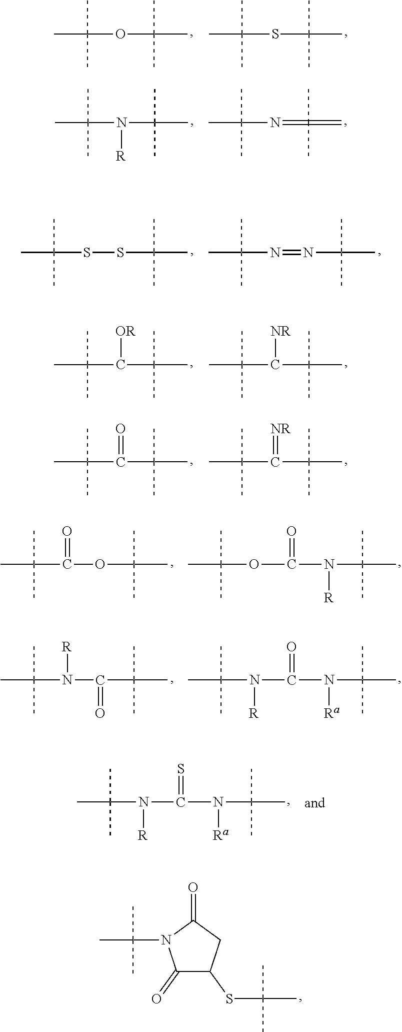

wherein
dashed lines indicate attachment to the remainder of the moiety or reagent, and —R and —R^{a} are independently of each other selected from the group consisting of —H, and C₁₋₆ alkyl; and
which moieties and linkages are optionally further substituted.

The terms “poly(alkylene glycol)-based”, “poly(propylene glycol)-based” and “hyaluronic acid-based” are used accordingly.

The term “interrupted” means that a moiety is inserted between two carbon atoms or—if the insertion is at one of the moiety's ends—between a carbon or heteroatom and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂-50 alkenyl group may optionally be replaced by a substituent as defined above. Optionally, a C₂₋₁₀ alkenyl, C₂₋₂₀ alkenyl or C₂-50 alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C═CH, —CH₂—C═CH, CH₂—CH₂—C≡CH and CH₂—C≡C—CH₃. When two moieties of a molecule are linked by the alkynyl group, then an example is-C═C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂-50 alkynyl group may optionally be replaced by a substituent as defined above. Optionally, one or more double bond(s) may occur. Optionally, a C₂₋₁₀ alkynyl, C₂₋₂₀ alkynyl or C₂-50 alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂-50 alkynyl may optionally be interrupted by one or more moieties which may be selected from the group consisting of

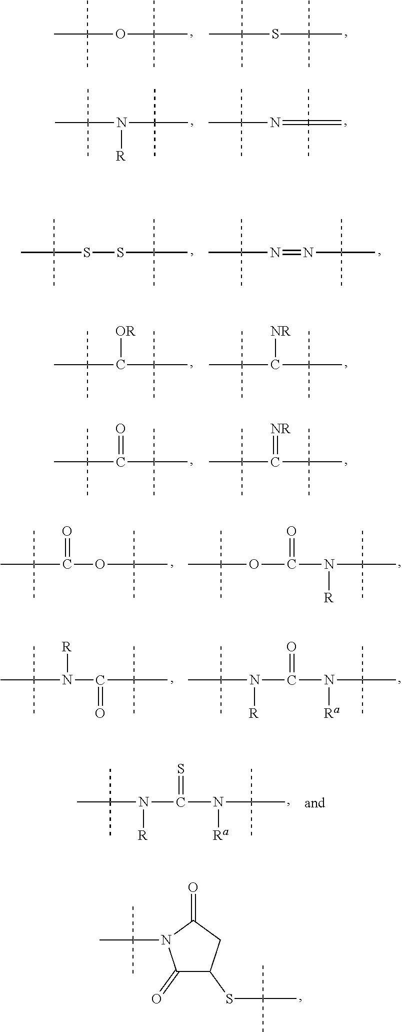

wherein
dashed lines indicate attachment to the remainder of the moiety or reagent; and —R and —R^{a} are independently of each other selected from the group consisting of —H and C₁₋₆ alkyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbornane or norbornene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In one embodiment an 8- to 30-membered carbopolycyclyl means a cyclic moiety of two, three, four or five rings. In another embodiment an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including-S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent.

Similarly, the term “8- to 30-membered heteropolycyclyl” or “8- to 30-membered heteropolycycle” means a heterocyclic moiety of more than two rings with 8 to 30 ring atoms, such as of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

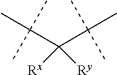

means that R^{x} and R^{y} form the following structure:

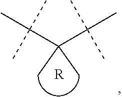

wherein R is C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

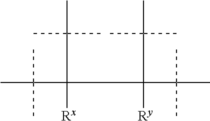

means that R^{x} and R^{y} form the following structure:

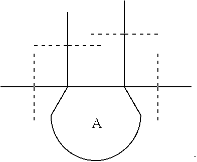

As used herein, “halogen” means fluoro, chloro, bromo or iodo. In certain embodiments halogen is fluoro or chloro.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the conjugates of the present invention comprise one or more acidic or basic groups, the invention also comprises their corresponding pharmaceutically or toxicologically acceptable salts, in particular their pharmaceutically utilizable salts. Thus, the conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine, amino acids, and quaternary ammonium salts, like tetrabutylammonium or cetyl trimethylammonium. Conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid, and other acids known to the person skilled in the art. For the person skilled in the art further methods are known for converting the basic group into a cation like the alkylation of an amine group resulting in a positively-charge ammonium group and an appropriate counterion of the salt. If the conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the conjugates of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and in certain embodiments means approved by a regulatory agency, such as the EMA (Europe) and/or the FDA (US) and/or any other national regulatory agency for use in animals, such as for use in humans.

As used herein, the term “excipient” refers to a diluent, adjuvant, or vehicle with which the therapeutic, such as a drug or prodrug, is administered. Such pharmaceutical excipient may be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino) ethanesulfonic acid), or may contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

The term “peptide” or “polypeptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

In general, the terms “comprise” or “comprising” also encompasses “consist of” or “consisting of”.

In certain embodiments the TLR7/8 agonist of the first, second and third embodiment is selected from the group consisting of CL075, CL097, poly(dT), resiquimod (R-848, VML600, S28463), MEDI9197 (3M-052), NKTR262, DV1001, IMO4200, IPH3201 and VTX1463.

In certain embodiments the TLR7/8 agonist of the first, second and third embodiment is resiquimod. Resiquimod has the following structure:

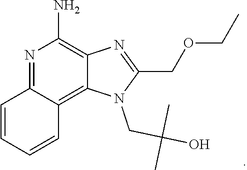 [SMILES: CCOCc1nc2c(N)nc3ccccc3c2n1CC(C)(C)O]

In certain embodiments a unit dosage form of the first aspect comprises a therapeutically effective amount of a TLR7/8 agonist conjugate or a pharmaceutically acceptable salt thereof and one or more excipients. In such case the unit dosage form comprises a pharmaceutical formulation comprising a therapeutically effective amount of a TLR7/8 agonist conjugate or a pharmaceutically acceptable salt thereof.

A unit dosage form of the first aspect comprises in certain embodiments 0.5 mg TLR7/8 agonist. In certain embodiments a unit dosage form of the first aspect comprises 1 mg TLR7/8 agonist. In certain embodiments a unit dosage form of the first aspect comprises 2 mg TLR7/8 agonist. A unit dosage form of the first aspect comprises in certain embodiments 0.5 mg TLR7/8 agonist and one or more excipients. In certain embodiments a unit dosage form of the first aspect comprises 1 mg TLR7/8 and one or more excipients. In certain embodiments a unit dosage form of the first aspect comprises 2 mg TLR7/8 and one or more excipients. It is understood that if the unit dosage form comprises for example 0.5 mg TLR7/8 agonist, such dose of 0.5 mg TLR7/8 is a unit dose.

In certain embodiments a unit dosage form of the first aspect comprises 0.5 mg resiquimod. In certain embodiments a unit dosage form of the first aspect comprises 1 mg resiquimod. In certain embodiments a unit dosage form of the first aspect comprises 2 mg resiquimod. In certain embodiments a unit dosage form of the first aspect comprises 0.5 mg resiquimod and one or more excipients. In certain embodiments a unit dosage form of the first aspect comprises 2 mg resiquimod and one or more excipients.

In certain embodiments the unit dosage form of the first aspect comprises a pharmaceutical formulation comprising the TLR7/8 agonist and the pharmaceutical formulation is a dry formulation, such as a lyophilized formulation. Such dry formulation is resuspended prior to administration to a patient using a liquid such as sterile water or a sterile buffer. Such resuspended formulation is in certain embodiments a suspension.

In certain embodiments the unit dosage form of the first aspect comprises a pharmaceutical formulation comprising the TLR7/8 agonist and the pharmaceutical formulation is a liquid formulation, such as a suspension.

In certain embodiments the unit dosage form of the first aspect comprises the TLR7/8 agonist conjugate in a volume ranging from 0.1 ml to 2 ml. In certain embodiments the unit dosage form of the first aspect comprises the TLR7/8 agonist conjugate in a volume ranging from 0.2 ml to 1.5 ml. In certain embodiments the unit dosage form of the first aspect comprises the TLR7/8 agonist conjugate in a volume of 0.25 ml. In certain embodiments the unit dosage form of the first aspect comprises the TLR7/8 agonist conjugate in a volume of 0.5 ml. In certain embodiments the unit dosage form of the first aspect comprises the TLR7/8 agonist conjugate in a volume of 1 ml.

In certain embodiments the unit dosage form of the first aspect comprises the TLR7/8 agonist conjugate in a volume ranging from 0.1 ml to 2 ml and the TLR7/8 agonist is resiquimod. In certain embodiments the unit dosage form of the first aspect comprises the TLR7/8 agonist conjugate in a volume ranging from 0.2 ml to 1.5 ml and the TLR7/8 agonist is resiquimod. In certain embodiments the unit dosage form of the first aspect comprises the TLR7/8 agonist conjugate in a volume of 0.25 ml and the TLR7/8 agonist is resiquimod. In certain embodiments the unit dosage form of the first aspect comprises the TLR7/8 agonist conjugate in a volume of 0.5 ml and the TLR7/8 agonist is resiquimod. In certain embodiments the unit dosage form of the first aspect comprises the TLR7/8 agonist conjugate in a volume of 1 ml and the TLR 7/8 agonist is resiquimod.

In certain embodiments a unit dosage form of the first aspect comprises 0.5 mg of the TLR7/8 agonist in a volume of 1 ml. In certain embodiments a unit dosage form of the first aspect comprises 0.5 mg of the TLR7/8 agonist in a volume of 0.25 ml. In certain embodiments a unit dosage form of the first aspect comprises 1 mg of the TLR7/8 agonist in a volume of 2 ml. In certain embodiments a unit dosage form of the first aspect comprises 1 mg of the TLR7/8 agonist in a volume of 0.5 ml.

In certain embodiments a unit dosage form of the first aspect comprises 0.5 mg of the TLR7/8 agonist, which TLR7/8 agonist is resiquimod, in a volume of 1 ml. In certain embodiments a unit dosage form of the first aspect comprises 0.5 mg of the TLR7/8 agonist, which TLR7/8 agonist is resiquimod, in a volume of 0.25 ml. In certain embodiments a unit dosage form of the first aspect comprises 1 mg of the TLR7/8 agonist, which TLR7/8 agonist is resiquimod, in a volume of 2 ml. In certain embodiments a unit dosage form of the first aspect comprises 1 mg of the TLR7/8 agonist, which TLR7/8 agonist is resiquimod, in a volume of 0.5 ml.

In certain embodiments the unit dosage form is a vial, a dual-chamber cartridge, an ampoule or a syringe comprising the unit dose.

In certain embodiments the unit dosage form is vial comprising the unit dose. Such vial may be a vial with a stopper, such as a bromo-butyl stopper, and a seal cap, such as an aluminium seal cap. In certain embodiments the vial is a glass vial, such as a type 1 glass vial. Such a vial may have a size of 1 ml, 2 ml or 5 ml, for example.

In certain embodiments the unit dosage form is stored at a temperature ranging from −80° C. to +25° C. In certain embodiments the unit dosage form is stored at a temperature ranging from −80° C. to 10° C. In certain embodiments the unit dosage form is stored at a temperature ranging from −80° C. to −60° C. In certain embodiments the unit dosage from is stored at a temperature ranging from −30° C. to −15° C. In certain embodiments the unit dosage form is stored at a temperature ranging from 0° C. to +10° C. In certain embodiments the unit dosage from is stored at about −80° C. In certain embodiments the unit dosage from is stored at −80° C. In certain embodiments the unit dosage form is stored at about −20° C. In certain embodiments the unit dosage form is stored at −20° C. In certain embodiments the unit dosage form is stored at a temperature ranging from 2° C. to 8° C.

In certain embodiments the unit dosage form of the first aspect is a vial comprising the TLR7/8 agonist conjugate or a pharmaceutically acceptable salt thereof. In certain embodiments the unit dosage form of the first aspect is a vial comprising 0.5 mg of the TLR7/8 agonist conjugate or a pharmaceutically acceptable salt thereof. In certain embodiments the unit dosage form of the first aspect is a vial comprising 1 mg of the TLR7/8 agonist conjugate or a pharmaceutically acceptable salt thereof. In certain embodiments the unit dosage form of the first aspect is a vial comprising 2 mg of the TLR7/8 agonist conjugate or a pharmaceutically acceptable salt thereof.

In certain embodiments the unit dosage form of the first aspect is a vial comprising the TLR7/8 agonist conjugate or a pharmaceutically acceptable salt thereof and one or more excipients. In certain embodiments the unit dosage form of the first aspect is a vial comprising 0.5 mg of the TLR7/8 agonist conjugate or a pharmaceutically acceptable salt thereof and one or more excipients. In certain embodiments the unit dosage form of the first aspect is a vial comprising 1 mg of the TLR7/8 agonist conjugate or a pharmaceutically acceptable salt thereof and one or more excipients. In certain embodiments the unit dosage form of the first aspect is a vial comprising 2 mg of the TLR7/8 agonist conjugate or a pharmaceutically acceptable salt thereof and one or more excipients.

In certain embodiments the unit dosage form of the first aspects is a vial comprising TLR7/8 agonist conjugate or a pharmaceutically acceptable salt thereof and the content of the vial is lyophilized. In certain embodiments the unit dosage form of the first aspects is a vial comprising 0.5 mg of the TLR7/8 agonist conjugate or a pharmaceutically acceptable salt thereof and the content of the vial is lyophilized. In certain embodiments the unit dosage form of the first aspects is a vial comprising 1 mg of the TLR7/8 agonist conjugate or a pharmaceutically acceptable salt thereof and the content of the vial is lyophilized. In certain embodiments the unit dosage form of the first aspects is a vial comprising 2 mg of the TLR7/8 agonist conjugate or a pharmaceutically acceptable salt thereof and the content of the vial is lyophilized.

In certain embodiments the unit dosage form of the first aspect is a vial comprising TLR7/8 agonist conjugate or a pharmaceutically acceptable salt thereof and one or more excipients and the content of the vial is lyophilized. In certain embodiments the unit dosage form of the first aspect is a vial comprising 0.5 mg of the TLR7/8 agonist conjugate or a pharmaceutically acceptable salt thereof and one or more excipients and the content of the vial is lyophilized. In certain embodiments the unit dosage form of the first aspect is a vial comprising 1 mg of the TLR7/8 agonist conjugate or a pharmaceutically acceptable salt thereof and one or more excipients and the content of the vial is lyophilized. In certain embodiments the unit dosage form of the first aspect is a vial comprising 2 mg of the TLR7/8 agonist conjugate or a pharmaceutically acceptable salt thereof and one or more excipients and the content of the vial is lyophilized.

In certain embodiments the unit dosage form of the first aspects is a vial comprising TLR7/8 agonist conjugate or a pharmaceutically acceptable salt thereof and the content of the vial is a liquid, such as a suspension. In certain embodiments the unit dosage form of the first aspects is a vial comprising 0.5 mg of the TLR7/8 agonist conjugate or a pharmaceutically acceptable salt thereof and the content of the vial is a liquid, such as a suspension. In certain embodiments the unit dosage form of the first aspects is a vial comprising 1 mg of the TLR7/8 agonist conjugate or a pharmaceutically acceptable salt thereof and the content of the vial is a liquid, such as a suspension. In certain embodiments the unit dosage form of the first aspects is a vial comprising 2 mg of the TLR7/8 agonist conjugate or a pharmaceutically acceptable salt thereof and the content of the vial is a liquid, such as a suspension.

In certain embodiments the unit dosage form of the first aspect is a vial comprising TLR7/8 agonist conjugate or a pharmaceutically acceptable salt thereof and one or more excipients and the content of the vial is a liquid, such as a suspension. In certain embodiments the unit dosage form of the first aspect is a vial comprising 0.5 mg of the TLR7/8 agonist conjugate or a pharmaceutically acceptable salt thereof and one or more excipients and the content of the vial is a liquid, such as a suspension. In certain embodiments the unit dosage form of the first aspect is a vial comprising 1 mg of the TLR7/8 agonist conjugate or a pharmaceutically acceptable salt thereof and one or more excipients and the content of the vial is a liquid, such as a suspension. In certain embodiments the unit dosage form of the first aspect is a vial comprising 2 mg of the TLR7/8 agonist conjugate or a pharmaceutically acceptable salt thereof and one or more excipients and the content of the vial is a liquid, such as a suspension.

In certain embodiments the unit dosage form of the first aspect is a vial comprising the TLR 7/8 agonist conjugate or a pharmaceutically acceptable salt thereof, wherein the TLR7/8 agonist is resiquimod. In certain embodiments the unit dosage form of the first aspect is a vial comprising 0.5 mg of the TLR7/8 agonist conjugate or a pharmaceutically acceptable salt thereof, wherein the TLR7/8 agonist is resiquimod. In certain embodiments the unit dosage form of the first aspect is a vial comprising 1 mg of the TLR7/8 agonist conjugate or a pharmaceutically acceptable salt thereof, wherein the TLR7/8 agonist is resiquimod. In certain embodiments the unit dosage form of the first aspect is a vial comprising 2 mg of the TLR7/8 agonist conjugate or a pharmaceutically acceptable salt thereof, wherein the TLR7/8 agonist is resiquimod.

In certain embodiments the unit dosage form of the first aspect is a vial comprising the TLR 7/8 agonist conjugate or a pharmaceutically acceptable salt thereof and one or more excipients, wherein the TLR7/8 agonist is resiquimod. In certain embodiments the unit dosage form of the first aspect is a vial comprising 0.5 mg of the TLR7/8 agonist conjugate or a pharmaceutically acceptable salt thereof and one or more excipients, wherein the TLR7/8 agonist is resiquimod. In certain embodiments the unit dosage form of the first aspect is a vial comprising 1 mg of the TLR7/8 agonist conjugate or a pharmaceutically acceptable salt thereof and one or more excipients, wherein the TLR7/8 agonist is resiquimod. In certain embodiments the unit dosage form of the first aspect is a vial comprising 2 mg of the TLR7/8 agonist conjugate or a pharmaceutically acceptable salt thereof and one or more excipients, wherein the TLR7/8 agonist is resiquimod.

In certain embodiments the unit dosage form of the first aspect is a vial comprising the TLR7/8 agonist conjugate or a pharmaceutically acceptable salt thereof, wherein the TLR7/8 agonist is resiquimod and the content of the vial is lyophilized. In certain embodiments the unit dosage form of the first aspect is a vial comprising 0.5 mg of the TLR7/8 agonist conjugate or a pharmaceutically acceptable salt thereof, wherein the TLR7/8 agonist is resiquimod and the content of the vial is lyophilized. In certain embodiments the unit dosage form of the first aspect is a vial comprising 1 mg of the TLR7/8 agonist conjugate or a pharmaceutically acceptable salt thereof, wherein the TLR7/8 agonist is resiquimod and the content of the vial is lyophilized. In certain embodiments the unit dosage form of the first aspect is a vial comprising 2 mg of the TLR7/8 agonist conjugate or a pharmaceutically acceptable salt thereof, wherein the TLR7/8 agonist is resiquimod and the content of the vial is lyophilized.

In certain embodiments the unit dosage form of the first aspect is a vial comprising the TLR7/8 agonist conjugate or a pharmaceutically acceptable salt thereof and one or more excipients, wherein the TLR7/8 agonist is resiquimod and the content of vial is lyophilized. In certain embodiments the unit dosage form of the first aspect is a vial comprising 0.5 mg of the TLR7/8 agonist conjugate or a pharmaceutically acceptable salt thereof and one or more excipients, wherein the TLR7/8 agonist is resiquimod and the content of vial is lyophilized. In certain embodiments the unit dosage form of the first aspect is a vial comprising 1 mg of the TLR7/8 agonist conjugate or a pharmaceutically acceptable salt thereof and one or more excipients, wherein the TLR7/8 agonist is resiquimod and the content of vial is lyophilized. In certain embodiments the unit dosage form of the first aspect is a vial comprising 2 mg of the TLR7/8 agonist conjugate or a pharmaceutically acceptable salt thereof and one or more excipients, wherein the TLR7/8 agonist is resiquimod and the content of vial is lyophilized.

In certain embodiments the unit dosage form of the first aspect is a vial comprising the TLR7/8 agonist conjugate or a pharmaceutically acceptable salt thereof, wherein the TLR7/8 agonist is resiquimod and the content of the vial is a liquid, such as a suspension. In certain embodiments the unit dosage form of the first aspect is a vial comprising 0.5 mg of the TLR7/8 agonist conjugate or a pharmaceutically acceptable salt thereof, wherein the TLR7/8 agonist is resiquimod and the content of the vial is a liquid, such as a suspension. In certain embodiments the unit dosage form of the first aspect is a vial comprising 1 mg of the TLR7/8 agonist conjugate or a pharmaceutically acceptable salt thereof, wherein the TLR7/8 agonist is resiquimod and the content of the vial is a liquid, such as a suspension. In certain embodiments the unit dosage form of the first aspect is a vial comprising 2 mg of the TLR7/8 agonist conjugate or a pharmaceutically acceptable salt thereof, wherein the TLR7/8 agonist is resiquimod and the content of the vial is a liquid, such as a suspension.

In certain embodiments the unit dosage form of the first aspect is a vial comprising the TLR7/8 agonist conjugate or a pharmaceutically acceptable salt thereof and one or more excipients, wherein the TLR7/8 agonist is resiquimod and the content of vial is a liquid, such as a suspension. In certain embodiments the unit dosage form of the first aspect is a vial comprising 0.5 mg of the TLR7/8 agonist conjugate or a pharmaceutically acceptable salt thereof and one or more excipients, wherein the TLR7/8 agonist is resiquimod and the content of vial is a liquid, such as a suspension. In certain embodiments the unit dosage form of the first aspect is a vial comprising 1 mg of the TLR7/8 agonist conjugate or a pharmaceutically acceptable salt thereof and one or more excipients, wherein the TLR7/8 agonist is resiquimod and the content of vial is a liquid, such as a suspension. In certain embodiments the unit dosage form of the first aspect is a vial comprising 2 mg of the TLR7/8 agonist conjugate or a pharmaceutically acceptable salt thereof and one or more excipients, wherein the TLR7/8 agonist is resiquimod and the content of vial is a liquid, such as a suspension.

In certain embodiments the unit dosage form of the first aspect is a dual-chamber cartridge comprising the TLR7/8 agonist conjugate or a pharmaceutically acceptable salt thereof in a first chamber. In certain embodiments the unit dosage form of the first aspect is a dual-chamber cartridge comprising 0.5 mg of the TLR7/8 agonist conjugate or a pharmaceutically acceptable salt thereof in a first chamber. In certain embodiments the unit dosage form of the first aspect is a dual-chamber cartridge comprising 1 mg of the TLR7/8 agonist conjugate or a pharmaceutically acceptable salt thereof in a first chamber. In certain embodiments the unit dosage form of the first aspect is a dual-chamber cartridge comprising 2 mg of the TLR7/8 agonist conjugate or a pharmaceutically acceptable salt thereof in a first chamber.

In certain embodiments the unit dosage form of the first aspect is a dual-chamber cartridge comprising the TLR7/8 agonist conjugate or a pharmaceutically acceptable salt thereof and one or more excipients in a first chamber. In certain embodiments the unit dosage form of the first aspect is a dual-chamber cartridge comprising 0.5 mg of the TLR7/8 agonist conjugate or a pharmaceutically acceptable salt thereof and one or more excipients in a first chamber. In certain embodiments the unit dosage form of the first aspect is a dual-chamber cartridge comprising 1 mg of the TLR7/8 agonist conjugate or a pharmaceutically acceptable salt thereof and one or more excipients in a first chamber. In certain embodiments the unit dosage form of the first aspect is a dual-chamber cartridge comprising 2 mg of the TLR7/8 agonist conjugate or a pharmaceutically acceptable salt thereof and one or more excipients in a first chamber.

In certain embodiments the unit dosage form of the first aspect is a dual-chamber cartridge comprising the TLR7/8 agonist conjugate or a pharmaceutically acceptable salt thereof in the first chamber and wherein the content of said first chamber is lyophilized. In certain embodiments the unit dosage form of the first aspect is a dual-chamber cartridge comprising 0.5 mg of the TLR7/8 agonist conjugate or a pharmaceutically acceptable salt thereof in the first chamber and wherein the content of said first chamber is lyophilized. In certain embodiments the unit dosage form of the first aspect is a dual-chamber cartridge comprising 1 mg of the TLR7/8 agonist conjugate or a pharmaceutically acceptable salt thereof in the first chamber and wherein the content of said first chamber is lyophilized. In certain embodiments the unit dosage form of the first aspect is a dual-chamber cartridge comprising 2 mg of the TLR7/8 agonist conjugate or a pharmaceutically acceptable salt thereof in the first chamber and wherein the content of said first chamber is lyophilized.

In certain embodiments the unit dosage form of the first aspect is a dual-chamber cartridge comprising the TLR7/8 agonist conjugate or a pharmaceutically acceptable salt thereof and one or more excipients in the first chamber and wherein the content of said first chamber is lyophilized. In certain embodiments the unit dosage form of the first aspect is a dual-chamber cartridge comprising 0.5 mg of the TLR7/8 agonist conjugate or a pharmaceutically acceptable salt thereof and one or more excipients in the first chamber and wherein the content of said first chamber is lyophilized. In certain embodiments the unit dosage form of the first aspect is a dual-chamber cartridge comprising 1 mg of the TLR7/8 agonist conjugate or a pharmaceutically acceptable salt thereof and one or more excipients in the first chamber and wherein the content of said first chamber is lyophilized. In certain embodiments the unit dosage form of the first aspect is a dual-chamber cartridge comprising 2 mg of the TLR7/8 agonist conjugate or a pharmaceutically acceptable salt thereof and one or more excipients in the first chamber and wherein the content of said first chamber is lyophilized.

In certain embodiments the unit dosage form of the first aspect is a dual-chamber cartridge comprising the TLR7/8 agonist conjugate or a pharmaceutically acceptable salt thereof in a first chamber, wherein the TLR7/8 agonist is resiquimod. In certain embodiments the unit dosage form of the first aspect is a dual-chamber cartridge comprising 0.5 mg of the TLR7/8 agonist conjugate or a pharmaceutically acceptable salt thereof in a first chamber, wherein the TLR7/8 agonist is resiquimod. In certain embodiments the unit dosage form of the first aspect is a dual-chamber cartridge comprising 1 mg of the TLR7/8 agonist conjugate or a pharmaceutically acceptable salt thereof in a first chamber, wherein the TLR7/8 agonist is resiquimod. In certain embodiments the unit dosage form of the first aspect is a dual-chamber cartridge comprising 2 mg of the TLR7/8 agonist conjugate or a pharmaceutically acceptable salt thereof in a first chamber, wherein the TLR7/8 agonist is resiquimod.

In certain embodiments the unit dosage form of the first aspect is a dual-chamber cartridge comprising the TLR7/8 agonist conjugate or a pharmaceutically acceptable salt thereof and one or more excipients in a first chamber, wherein the TLR7/8 agonist is resiquimod. In certain embodiments the unit dosage form of the first aspect is a dual-chamber cartridge comprising 0.5 mg of the TLR7/8 agonist conjugate or a pharmaceutically acceptable salt thereof and one or more excipients in a first chamber, wherein the TLR7/8 agonist is resiquimod. In certain embodiments the unit dosage form of the first aspect is a dual-chamber cartridge comprising 1 mg of the TLR7/8 agonist conjugate or a pharmaceutically acceptable salt thereof and one or more excipients in a first chamber, wherein the TLR7/8 agonist is resiquimod. In certain embodiments the unit dosage form of the first aspect is a dual-chamber cartridge comprising 2 mg of the TLR7/8 agonist conjugate or a pharmaceutically acceptable salt thereof and one or more excipients in a first chamber, wherein the TLR7/8 agonist is resiquimod.

In certain embodiments the unit dosage form of the first aspect is a dual-chamber cartridge comprising the TLR7/8 agonist conjugate or a pharmaceutically acceptable salt thereof in the first chamber, wherein the TLR7/8 agonist is resiquimod and wherein the content of said first chamber is lyophilized. In certain embodiments the unit dosage form of the first aspect is a dual-chamber cartridge comprising 0.5 mg of the TLR7/8 agonist conjugate or a pharmaceutically acceptable salt thereof in the first chamber, wherein the TLR7/8 agonist is resiquimod and wherein the content of said first chamber is lyophilized. In certain embodiments the unit dosage form of the first aspect is a dual-chamber cartridge comprising 1 mg of the TLR7/8 agonist conjugate or a pharmaceutically acceptable salt thereof in the first chamber, wherein the TLR7/8 agonist is resiquimod and wherein the content of said first chamber is lyophilized. In certain embodiments the unit dosage form of the first aspect is a dual-chamber cartridge comprising 2 mg of the TLR7/8 agonist conjugate or a pharmaceutically acceptable salt thereof in the first chamber, wherein the TLR7/8 agonist is resiquimod and wherein the content of said first chamber is lyophilized.

In certain embodiments the unit dosage form of the first aspect is a dual-chamber cartridge comprising the TLR7/8 agonist conjugate or a pharmaceutically acceptable salt thereof and one or more excipients in the first chamber, wherein the TLR7/8 agonist is resiquimod and wherein the content of said first chamber is lyophilized. In certain embodiments the unit dosage form of the first aspect is a dual-chamber cartridge comprising 0.5 mg of the TLR7/8 agonist conjugate or a pharmaceutically acceptable salt thereof and one or more excipients in the first chamber, wherein the TLR7/8 agonist is resiquimod and wherein the content of said first chamber is lyophilized. In certain embodiments the unit dosage form of the first aspect is a dual-chamber cartridge comprising 1 mg of the TLR7/8 agonist conjugate or a pharmaceutically acceptable salt thereof and one or more excipients in the first chamber, wherein the TLR7/8 agonist is resiquimod and wherein the content of said first chamber is lyophilized. In certain embodiments the unit dosage form of the first aspect is a dual-chamber cartridge comprising 2 mg of the TLR7/8 agonist conjugate or a pharmaceutically acceptable salt thereof and one or more excipients in the first chamber, wherein the TLR7/8 agonist is resiquimod and wherein the content of said first chamber is lyophilized.

In all embodiments the second chamber of a dual-chamber cartridge may comprise buffer or water for resuspension.

In certain embodiments the unit dosage form of the first aspect comprises a pharmaceutical formulation comprising the TLR7/8 agonist, such as resiquimod, and the pharmaceutical formulation is a liquid formulation or a resuspended formulation with a concentration ranging from 0.5 mg TLR7/8 agonist, such as resiquimod, per ml to 2 mg TLR7/8 agonist, such as resiquimod, per ml. In certain embodiments the concentration is 0.5 mg TLR7/8 agonist, such as resiquimod, per ml. In certain embodiments the concentration is 1 mg TLR7/8 agonist, such as resiquimod, per ml.

In certain embodiments the unit dosage form of the first aspect is for use in the treatment of cancer, in particular of a solid tumor. In certain embodiment the unit dose of the unit dosage form of the first aspect is administered via intratumoral injection, such as by using a fanning technique. In certain embodiments the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma. In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC). In certain embodiments the solid tumor is selected from the group consisting of pancreatic cancer, prostate cancer, melanoma, SCCHN, cutaneous squamous cell cancer (cSCC) and cervical cancer. In certain embodiments the solid tumor is a pancreatic cancer. In certain embodiments the solid tumor is prostate cancer. In certain embodiments the solid tumor is a melanoma. In certain embodiments the solid tumor is a SCCHN. In certain embodiments the solid tumor is a cSCC. In certain embodiments the solid tumor is cervical cancer.

In certain embodiments the unit dose of the unit dosage form of the first aspect is for use in the treatment of cancer, wherein the unit dose is administered to the patient every two to four weeks. In certain embodiments the unit dose of the unit dosage form of the first aspect is for use in the treatment of cancer, wherein the unit dose is administered to the patient every two weeks. In certain embodiments the unit dose of the unit dosage form of the first aspect is for use in the treatment of cancer, wherein the unit dose is administered to the patient every three weeks. In certain embodiments the unit dose of the unit dosage form of the first aspect is for use in the treatment of cancer, wherein the unit dose is administered to the patient every four weeks.

In certain embodiments the unit dose of the unit dosage form of the first aspect is for use in the treatment of cancer, wherein the unit dose is administered to the patient every five weeks. In certain embodiments the unit dose of the unit dosage form of the first aspect is for use in the treatment of cancer, wherein the unit dose is administered to the patient every six weeks. In certain embodiments the unit dose of the unit dosage form of the first aspect is for use in the treatment of cancer, wherein the unit dose is administered to the patient every seven weeks. In certain embodiments the unit dose of the unit dosage form of the first aspect is for use in the treatment of cancer, wherein the unit dose is administered to the patient every eight weeks. In certain embodiments the unit dose of the unit dosage form of the first aspect is for use in the treatment of cancer, wherein the unit dose is administered to the patient every nine weeks. In certain embodiments the unit dose of the unit dosage form of the first aspect is for use in the treatment of cancer, wherein the unit dose is administered to the patient every ten weeks. In certain embodiments the unit dose of the unit dosage form of the first aspect is for use in the treatment of cancer, wherein the unit dose is administered to the patient every twelve weeks.

In certain embodiments the unit dose of the unit dosage form of the first aspect is for use in the treatment of cancer, wherein the unit dose is administered to the patient every six months. In certain embodiments the unit dose of the unit dosage form of the first aspect is for use in the treatment of cancer, wherein the unit dose is administered to the patient every seven months. In certain embodiments the unit dose of the unit dosage form of the first aspect is for use in the treatment of cancer, wherein the unit dose is administered to the patient every eight months. In certain embodiments the unit dose of the unit dosage form of the first aspect is for use in the treatment of cancer, wherein the unit dose is administered to the patient every nine months. In certain embodiments the unit dose of the unit dosage form of the first aspect is for use in the treatment of cancer, wherein the unit dose is administered to the patient every ten months. In certain embodiments the unit dose of the unit dosage form of the first aspect is for use in the treatment of cancer, wherein the unit dose is administered to the patient every eleven months. In certain embodiments the unit dose of the unit dosage form of the first aspect is for use in the treatment of cancer, wherein the unit dose is administered to the patient once a year.

In certain embodiments the unit dose of the unit dosage form of the first aspect is for use in the treatment of cancer, wherein the unit dose is administered at a frequency dependent on disease progression.

In certain embodiments the unit dose is 0.5 mg TLR7/8 agonist per tumor. In certain embodiments the unit dose is 1 mg TLR7/8 agonist per tumor. Such unit dose may be administered via intratumoral injection, such as by using a fanning technique.

In certain embodiments the treatment of cancer comprises administration of the unit dose to one tumor of the patient. In certain embodiments the treatment of cancer comprises administration of the unit dose to more than one tumor of the patient, such as to two tumors, three tumors, four tumors or five tumors or to all tumors of a patient that have a sufficient size and are accessible for treatment. If the treatment comprises administration to more than one tumor per patient, such administrations may occur within at most four hours, such as within three hours, within two hours or within one hour. In certain embodiments administration to more than one tumor occurs consecutively without breaks between administrations. In certain embodiments administrations to different tumors of a patient occur at different times, such that for example the time between one administration and the following administration ranges from one day to three weeks and is in certain embodiments one week, two weeks or three weeks.

In certain embodiments the treatment comprises one administration of a unit dose to a tumor of the patient, such as a dose of 0.5 mg TLR7/8 agonist, such as resiquimod, per tumor, which unit dose is administered as one intratumoral injection. In certain embodiments the treatment comprises repeated administrations of a unit dose to a tumor of the patient, such as repeated administrations of 0.5 mg TLR7/8 agonist, such as resiquimod, per tumor administered via intratumoral injection, wherein each unit dose is administered as one intratumoral injection. Such treatment with repeated administrations per tumor means in certain embodiment two, three, four, five, six, seven, eight, nine, ten, eleven, twelve or more administrations to a particular tumor of the patient. In certain embodiments more than one tumor of the patient are treated with such repeated administrations.

In certain embodiments the unit dose of the unit dosage form of the first aspect comprises 0.5 mg of the TLR7/8 agonist conjugate and is for use in the treatment of cancer, wherein the unit dose is administered to the patient every two to four weeks. In certain embodiments the unit dose of the unit dosage form of the first aspect comprises 0.5 mg of the TLR7/8 agonist conjugate and is for use in the treatment of cancer, wherein the unit dose is administered to the patient every two weeks. In certain embodiments the unit dose of the unit dosage form of the first aspect comprises 0.5 mg of the TLR7/8 agonist conjugate and is for use in the treatment of cancer, wherein the unit dose is administered to the patient every three weeks. In certain embodiments the unit dose of the unit dosage form of the first aspect comprises 0.5 mg of the TLR7/8 agonist conjugate and is for use in the treatment of cancer, wherein the unit dose is administered to the patient every four weeks.

In certain embodiments the unit dose of the unit dosage form of the first aspect is for use in the treatment of cancer, wherein the unit dose is administered to the patient every two to four weeks via intratumoral administration. In certain embodiments the unit dose of the unit dosage form of the first aspect is for use in the treatment of cancer, wherein the unit dose is administered to the patient every two weeks via intratumoral administration. In certain embodiments the unit dose of the unit dosage form of the first aspect is for use in the treatment of cancer, wherein the unit dose is administered to the patient every three weeks via intratumoral administration. In certain embodiments the unit dose of the unit dosage form of the first aspect is for use in the treatment of cancer, wherein the unit dose is administered to the patient every four weeks via intratumoral administration.

In certain embodiments the unit dose of the unit dosage form of the first aspect is for use in the treatment of cancer, wherein the unit dose comprises 0.5 mg TLR7/8 agonist conjugate and is administered to the patient every two to four weeks via intratumoral administration. In certain embodiments the unit dose of the unit dosage form of the first aspect is for use in the treatment of cancer, wherein the unit dose comprises 0.5 mg TLR7/8 agonist conjugate and is administered to the patient every two weeks via intratumoral administration. In certain embodiments the unit dose of the unit dosage form of the first aspect is for use in the treatment of cancer, wherein the unit dose comprises 0.5 mg TLR7/8 agonist conjugate and is administered to the patient every three weeks via intratumoral administration. In certain embodiments the unit dose of the unit dosage form of the first aspect is for use in the treatment of cancer, wherein the unit dose comprises 0.5 mg TLR7/8 agonist conjugate and is administered to the patient every four weeks via intratumoral administration.

In certain embodiments the unit dose of the unit dosage form of the first aspect is for use in the treatment of cancer, wherein the unit dose is administered to the patient every two to four weeks via intratumoral administration using a fanning technique. In certain embodiments the unit dose of the unit dosage form of the first aspect is for use in the treatment of cancer, wherein the unit dose is administered to the patient every two weeks via intratumoral administration using a fanning technique. In certain embodiments the unit dose of the unit dosage form of the first aspect is for use in the treatment of cancer, wherein the unit dose is administered to the patient every three weeks via intratumoral administration using a fanning technique. In certain embodiments the unit dose of the unit dosage form of the first aspect is for use in the treatment of cancer, wherein the unit dose is administered to the patient every four weeks via intratumoral administration using a fanning technique.

In certain embodiments the unit dose of the unit dosage form of the first aspect is for use in the treatment of cancer, wherein the unit dose comprises 0.5 mg TLR7/8 agonist conjugate and is administered to the patient every two to four weeks via intratumoral administration using a fanning technique. In certain embodiments the unit dose of the unit dosage form of the first aspect is for use in the treatment of cancer, wherein the unit dose comprises 0.5 mg TLR7/8 agonist conjugate and is administered to the patient every two weeks via intratumoral administration using a fanning technique. In certain embodiments the unit dose of the unit dosage form of the first aspect is for use in the treatment of cancer, wherein the unit dose comprises 0.5 mg TLR7/8 agonist conjugate and is administered to the patient every three weeks via intratumoral administration using a fanning technique. In certain embodiments the unit dose of the unit dosage form of the first aspect is for use in the treatment of cancer, wherein the unit dose comprises 0.5 mg TLR7/8 agonist conjugate and is administered to the patient every four weeks via intratumoral administration using a fanning technique.

In certain embodiments the unit dose of the unit dosage form of the first aspect is for use in the treatment of cancer, wherein the unit dose is administered to the patient every two to four weeks and wherein the treatment is a cotreatment with an immune checkpoint inhibitor, which may be given prior to, together with or after administration of the TLR7/8 agonist conjugate. In certain embodiments the unit dose of the unit dosage form of the first aspect is for use in the treatment of cancer, wherein the unit dose is administered to the patient every two weeks and wherein the treatment is a cotreatment with an immune checkpoint inhibitor, which may be given prior to, together with or after administration of the TLR7/8 agonist conjugate. In certain embodiments the unit dose of the unit dosage form of the first aspect is for use in the treatment of cancer, wherein the unit dose is administered to the patient every three weeks and wherein the treatment is a cotreatment with an immune checkpoint inhibitor, which may be given prior to, together with or after administration of the TLR7/8 agonist conjugate. In certain embodiments the unit dose of the unit dosage form of the first aspect is for use in the treatment of cancer, wherein the unit dose is administered to the patient every four weeks and wherein the treatment is a cotreatment with an immune checkpoint inhibitor, which may be given prior to, together with or after administration of the TLR7/8 agonist conjugate.

In certain embodiments the unit dose of the unit dosage form of the first aspect is for use in the treatment of cancer, wherein the unit dose comprises 0.5 mg TLR7/8 agonist conjugate and is administered to the patient every two to four weeks and wherein the treatment is a cotreatment with an immune checkpoint inhibitor, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every two weeks and wherein the treatment is a cotreatment with an immune checkpoint inhibitor, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every three weeks and wherein the treatment is a cotreatment with an immune checkpoint inhibitor, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every four weeks and wherein the treatment is a cotreatment with an immune checkpoint inhibitor, which may be given prior to, together with or after administration of the TLR7/8 agonist conjugate.

In certain embodiments the unit dose of the unit dosage form of the first aspect is for use in the treatment of cancer, wherein the unit dose is administered to the patient every two to four weeks and wherein the treatment is a cotreatment with an inhibitor of PD-1 or an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose is administered to the patient every two weeks and wherein the treatment is a cotreatment with an inhibitor of PD-1 or an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose is administered to the patient every three weeks and wherein the treatment is a cotreatment with an inhibitor of PD-1 or an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose is administered to the patient every four weeks and wherein the treatment is a cotreatment with an inhibitor of PD-1 or an inhibitor of PD-L1, which may be given prior to, together with or after administration of the TLR7/8 agonist conjugate.

In certain embodiments the unit dose of the unit dosage form of the first aspect is for use in the treatment of cancer, wherein the unit dose comprises 0.5 mg TLR7/8 agonist conjugate and is administered to the patient every two to four weeks and wherein the treatment is a cotreatment with an inhibitor of PD-1 or an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every two weeks and wherein the treatment is a cotreatment with an inhibitor of PD-1 or an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every three weeks and wherein the treatment is a cotreatment with an inhibitor of PD-1 or an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every four weeks and wherein the treatment is a cotreatment with an inhibitor of PD-1 or an inhibitor of PD-L1, which may be given prior to, together with or after administration of the TLR7/8 agonist conjugate.

An inhibitor of PD-1 (programmed cell death protein 1) may be selected from the group consisting of pembrolizumab, nivolumab, pidilizumab, AMP-224, BMS-936559, cemiplimab and PDR001. An inhibitor of PD-L1 (programmed cell death ligand 1) may be selected from the group consisting of MDX-1105, MEDI4736, atezolizumab, avelumab, BMS-936559 and durvalumab.

In certain embodiments the unit dose of the unit dosage form of the first aspect is for use in the treatment of cancer, wherein the unit dose is administered to the patient every two to four weeks and wherein the treatment is a cotreatment with an inhibitor of PD-1, which may be given prior to, together with or after administration of the TLR7/8 agonist conjugate. In certain embodiments the unit dose of the unit dosage form of the first aspect is for use in the treatment of cancer, wherein the unit dose is administered to the patient every two weeks and wherein the treatment is a cotreatment with an inhibitor of PD-1, which may be given prior to, together with or after administration of the TLR7/8 agonist conjugate. In certain embodiments the unit dose of the unit dosage form of the first aspect is for use in the treatment of cancer, wherein the unit dose is administered to the patient every three weeks and wherein the treatment is a cotreatment with an inhibitor of PD-1, which may be given prior to, together with or after administration of the TLR7/8 agonist conjugate. In certain embodiments the unit dose of the unit dosage form of the first aspect is for use in the treatment of cancer, wherein the unit dose is administered to the patient every four weeks and wherein the treatment is a cotreatment with an inhibitor of PD-1, which may be given prior to, together with or after administration of the TLR7/8 agonist conjugate.

In certain embodiments the unit dose of the unit dosage form of the first aspect is for use in the treatment of cancer, wherein the unit dose comprises 0.5 mg TLR7/8 agonist conjugate and is administered to the patient every two to four weeks and wherein the treatment is a cotreatment with an inhibitor of PD-1, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every two weeks and wherein the treatment is a cotreatment with an inhibitor of PD-1, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every three weeks and wherein the treatment is a cotreatment with an inhibitor of PD-1, which may be given prior to, together with or after administration of the TLR7/8 agonist conjugate. In certain embodiments the unit dose of the unit dosage form of the first aspect is for use in the treatment of cancer, wherein the unit dose comprises 0.5 mg TLR7/8 agonist conjugate and is administered to the patient every four weeks and wherein the treatment is a cotreatment with an inhibitor of PD-1, which may be given prior to, together with or after administration of the TLR7/8 agonist conjugate.

In certain embodiments the unit dose of the unit dosage form of the first aspect is for use in the treatment of cancer, wherein the unit dose is administered to the patient every two to four weeks and wherein the treatment is a cotreatment with an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose is administered to the patient every two weeks and wherein the treatment is a cotreatment with an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose is administered to the patient every three weeks and wherein the treatment is a cotreatment with an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dose is administered to the patient every four weeks and wherein the treatment is a cotreatment with an inhibitor of PD-L1, which may be given prior to, together with or after administration of the TLR7/8 agonist conjugate.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the treatment is a cotreatment with an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the treatment is a cotreatment with an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the treatment is a cotreatment with an inhibitor of PD-L1, which may be given prior to, together with or after administration of the TLR7/8 agonist conjugate. In certain embodiments the unit dose of the unit dosage form of the first aspect is for use in the treatment of cancer, wherein the unit dosage comprises 0.5 mg TLR7/8 agonist conjugate and is administered to the patient every four weeks and wherein the treatment is a cotreatment with an inhibitor of PD-L1, which may be given prior to, together with or after administration of the TLR7/8 agonist conjugate.

In certain embodiments the unit dose of the unit dosage form of the first aspect is for use in the treatment of cancer, wherein the unit dose is administered to the patient every two to four weeks and wherein the treatment is a cotreatment with pembrolizumab, which may be given prior to, together with or after administration of the TLR7/8 agonist conjugate. In certain embodiments the unit dose of the unit dosage form of the first aspect is for use in the treatment of cancer, wherein the unit dose is administered to the patient every two weeks and wherein the treatment is a cotreatment with pembrolizumab, which may be given prior to, together with or after administration of the TLR7/8 agonist conjugate. In certain embodiments the unit dose of the unit dosage form of the first aspect is for use in the treatment of cancer, wherein the unit dose is administered to the patient every three weeks and wherein the treatment is a cotreatment with pembrolizumab, which may be given prior to, together with or after administration of the TLR7/8 agonist conjugate. In certain embodiments the unit dose of the unit dosage form of the first aspect is for use in the treatment of cancer, wherein the unit dose is administered to the patient every four weeks and wherein the treatment is a cotreatment with pembrolizumab, which may be given prior to, together with or after administration of the TLR7/8 agonist conjugate.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the treatment is a cotreatment with pembrolizumab, which may be given prior to, together with or after administration of the TLR7/8 agonist conjugate. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the treatment is a cotreatment with pembrolizumab, which may be given prior to, together with or after administration of the TLR7/8 agonist conjugate. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the treatment is a cotreatment with pembrolizumab, which may be given prior to, together with or after administration of the TLR7/8 agonist conjugate. In certain embodiments the unit dose of the unit dosage form of the first aspect is for use in the treatment of cancer, wherein the unit dosage comprises 0.5 mg TLR7/8 agonist conjugate and is administered to the patient every four weeks and wherein the treatment is a cotreatment with pembrolizumab, which may be given prior to, together with or after administration of the TLR7/8 agonist conjugate.

Pembrolizumab may be given as per the prescription information, such as 200 mg pembrolizumab per intravenous infusion, for example every three weeks. The infusion time may range from 15 minutes to 4 hours, such as from 30 minutes to one hour and is typically about 30 minutes. In general, pembrolizumab is administered with the dose, administration frequency and form of administration approved for a given indication.

In certain embodiments the unit dose of the unit dosage form of the first aspect is for use in the treatment of cancer, wherein the unit dose is administered to the patient every two to four weeks and wherein the TLR7/8 agonist is resiquimod. In certain embodiments the unit dose of the unit dosage form of the first aspect is for use in the treatment of cancer, wherein the unit dose is administered to the patient every two weeks and wherein the TLR7/8 agonist is resiquimod. In certain embodiments the unit dose of the unit dosage form of the first aspect is for use in the treatment of cancer, wherein the unit dose is administered to the patient every three weeks and wherein the TLR7/8 agonist is resiquimod. In certain embodiments the unit dose of the unit dosage form of the first aspect is for use in the treatment of cancer, wherein the unit dose is administered to the patient every four weeks and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the TLR7/8 agonist is resiquimod. In certain embodiments the unit dose of the unit dosage form of the first aspect is for use in the treatment of cancer, wherein the unit dose is administered to the patient every four weeks and wherein the TLR7/8 agonist is resiquimod.

In certain embodiments the unit dose of the unit dosage form of the first aspect is for use in the treatment of cancer, wherein the unit dose is administered to the patient every two to four weeks via intratumoral administration and wherein the TLR7/8 agonist is resiquimod. In certain embodiments the unit dose of the unit dosage form of the first aspect is for use in the treatment of cancer, wherein the unit dose is administered to the patient every two weeks via intratumoral administration and wherein the TLR7/8 agonist is resiquimod. In certain embodiments the unit dose of the unit dosage form of the first aspect is for use in the treatment of cancer, wherein the unit dose is administered to the patient every three weeks via intratumoral administration and wherein the TLR7/8 agonist is resiquimod. In certain embodiments the unit dose of the unit dosage form of the first aspect is for use in the treatment of cancer, wherein the unit dose is administered to the patient every four weeks via intratumoral administration and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks via intratumoral administration and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks via intratumoral administration and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks via intratumoral administration and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every four weeks via intratumoral administration and wherein the TLR7/8 agonist is resiquimod.

In certain embodiments the unit dose of the unit dosage form of the first aspect is for use in the treatment of cancer, wherein the unit dose is administered to the patient every two to four weeks via intratumoral administration using a fanning technique and wherein the TLR7/8 agonist is resiquimod. In certain embodiments the unit dose of the unit dosage form of the first aspect is for use in the treatment of cancer, wherein the unit dose is administered to the patient every two weeks via intratumoral administration using a fanning technique and wherein the TLR7/8 agonist is resiquimod. In certain embodiments the unit dose of the unit dosage form of the first aspect is for use in the treatment of cancer, wherein the unit dose is administered to the patient every three weeks via intratumoral administration using a fanning technique and wherein the TLR7/8 agonist is resiquimod. In certain embodiments the unit dose of the unit dosage form of the first aspect is for use in the treatment of cancer, wherein the unit dose is administered to the patient every four weeks via intratumoral administration using a fanning technique and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks via intratumoral administration using a fanning technique and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks via intratumoral administration using a fanning technique and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks via intratumoral administration using a fanning technique and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every four weeks via intratumoral administration using a fanning technique and wherein the TLR7/8 agonist is resiquimod.

In certain embodiments the unit dose of the unit dosage form of the first aspect is for use in the treatment of cancer, wherein the unit dose is administered to the patient every two to four weeks and wherein the treatment is a cotreatment with an immune checkpoint inhibitor, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every two weeks and wherein the treatment is a cotreatment with an immune checkpoint inhibitor, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every three weeks and wherein the treatment is a cotreatment with an immune checkpoint inhibitor, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every four weeks and wherein the treatment is a cotreatment with an immune checkpoint inhibitor, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the treatment is a cotreatment with an immune checkpoint inhibitor, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the treatment is a cotreatment with an immune checkpoint inhibitor, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the treatment is a cotreatment with an immune checkpoint inhibitor, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every four weeks and wherein the treatment is a cotreatment with an immune checkpoint inhibitor, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dose is administered to the patient every two to four weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every two weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every three weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every four weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every four weeks and wherein the treatment is a cotreatment with an inhibitor of PD-1 or an inhibitor of PD-L1, which may be given prior to, together with or after administration of the TLR7/8 agonist conjugate and wherein the TLR7/8 agonist is resiquimod.

Examples for the inhibitor of PD-1 and PD-L1 are as described elsewhere herein.

In certain embodiments the unit dose of the unit dosage form of the first aspect is for use in the treatment of cancer, wherein the unit dose is administered to the patient every two to four weeks and wherein the treatment is a cotreatment with an inhibitor of PD-1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every two weeks and wherein the treatment is a cotreatment with an inhibitor of PD-1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every three weeks and wherein the treatment is a cotreatment with an inhibitor of PD-1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every four weeks and wherein the treatment is a cotreatment with an inhibitor of PD-1, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the treatment is a cotreatment with an inhibitor of PD-1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the treatment is a cotreatment with an inhibitor of PD-1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the treatment is a cotreatment with an inhibitor of PD-1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every four weeks and wherein the treatment is a cotreatment with an inhibitor of PD-1, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dose is administered to the patient every two to four weeks and wherein the treatment is a cotreatment with an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every two weeks and wherein the treatment is a cotreatment with an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every three weeks and wherein the treatment is a cotreatment with an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every four weeks and wherein the treatment is a cotreatment with an inhibitor of PD-L1, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the treatment is a cotreatment with an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the treatment is a cotreatment with an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the treatment is a cotreatment with an inhibitor of PD-L1, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every four weeks and wherein the treatment is a cotreatment with an inhibitor of PD-L1, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dose is administered to the patient every two to four weeks and wherein the treatment is a cotreatment with pembrolizumab, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every two weeks and wherein the treatment is a cotreatment with pembrolizumab, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every three weeks and wherein the treatment is a cotreatment with pembrolizumab, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dose is administered to the patient every four weeks and wherein the treatment is a cotreatment with pembrolizumab, which may be given prior to, together with or after administration of the TLR7/8 agonist conjugate and wherein the TLR7/8 agonist is resiquimod.

In certain embodiments the unit dose of the unit dosage form of the first aspect is for use in the treatment of cancer, wherein the unit dosage comprises 0.5 mg TLR7/8 agonist conjugate and is administered to the patient every two to four weeks and wherein the treatment is a cotreatment with pembrolizumab, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every two weeks and wherein the treatment is a cotreatment with pembrolizumab, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every three weeks and wherein the treatment is a cotreatment with pembrolizumab, which may be given prior to, together with or after administration of the TLR7/8 agonist conjugate and wherein the TLR7/8 agonist is resiquimod. In certain embodiments the unit dose of the unit dosage form of the first aspect is for use in the treatment of cancer, wherein the unit dosage comprises 0.5 mg TLR7/8 agonist conjugate and is administered to the patient every four weeks and wherein the treatment is a cotreatment with pembrolizumab, which may be given prior to, together with or after administration of the TLR7/8 agonist conjugate and wherein the TLR7/8 agonist is resiquimod.

The dose, administration frequency and form of administration of pembrolizumab is as described elsewhere herein.

In a second aspect the present invention relates to a TLR7/8 agonist conjugate for use in the treatment of cancer, wherein the TLR7/8 agonist conjugate comprises one or more TLR7/8 agonist moieties reversibly conjugated to a polymeric moiety, wherein the cancer is a solid tumor and wherein the TLR7/8 agonist conjugate is administered via intratumoral administration in a dose ranging from 0.3 mg to 3 mg of TLR7/8 agonist per tumor. In certain embodiments the dose administered to a tumor via intratumoral injection in the second aspect is 0.5 mg of TLR7/8 agonist per tumor. In certain embodiments the dose administered to a tumor via intratumoral injection in the second aspect is 1 mg of TLR7/8 agonist per tumor. In certain embodiments the dose administered to a tumor via intratumoral injection in the second aspect is 2 mg of TLR7/8 agonist per tumor.

In certain embodiments the TLR7/8 agonist conjugate for use of the second aspect is administered intratumorally using a fanning technique.

In certain embodiments the present invention relates to a TLR7/8 agonist conjugate for use in the treatment of cancer, wherein the TLR7/8 agonist conjugate comprises one or more TLR7/8 agonist moieties reversibly conjugated to a polymeric moiety, wherein the cancer is a solid tumor and wherein the TLR7/8 agonist conjugate is administered via intratumoral administration in a dose ranging from 0.3 mg to 3 mg of TLR7/8 agonist per tumor and wherein the TLR7/8 agonist is resiquimod. In certain embodiments the dose administered to a tumor via intratumoral injection in the second aspect is 0.5 mg of TLR7/8 agonist per tumor and wherein the TLR7/8 agonist is resiquimod. In certain embodiments the dose administered to a tumor via intratumoral injection in the second aspect is 1 mg of TLR7/8 agonist per tumor and wherein the TLR7/8 agonist is resiquimod. In certain embodiments the dose administered to a tumor via intratumoral injection in the second aspect is 2 mg of TLR7/8 agonist per tumor and wherein the TLR7/8 agonist is resiquimod.

In certain embodiments the TLR7/8 agonist conjugate for use of the second aspect is administered in a volume ranging from 0.1 ml to 2 ml. In certain embodiments the TLR7/8 agonist conjugate for use of the second aspect is administered in a volume ranging from 0.2 ml to 1.5 ml. In certain embodiments the TLR7/8 agonist conjugate for use of the second aspect is administered in a volume of 0.25 ml. In certain embodiments the TLR7/8 agonist conjugate for use of the second aspect is administered in a volume of 0.5 ml. In certain embodiments the TLR7/8 agonist conjugate for use of the second aspect is administered in a volume of 1 ml.

In certain embodiments the TLR7/8 agonist conjugate for use of the second aspect is administered in a volume ranging from 0.1 ml to 2 ml and the TLR7/8 agonist is resiquimod. In certain embodiments the TLR7/8 agonist conjugate for use of the second aspect is administered in a volume ranging from 0.2 ml to 1.5 ml and the TLR7/8 agonist is resiquimod. In certain embodiments the TLR7/8 agonist conjugate for use of the second aspect is administered in a volume of 0.25 ml and the TLR7/8 agonist is resiquimod. In certain embodiments the TLR7/8 agonist conjugate for use of the second aspect is administered in a volume of 0.5 ml and the TLR7/8 agonist is resiquimod. In certain the TLR7/8 agonist conjugate for use of the second aspect is administered in volume of 1 ml and the TLR7/8 agonist is resiquimod.

In certain embodiments the TLR7/8 agonist conjugate for use of the second aspect is administered in a dose of 0.5 mg of the TLR7/8 agonist in a volume of 0.5 ml. In certain embodiments the TLR7/8 agonist conjugate for use of the second aspect is administered in a dose of 0.5 mg of the TLR7/8 agonist in a volume of 1 ml. In certain embodiments the TLR7/8 agonist conjugate for use of the second aspect is administered in a dose of 0.5 mg of the TLR7/8 agonist in a volume of 0.25 ml. In certain embodiments the TLR7/8 agonist conjugate for use of the second aspect is administered in a dose of 1 mg of the TLR7/8 agonist in a volume of 1 ml. In certain embodiments the TLR7/8 agonist conjugate for use of the second aspect is administered in a dose of 1 mg of the TLR7/8 agonist in a volume of 2 ml. In certain embodiments the TLR7/8 agonist conjugate for use of the second aspect is administered in a dose of 1 mg of the TLR7/8 agonist in a volume of 0.5 ml.

In certain embodiments the TLR7/8 agonist conjugate for use of the second aspect is administered in a dose of 0.5 mg of the TLR7/8 agonist, which TLR7/8 agonist is resiquimod, in a volume of 0.5 ml. In certain embodiments the TLR7/8 agonist conjugate for use of the second aspect is administered in a dose of 0.5 mg of the TLR7/8 agonist, which TLR7/8 agonist is resiquimod, in a volume of 1 ml. In certain embodiments the TLR7/8 agonist conjugate for use of the second aspect is administered in a dose of 0.5 mg of the TLR7/8 agonist, which TLR7/8 agonist is resiquimod, in a volume of 0.25 ml. In certain embodiments the TLR7/8 agonist conjugate for use of the second aspect is administered in a dose of 1 mg of the TLR7/8 agonist, which TLR7/8 agonist is resiquimod, in a volume of 1 ml. In certain embodiments the TLR7/8 agonist conjugate for use of the second aspect is administered in a dose of 1 mg of the TLR7/8 agonist, which TLR7/8 agonist is resiquimod, in a volume of 2 ml. In certain embodiments the TLR7/8 agonist conjugate for use of the second aspect is administered in a dose of 1 mg of the TLR7/8 agonist, which TLR7/8 agonist is resiquimod, in a volume of 0.5 ml.

In certain embodiments the TLR7/8 agonist conjugate for use of the second aspect is provided in a vial comprising the TLR7/8 agonist conjugate or a pharmaceutically acceptable salt thereof. In certain embodiments the TLR7/8 agonist conjugate for use of the second aspect is provided in a vial comprising 0.5 mg of the TLR7/8 agonist conjugate or a pharmaceutically acceptable salt thereof. In certain embodiments the TLR7/8 agonist conjugate for use of the second aspect is provided in a vial comprising 1 mg of the TLR7/8 agonist conjugate or a pharmaceutically acceptable salt thereof. In certain embodiments the TLR7/8 agonist conjugate for use of the second aspect is provided in a vial comprising 2 mg of the TLR7/8 agonist conjugate or a pharmaceutically acceptable salt thereof.

In certain embodiments the TLR7/8 agonist conjugate for use of the second aspect is provided in a vial comprising the TLR7/8 agonist conjugate or a pharmaceutically acceptable salt thereof and one or more excipients. In certain embodiments the TLR7/8 agonist conjugate for use of the second aspect is provided in a vial comprising 0.5 mg of the TLR7/8 agonist conjugate or a pharmaceutically acceptable salt thereof and one or more excipients. In certain embodiments the TLR7/8 agonist conjugate for use of the second aspect is provided in a vial comprising 1 mg of the TLR7/8 agonist conjugate or a pharmaceutically acceptable salt thereof and one or more excipients. In certain embodiments the TLR7/8 agonist conjugate for use of the second aspect is provided in a vial comprising 2 mg of the TLR7/8 agonist conjugate or a pharmaceutically acceptable salt thereof and one or more excipients.

In certain embodiments the TLR7/8 agonist conjugate for use of the second aspect is provided in a vial comprising TLR7/8 agonist conjugate or a pharmaceutically acceptable salt thereof and the content of the vial is lyophilized. In certain embodiments the TLR7/8 agonist conjugate for use of the second aspect is provided in a vial comprising 0.5 mg of the TLR7/8 agonist conjugate or a pharmaceutically acceptable salt thereof and the content of the vial is lyophilized. In certain embodiments the TLR7/8 agonist conjugate for use of the second aspect is provided in a vial comprising 1 mg of the TLR7/8 agonist conjugate or a pharmaceutically acceptable salt thereof and the content of the vial is lyophilized. In certain embodiments the TLR7/8 agonist conjugate for use of the second aspect is provided in a vial comprising 2 mg of the TLR7/8 agonist conjugate or a pharmaceutically acceptable salt thereof and the content of the vial is lyophilized.

In certain embodiments the TLR7/8 agonist conjugate for use of the second aspect is provided in a vial comprising TLR7/8 agonist conjugate or a pharmaceutically acceptable salt thereof and the content of the vial is a liquid, such as a suspension. In certain embodiments the TLR7/8 agonist conjugate for use of the second aspect is provided in a vial comprising 0.5 mg of the TLR7/8 agonist conjugate or a pharmaceutically acceptable salt thereof and the content of the vial is a liquid, such as a suspension. In certain embodiments the TLR7/8 agonist conjugate for use of the second aspect is provided in a vial comprising 1 mg of the TLR7/8 agonist conjugate or a pharmaceutically acceptable salt thereof and the content of the vial is a liquid, such as a suspension. In certain embodiments the TLR7/8 agonist conjugate for use of the second aspect is provided in a vial comprising 2 mg of the TLR7/8 agonist conjugate or a pharmaceutically acceptable salt thereof and the content of the vial is a liquid, such as a suspension.

In certain embodiments the TLR7/8 agonist conjugate for use of the second aspect is provided in a vial comprising TLR7/8 agonist conjugate or a pharmaceutically acceptable salt thereof and one or more excipients and the content of the vial is lyophilized. In certain embodiments the TLR7/8 agonist conjugate for use of the second aspect is provided in a vial comprising 0.5 mg of the TLR7/8 agonist conjugate or a pharmaceutically acceptable salt thereof and one or more excipients and the content of the vial is lyophilized. In certain embodiments the TLR7/8 agonist conjugate for use of the second aspect is provided in a vial comprising 1 mg of the TLR7/8 agonist conjugate or a pharmaceutically acceptable salt thereof and one or more excipients and the content of the vial is lyophilized. In certain embodiments the TLR7/8 agonist conjugate for use of the second aspect is provided in a vial comprising 2 mg of the TLR7/8 agonist conjugate or a pharmaceutically acceptable salt thereof and one or more excipients and the content of the vial is lyophilized.

In certain embodiments the TLR7/8 agonist conjugate for use of the second aspect is provided in a vial comprising TLR7/8 agonist conjugate or a pharmaceutically acceptable salt thereof and one or more excipients and the content of the vial is a liquid, such as a suspension. In certain embodiments the TLR7/8 agonist conjugate for use of the second aspect is provided in a vial comprising 0.5 mg of the TLR7/8 agonist conjugate or a pharmaceutically acceptable salt thereof and one or more excipients and the content of the vial is a liquid, such as a suspension. In certain embodiments the TLR7/8 agonist conjugate for use of the second aspect is provided in a vial comprising 1 mg of the TLR7/8 agonist conjugate or a pharmaceutically acceptable salt thereof and one or more excipients and the content of the vial is a liquid, such as a suspension. In certain embodiments the TLR7/8 agonist conjugate for use of the second aspect is provided in a vial comprising 2 mg of the TLR7/8 agonist conjugate or a pharmaceutically acceptable salt thereof and one or more excipients and the content of the vial is a liquid, such as a suspension.

In certain embodiments the TLR7/8 agonist conjugate for use of the second aspect is provided in a vial comprising the TLR7/8 agonist conjugate or a pharmaceutically acceptable salt thereof, wherein the TLR7/8 agonist is resiquimod. In certain the TLR7/8 agonist conjugate for use of the second aspect is provided in a vial comprising 0.5 mg of the TLR7/8 agonist conjugate or a pharmaceutically acceptable salt thereof, wherein the TLR7/8 agonist is resiquimod. In certain embodiments the TLR7/8 agonist conjugate for use of the second aspect is provided in a vial comprising 1 mg of the TLR7/8 agonist conjugate or a pharmaceutically acceptable salt thereof, wherein the TLR7/8 agonist is resiquimod. In certain embodiments the TLR7/8 agonist conjugate for use of the second aspect is provided in a vial comprising 2 mg of the TLR7/8 agonist conjugate or a pharmaceutically acceptable salt thereof, wherein the TLR7/8 agonist is resiquimod.

In certain embodiments the TLR7/8 agonist conjugate for use of the second aspect is provided in a vial comprising the TLR7/8 agonist conjugate or a pharmaceutically acceptable salt thereof and one or more excipients, wherein the TLR7/8 agonist is resiquimod. In certain embodiments the unit dosage form of the first aspect is a vial comprising 0.5 mg of the TLR7/8 agonist conjugate or a pharmaceutically acceptable salt thereof and one or more excipients, wherein the TLR7/8 agonist is resiquimod. In certain embodiments the TLR7/8 agonist conjugate for use of the second aspect is provided in a vial comprising 1 mg of the TLR7/8 agonist conjugate or a pharmaceutically acceptable salt thereof and one or more excipients, wherein the TLR7/8 agonist is resiquimod. In certain embodiments the TLR7/8 agonist conjugate for use of the second aspect is provided in a vial comprising 2 mg of the TLR7/8 agonist conjugate or a pharmaceutically acceptable salt thereof and one or more excipients, wherein the TLR7/8 agonist is resiquimod.

In certain embodiments the TLR7/8 agonist conjugate for use of the second aspect is provided in a vial comprising the TLR7/8 agonist conjugate or a pharmaceutically acceptable salt thereof, wherein the TLR7/8 agonist is resiquimod and the content of the vial is lyophilized. In certain embodiments the TLR7/8 agonist conjugate for use of the second aspect is provided in a vial comprising 0.5 mg of the TLR7/8 agonist conjugate or a pharmaceutically acceptable salt thereof, wherein the TLR7/8 agonist is resiquimod and the content of the vial is lyophilized. In certain embodiments the TLR7/8 agonist conjugate for use of the second aspect is provided in a vial comprising 1 mg of the TLR7/8 agonist conjugate or a pharmaceutically acceptable salt thereof, wherein the TLR7/8 agonist is resiquimod and the content of the vial is lyophilized. In certain embodiments the TLR7/8 agonist conjugate for use of the second aspect is provided in a vial comprising 2 mg of the TLR7/8 agonist conjugate or a pharmaceutically acceptable salt thereof, wherein the TLR7/8 agonist is resiquimod and the content of the vial is lyophilized.

In certain embodiments the TLR7/8 agonist conjugate for use of the second aspect is provided in a vial comprising the TLR7/8 agonist conjugate or a pharmaceutically acceptable salt thereof, wherein the TLR7/8 agonist is resiquimod and the content of the vial is a liquid, such as a suspension. In certain embodiments the TLR7/8 agonist conjugate for use of the second aspect is provided in a vial comprising 0.5 mg of the TLR7/8 agonist conjugate or a pharmaceutically acceptable salt thereof, wherein the TLR7/8 agonist is resiquimod and the content of the vial is a liquid, such as a suspension. In certain embodiments the TLR7/8 agonist conjugate for use of the second aspect is provided in a vial comprising 1 mg of the TLR7/8 agonist conjugate or a pharmaceutically acceptable salt thereof, wherein the TLR7/8 agonist is resiquimod and the content of the vial is a liquid, such as a suspension. In certain embodiments the TLR7/8 agonist conjugate for use of the second aspect is provided in a vial comprising 2 mg of the TLR7/8 agonist conjugate or a pharmaceutically acceptable salt thereof, wherein the TLR7/8 agonist is resiquimod and the content of the vial is a liquid, such as a suspension.

In certain embodiments the TLR7/8 agonist conjugate for use of the second aspect is provided in a vial comprising the TLR7/8 agonist conjugate or a pharmaceutically acceptable salt thereof and one or more excipients, wherein the TLR7/8 agonist is resiquimod and the content of vial is lyophilized. In certain embodiments the TLR7/8 agonist conjugate for use of the second aspect is provided in a vial comprising 0.5 mg of the TLR7/8 agonist conjugate or a pharmaceutically acceptable salt thereof and one or more excipients, wherein the TLR7/8 agonist is resiquimod and the content of vial is lyophilized. In certain embodiments the TLR7/8 agonist conjugate for use of the second aspect is provided in a vial comprising 1 mg of the TLR7/8 agonist conjugate or a pharmaceutically acceptable salt thereof and one or more excipients, wherein the TLR7/8 agonist is resiquimod and the content of vial is lyophilized. In certain embodiments the TLR7/8 agonist conjugate for use of the second aspect is provided in a vial comprising 2 mg of the TLR7/8 agonist conjugate or a pharmaceutically acceptable salt thereof and one or more excipients, wherein the TLR7/8 agonist is resiquimod and the content of vial is lyophilized.

In certain embodiments the TLR7/8 agonist conjugate for use of the second aspect is provided in a vial comprising the TLR7/8 agonist conjugate or a pharmaceutically acceptable salt thereof and one or more excipients, wherein the TLR7/8 agonist is resiquimod and the content of vial is a liquid, such as a suspension. In certain embodiments the TLR7/8 agonist conjugate for use of the second aspect is provided in a vial comprising 0.5 mg of the TLR7/8 agonist conjugate or a pharmaceutically acceptable salt thereof and one or more excipients, wherein the TLR7/8 agonist is resiquimod and the content of vial is a liquid, such as a suspension. In certain embodiments the TLR7/8 agonist conjugate for use of the second aspect is provided in a vial comprising 1 mg of the TLR7/8 agonist conjugate or a pharmaceutically acceptable salt thereof and one or more excipients, wherein the TLR7/8 agonist is resiquimod and the content of vial is a liquid, such as a suspension. In certain embodiments the TLR7/8 agonist conjugate for use of the second aspect is provided in a vial comprising 2 mg of the TLR7/8 agonist conjugate or a pharmaceutically acceptable salt thereof and one or more excipients, wherein the TLR7/8 agonist is resiquimod and the content of vial is a liquid, such as a suspension.

In certain embodiments the TLR7/8 agonist conjugate for use of the second aspect is provided in a dual-chamber cartridge comprising the TLR7/8 agonist conjugate or a pharmaceutically acceptable salt thereof in a first chamber. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof in a first chamber. In certain embodiments the TLR7/8 agonist conjugate for use of the second aspect is provided in a dual-chamber cartridge comprising 1 mg of the TLR7/8 agonist conjugate or a pharmaceutically acceptable salt thereof in a first chamber. In certain embodiments the TLR7/8 agonist conjugate for use of the second aspect is provided in a dual-chamber cartridge comprising 2 mg of the TLR7/8 agonist conjugate or a pharmaceutically acceptable salt thereof in a first chamber.

In certain embodiments the TLR7/8 agonist conjugate for use of the second aspect is provided in a dual-chamber cartridge comprising the TLR7/8 agonist conjugate or a pharmaceutically acceptable salt thereof and one or more excipients in a first chamber. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof and one or more excipients in a first chamber. In certain embodiments the TLR7/8 agonist conjugate for use of the second aspect is provided in a dual-chamber cartridge comprising 1 mg of the TLR7/8 agonist conjugate or a pharmaceutically acceptable salt thereof and one or more excipients in a first chamber. In certain embodiments the TLR7/8 agonist conjugate for use of the second aspect is provided in a dual-chamber cartridge comprising 2 mg of the TLR7/8 agonist conjugate or a pharmaceutically acceptable salt thereof and one or more excipients in a first chamber.

In certain embodiments the TLR7/8 agonist conjugate for use of the second aspect is provided in a dual-chamber cartridge comprising the TLR7/8 agonist conjugate or a pharmaceutically acceptable salt thereof in the first chamber and wherein the content of said first chamber is lyophilized. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof in the first chamber and wherein the content of said first chamber is lyophilized. In certain embodiments the TLR7/8 agonist conjugate for use of the second aspect is provided in a dual-chamber cartridge comprising 1 mg of the TLR7/8 agonist conjugate or a pharmaceutically acceptable salt thereof in the first chamber and wherein the content of said first chamber is lyophilized. In certain embodiments the TLR7/8 agonist conjugate for use of the second aspect is provided in a dual-chamber cartridge comprising 2 mg of the TLR7/8 agonist conjugate or a pharmaceutically acceptable salt thereof in the first chamber and wherein the content of said first chamber is lyophilized.

In certain embodiments the TLR7/8 agonist conjugate for use of the second aspect is provided in a dual-chamber cartridge comprising the TLR7/8 agonist conjugate or a pharmaceutically acceptable salt thereof and one or more excipients in the first chamber and wherein the content of said first chamber is lyophilized. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof and one or more excipients in the first chamber and wherein the content of said first chamber is lyophilized. In certain embodiments the TLR7/8 agonist conjugate for use of the second aspect is provided in a dual-chamber cartridge comprising 1 mg of the TLR7/8 agonist conjugate or a pharmaceutically acceptable salt thereof and one or more excipients in the first chamber and wherein the content of said first chamber is lyophilized. In certain embodiments the TLR7/8 agonist conjugate for use of the second aspect is provided in a dual-chamber cartridge comprising 2 mg of the TLR7/8 agonist conjugate or a pharmaceutically acceptable salt thereof and one or more excipients in the first chamber and wherein the content of said first chamber is lyophilized.

In certain embodiments the TLR7/8 agonist conjugate for use of the second aspect is provided in a dual-chamber cartridge comprising the TLR7/8 agonist conjugate or a pharmaceutically acceptable salt thereof in a first chamber, wherein the TLR7/8 agonist is resiquimod. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof in a first chamber, wherein the TLR7/8 agonist is resiquimod. In certain embodiments the TLR7/8 agonist conjugate for use of the second aspect is provided in a dual-chamber cartridge comprising 1 mg of the TLR7/8 agonist conjugate or a pharmaceutically acceptable salt thereof in a first chamber, wherein the TLR7/8 agonist is resiquimod. In certain embodiments the TLR7/8 agonist conjugate for use of the second aspect is provided in a dual-chamber cartridge comprising 2 mg of the TLR7/8 agonist conjugate or a pharmaceutically acceptable salt thereof in a first chamber, wherein the TLR7/8 agonist is resiquimod.

In certain embodiments the TLR7/8 agonist conjugate for use of the second aspect is provided in a dual-chamber cartridge comprising the TLR7/8 agonist conjugate or a pharmaceutically acceptable salt thereof and one or more excipients in a first chamber, wherein the TLR7/8 agonist is resiquimod. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof and one or more excipients in a first chamber, wherein the TLR7/8 agonist is resiquimod. In certain embodiments the TLR7/8 agonist conjugate for use of the second aspect is provided in a dual-chamber cartridge comprising 1 mg of the TLR7/8 agonist conjugate or a pharmaceutically acceptable salt thereof and one or more excipients in a first chamber, wherein the TLR7/8 agonist is resiquimod. In certain embodiments the TLR7/8 agonist conjugate for use of the second aspect is provided in a dual-chamber cartridge comprising 2 mg of the TLR7/8 agonist conjugate or a pharmaceutically acceptable salt thereof and one or more excipients in a first chamber, wherein the TLR7/8 agonist is resiquimod.

In certain embodiments the TLR7/8 agonist conjugate for use of the second aspect is provided in a dual-chamber cartridge comprising the TLR7/8 agonist conjugate or a pharmaceutically acceptable salt thereof in the first chamber, wherein the TLR7/8 agonist is resiquimod and wherein the content of said first chamber is lyophilized. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof in the first chamber, wherein the TLR7/8 agonist is resiquimod and wherein the content of said first chamber is lyophilized. In certain embodiments the TLR7/8 agonist conjugate for use of the second aspect is provided in a dual-chamber cartridge comprising 1 mg of the TLR7/8 agonist conjugate or a pharmaceutically acceptable salt thereof in the first chamber, wherein the TLR7/8 agonist is resiquimod and wherein the content of said first chamber is lyophilized. In certain embodiments the TLR7/8 agonist conjugate for use of the second aspect is provided in a dual-chamber cartridge comprising 2 mg of the TLR7/8 agonist conjugate or a pharmaceutically acceptable salt thereof in the first chamber, wherein the TLR7/8 agonist is resiquimod and wherein the content of said first chamber is lyophilized.

In certain embodiments the TLR7/8 agonist conjugate for use of the second aspect is provided in a dual-chamber cartridge comprising the TLR7/8 agonist conjugate or a pharmaceutically acceptable salt thereof and one or more excipients in the first chamber, wherein the TLR7/8 agonist is resiquimod and wherein the content of said first chamber is lyophilized. In certain embodiments the TLR7/8 agonist conjugate for use of the second aspect is provided in a dual-chamber cartridge comprising 0.5 mg of the TLR7/8 agonist conjugate or a pharmaceutically acceptable salt thereof and one or more excipients in the first chamber, wherein the TLR7/8 agonist is resiquimod and wherein the content of said first chamber is lyophilized. In certain embodiments the TLR7/8 agonist conjugate for use of the second aspect is provided in a dual-chamber cartridge comprising 1 mg of the TLR7/8 agonist conjugate or a pharmaceutically acceptable salt thereof and one or more excipients in the first chamber, wherein the TLR7/8 agonist is resiquimod and wherein the content of said first chamber is lyophilized. In certain embodiments the TLR7/8 agonist conjugate for use of the second aspect is provided in a dual-chamber cartridge comprising 2 mg of the TLR7/8 agonist conjugate or a pharmaceutically acceptable salt thereof and one or more excipients in the first chamber, wherein the TLR7/8 agonist is resiquimod and wherein the content of said first chamber is lyophilized.

In all embodiments the second chamber of a dual-chamber cartridge may comprise buffer or water for resuspension.

The TLR7/8 agonist conjugate for use of the second aspect is in certain embodiments for use in the treatment of a solid tumor. In certain embodiment TLR7/8 agonist of the second aspect is administered via intratumoral injection, such as by using a fanning technique. In certain embodiments the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma. In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC).

In certain embodiments the TLR7/8 agonist for use of the second aspect is administered to the patient every two to four weeks. In certain embodiments the TLR7/8 agonist for use of the second aspect is administered to the patient every two weeks. In certain embodiments the TLR7/8 agonist for use of the second aspect is administered to the patient every three weeks. In certain embodiments the TLR7/8 agonist for use of the second aspect is administered to the patient every four weeks.

In certain embodiments the TLR7/8 agonist for use of the second aspect is administered to the patient every two to four weeks and the treatment is a cotreatment with an immune checkpoint inhibitor, which may be given prior to, together with or after administration of the TLR7/8 agonist conjugate. In certain embodiments the TLR7/8 agonist for use of the second aspect is administered to the patient every two weeks and the treatment is a cotreatment with an immune checkpoint inhibitor, which may be given prior to, together with or after administration of the TLR7/8 agonist conjugate. In certain embodiments the TLR7/8 agonist for use of the second aspect is administered to the patient every three weeks and the treatment is a cotreatment with an immune checkpoint inhibitor, which may be given prior to, together with or after administration of the TLR7/8 agonist conjugate. In certain embodiments the TLR7/8 agonist for use of the second aspect is administered to the patient every four weeks and the treatment is a cotreatment with an immune checkpoint inhibitor, which may be given prior to, together with or after administration of the TLR7/8 agonist conjugate.

In certain embodiments the TLR7/8 agonist for use of the second aspect is administered to the patient every two to four weeks and wherein the treatment is a cotreatment with an inhibitor of PD-1 or an inhibitor of PD-L1, which may be given prior to, together with or after administration of the TLR7/8 agonist conjugate. In certain embodiments the TLR7/8 agonist for use of the second aspect is administered to the patient every two weeks and wherein the treatment is a cotreatment with an inhibitor of PD-1 or an inhibitor of PD-L1, which may be given prior to, together with or after administration of the TLR7/8 agonist conjugate. In certain embodiments the TLR7/8 agonist for use of the second aspect is administered to the patient every three weeks and wherein the treatment is a cotreatment with an inhibitor of PD-1 or an inhibitor of PD-L1, which may be given prior to, together with or after administration of the TLR7/8 agonist conjugate. In certain embodiments the TLR7/8 agonist for use of the second aspect is administered to the patient every four weeks and wherein the treatment is a cotreatment with an inhibitor of PD-1 or an inhibitor of PD-L1, which may be given prior to, together with or after administration of the TLR7/8 agonist conjugate.

An inhibitor of PD-1 (programmed cell death protein 1) may be selected from the group consisting of pembrolizumab, nivolumab, pidilizumab, AMP-224, BMS-936559, cemiplimab and PDR001. An inhibitor of PD-L1 (programmed cell death ligand 1) may be selected from the group consisting of MDX-1105, MEDI4736, atezolizumab, avelumab, BMS-936559 and durvalumab.

In certain embodiments the TLR7/8 agonist for use of the second aspect is administered to the patient every two to four weeks and wherein the treatment is a cotreatment with an inhibitor of PD-1, which may be given prior to, together with or after administration of the TLR7/8 agonist conjugate. In certain embodiments the TLR7/8 agonist for use of the second aspect is administered to the patient every two weeks and wherein the treatment is a cotreatment with an inhibitor of PD-1, which may be given prior to, together with or after administration of the TLR7/8 agonist conjugate. In certain the TLR7/8 agonist for use of the second aspect is administered to the patient every three weeks and wherein the treatment is a cotreatment with an inhibitor of PD-1, which may be given prior to, together with or after administration of the TLR7/8 agonist conjugate. In certain embodiments the TLR7/8 agonist for use of the second aspect is administered to the patient every four weeks and wherein the treatment is a cotreatment with an inhibitor of PD-1, which may be given prior to, together with or after administration of the TLR7/8 agonist conjugate.

In certain embodiments the TLR7/8 agonist for use of the second aspect is administered to the patient every two to four weeks and wherein the treatment is a cotreatment with an inhibitor of PD-L1, which may be given prior to, together with or after administration of the TLR7/8 agonist conjugate. In certain embodiments the TLR7/8 agonist for use of the second aspect is administered to the patient every two weeks and wherein the treatment is a cotreatment with an inhibitor of PD-L1, which may be given prior to, together with or after administration of the TLR7/8 agonist conjugate. In certain embodiments the TLR7/8 agonist for use of the second aspect is administered to the patient every three weeks and wherein the treatment is a cotreatment with an inhibitor of PD-L1, which may be given prior to, together with or after administration of the TLR7/8 agonist conjugate. In certain embodiments the TLR7/8 agonist for use of the second aspect is administered to the patient every four weeks and wherein the treatment is a cotreatment with an inhibitor of PD-L1, which may be given prior to, together with or after administration of the TLR7/8 agonist conjugate.

In certain embodiments the TLR7/8 agonist for use of the second aspect is administered to the patient every two to four weeks and wherein the treatment is a cotreatment with pembrolizumab, which may be given prior to, together with or after administration of the TLR7/8 agonist conjugate. In certain embodiments the TLR7/8 agonist for use of the second aspect is administered to the patient every two weeks and wherein the treatment is a cotreatment with pembrolizumab, which may be given prior to, together with or after administration of the TLR7/8 agonist conjugate. In certain embodiments the TLR7/8 agonist for use of the second aspect is administered to the patient every three weeks and wherein the treatment is a cotreatment with pembrolizumab, which may be given prior to, together with or after administration of the TLR7/8 agonist conjugate. In certain embodiments the TLR7/8 agonist for use of the second aspect is administered to the patient every four weeks and wherein the treatment is a cotreatment with pembrolizumab, which may be given prior to, together with or after administration of the TLR7/8 agonist conjugate.

The dose, administration frequency and form of administration of pembrolizumab is as described elsewhere herein.

In a third aspect the present invention relates to a method of treating cancer, the method comprising the step of administering to a patient in need thereof a pharmaceutically effective dose of a TLR7/8 agonist conjugate, wherein the TLR7/8 agonist conjugate comprises one or more TLR 7/8 agonist moieties reversibly conjugated to a polymeric moiety and wherein the pharmaceutically effective dose ranges from 0.3 mg to 3 mg of TLR7/8 agonist. In certain embodiments the pharmaceutically effective dose is 0.5 mg TLR7/8 agonist. In certain embodiments the pharmaceutically effective dose is 1 mg.

In certain embodiments the method of the third aspect comprises the step of intratumorally administering the pharmaceutically effective dose, which ranges from 0.3 to 3 mg of TLR7/8 agonist per tumor. In certain embodiments the effective dose is 0.5 mg TLR7/8 agonist per tumor. In certain embodiments the effective dose is 1 mg per tumor.

In certain embodiments the TLR7/8 agonist of the third aspect is resiquimod.

In certain embodiments the intratumoral administration is an intratumoral injection. In certain embodiments such intratumoral injection uses a fanning technique.

In certain embodiments the pharmaceutically effective dose of the third aspect is provided in a volume ranging from 0.1 ml to 2 ml. In certain embodiments the pharmaceutically effective dose of the third aspect is provided in a volume ranging from 0.2 ml to 1.5 ml. In certain embodiments the pharmaceutically effective dose of the third aspect is provided in a volume of 0.25 ml. In certain the pharmaceutically effective dose of the third aspect is provided in a volume of 0.5 ml. In certain embodiments the pharmaceutically effective dose of the third aspect is provided in a volume of 1 ml.

In certain embodiments the pharmaceutically effective dose of the third aspect is provided in a vial. In certain embodiments the pharmaceutically effective dose of the third aspect is provided in a dual-chamber cartridge.

In certain embodiments the pharmaceutically effective dose of the third aspect is provided in the form of a pharmaceutical formulation. In certain embodiments such pharmaceutical formulation is provided as a dry formulation, such as in the form of a lyophilized formulation. Such lyophilized formulation is resuspended before administration to the patient to yield a resuspended formulation, which may be a solution or suspension. In certain embodiments the pharmaceutical formulation is provided as a liquid, such as a suspension formulation. In certain embodiments the pharmaceutical formulation is provided as a suspension formulation. In certain embodiments the pharmaceutical formulation is provided as a suspension formulation which is stored at a temperature ranging from −80° C. to 12° C. In certain embodiments the pharmaceutical formulation is provided as a suspension formulation which is stored at a temperature of about −20° C.

In certain embodiments the cancer of the third aspect is a solid tumor. In certain embodiments the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma. In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC).

In certain embodiments the pharmaceutically effective dose of the third aspect is administered to the patient every two to four weeks. In certain embodiments the pharmaceutically effective dose of the third aspect is administered to the patient every two weeks. In certain embodiments the pharmaceutically effective dose of the third aspect is administered to the patient every three weeks. In certain embodiments the pharmaceutically effective dose of the third aspect is administered to the patient every four weeks.

In certain embodiments the pharmaceutically effective dose of the third aspect is administered to the patient every two to four weeks and the treatment is a cotreatment with an immune checkpoint inhibitor, which may be given prior to, together with or after administration of the TLR7/8 agonist conjugate. In certain embodiments the pharmaceutically effective dose of the third aspect is administered to the patient every two weeks and the treatment is a cotreatment with an immune checkpoint inhibitor, which may be given prior to, together with or after administration of the TLR7/8 agonist conjugate. In certain the pharmaceutically effective dose of the third aspect is administered to the patient every three weeks and the treatment is a cotreatment with an immune checkpoint inhibitor, which may be given prior to, together with or after administration of the TLR7/8 agonist conjugate. In certain the pharmaceutically effective dose of the third aspect is administered to the patient every four weeks and the treatment is a cotreatment with an immune checkpoint inhibitor, which may be given prior to, together with or after administration of the TLR7/8 agonist conjugate.

In certain embodiments the immune checkpoint inhibitor of the third aspect is an inhibitor of PD-1 or an inhibitor of PD-L1. An inhibitor of PD-1 (programmed cell death protein 1) may be selected from the group consisting of pembrolizumab, nivolumab, pidilizumab, AMP-224, BMS-936559, cemiplimab and PDR001. An inhibitor of PD-L1 (programmed cell death ligand 1) may be selected from the group consisting of MDX-1105, MEDI4736, atezolizumab, avelumab, BMS-936559 and durvalumab.

In certain embodiments the immune checkpoint inhibitor is an inhibitor of PD-1. In certain embodiments the immune checkpoint inhibitor is pembrolizumab.

In the following sections the TLR7/8 agonist conjugate of all aspects of the present invention will be described in further detail.

The TLR7/8 agonist conjugate comprises a polymeric moiety Z to which one or more moieties -L²-L¹-D are conjugated, wherein each -L²- is individually a chemical bond or a spacer moiety; each -L¹- is individually a linker moiety to which -D is reversibly and covalently conjugated; and each -D is a TLR7/8 agonist.

The one or more moieties -L²-L¹-D are covalently conjugated to Z. In certain embodiments the one or more moieties -L²-L¹-D are stably conjugated to Z. If Z is a hydrogel it is understood that the number of moieties -L²-L¹-D conjugated to such hydrogel carrier is too large to specify.

In certain embodiments -D is a TLR7/8 agonist selected from the group consisting of CL075, CL097, poly(dT), resiquimod (R-848, VML600, S28463), MEDI9197 (3M-052), NKTR262, DV1001, IMO4200, IPH3201 and VTX1463.

In certain embodiments -D is resiquimod.

In certain embodiments at least some moieties-D of the conjugate are resiquimod, such as about 10%, about 20%, about 30%, about 40%, about 50%, about 60%, about 70%, about 80%, about 90% or 100%, i.e. all, of the moieties-D present in the conjugate.

In certain embodiments the conjugate comprises only one type of moiety-D, i.e. all moieties-D of the conjugate are identical. In certain embodiments the conjugate comprises more than one type of -D, such as 2, 3, 4, 5, 6, 7, 8, 9 or 10 different types of -D.

If the conjugate comprises more than one type of -D, all moieties-D may be conjugated to the same type of -L¹- or may be conjugated to different types of -L¹-, i.e. a first type of -D may be conjugated to a first type of -L¹-, a second type of -D may be conjugated to a second type -L¹-, and so on. In certain embodiments all moieties -L¹- are of the same type, i.e. have the same structure. Alternatively, individual moieties-D of the same type may be conjugated to different types of moiety -L¹-. The use of different moieties -L¹- allows for release of the conjugated drug moieties-D with different release kinetics. For example, a first linker moiety -L¹- may have a short half-life and thus provides drug release within a shorter time after administration to a patient than a second linker moiety -L¹- which may have a longer half-life. Using different moieties -L¹- with different release half-lives allows for an optimized dosage regimen of one or more drugs.

The moiety -L¹- is conjugated to -D via a functional group of -D, which functional group is in certain embodiments selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, aziridine, amide, imide, imine, urea, amidine, guanidine, sulfonamide, phosphonamide, phorphoramide, hydrazide and selenol. In certain embodiments -L¹- is conjugated to -D via a functional group of -D selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, amidine and aziridine. In certain embodiments -L¹- is conjugated to -D via a functional group of -D selected from the group consisting of hydroxyl, primary amine, secondary amine, amidine and carboxylic acid.

In certain embodiments -L¹- is conjugated to -D via a hydroxyl group of -D.

In certain embodiments -L¹- is conjugated to -D via a primary amine group of -D.

In certain embodiments -L¹- is conjugated to -D via a secondary amine group of -D.

In certain embodiments -L¹- is conjugated to -D via a carboxylic acid group of -D.

In certain embodiments -L¹- is conjugated to -D via an amidine group of -D.

If -D is resiquimod, -L¹- is in certain embodiments conjugated to -D via its aromatic amine, i.e. the amine functional group marked with the asterisk

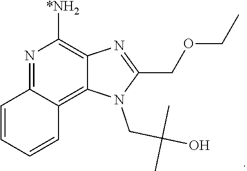

The moiety -L¹- can be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide, acylguanidine, acylamidine, carbonate, phosphate, sulfate, urea, hydrazide, thioester, thiophosphate, thiosulfate, sulfonamide, sulfoamidine, sulfaguanidine, phosphoramide, phosphoamidine, phosphoguanidine, phosphonamide, phosphonamidine, phosphonguanidine, phosphonate, borate and imide. In certain embodiments -L¹- is connected to -D through a linkage selected from the group consisting of amide, ester, carbonate, carbamate, acetal, aminal, imine, oxime, hydrazone, disulfide, acylamidine and acylguanidine. In certain embodiments -L¹- is connected to -D through a linkage selected from the group consisting of amide, ester, caronate, acylamide and carbamate. It is understood that some of these linkages may not be reversible per se, but that in the present invention neighboring groups present in -L¹- render these linkages reversible.

In certain embodiments -L¹- is connected to -D through an ester linkage.

In certain embodiments -L¹- is connected to -D through a carbonate linkage.

In certain embodiments -L¹- is connected to -D through an acylamidine linkage.

In certain embodiments -L¹- is connected to -D through a carbamate linkage.

In certain embodiments -L¹- is connected to -D through an amide linkage.

If -D is resiquimod, the linkage between-D and -L¹- is in certain embodiments through an amide linkage, in which the aromatic amine functional group of -D forms an amide linkage with a carbonyl (—(C═O)—) of -L¹-.

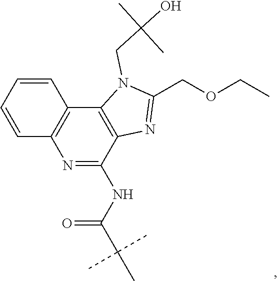

wherein the dashed line indicates attachment to the remainder of -L¹-.

In certain embodiments cleavage of the linkage between -D and -L¹- occurs with a release half-life under physiological conditions (aqueous buffer, pH 7.4, 37° C.) of at least 3 days, such as at least 4 days, at least 5 days, at least 6 days, at least 7 days, at least 8 days, at least 9 days, at least 10 days, at least 12 days, at least 15 days, at least 17 days, at least 20 days or at least 25 days.

The moiety -L¹- is a linker moiety from which -D is released in its free form, i.e. generally in the form of D-H or D-OH. Such moieties are also known as “prodrug linkers” or “reversible prodrug linkers” and are known in the art, such as for example the reversible linker moieties disclosed in WO 2005/099768 A2, WO 2006/136586 A2, WO 2011/089216 A1, WO 2013/024053 A1, WO 2011/012722 A1, WO 2011/089214 A1, WO 2011/089215 A1, WO 2013/024052 A1 and WO 2013/160340 A1, which are incorporated by reference herewith.

In one embodiment -L¹- has a structure as disclosed in WO 2009/095479 A2. Accordingly, in certain embodiments the moiety -L¹- is of formula (II):

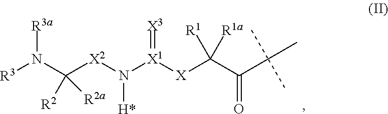

wherein the dashed line indicates attachment to a nitrogen of -D by forming an amide bond;
—X— is —C(R⁴R^{4a})—; —N(R⁴)—; —O—; —C(R⁴R^{4a})—C(R⁵R^{5a})—; —C(R⁵R^{5a})—C(R⁴R^{4a})—; —C(R⁴R^{4a})—N(R⁶)—; —N(R⁶)—C(R⁴R^{4a})—; —C(R⁴R^{4a})—O—; —O—C(R⁴R^{4a})—; or —C(R⁷R^{7a})—;
X¹ is C; or S(O);
—X²— is —C(R⁸R^{8a})—; or —C(R⁸R^{8a})—C(R⁹R^{9a})—;
═X³ is ═O; ═S; or ═N—CN;
—R¹, —R^{1a}, —R², —R^{2a}, —R⁴, —R^{4a}, —R⁵, —R^{5a}, —R⁶, —R⁸, —R^{8a}, —R⁹, —R^{9a} are independently selected from the group consisting of —H; and C₁₋₆ alkyl;
—R³, —R^{3a} are independently selected from the group consisting of —H; and C₁₋₆ alkyl, provided that in case one of —R³, —R^{3a} or both are other than-H they are connected to N to which they are attached through an sp³-hybridized carbon atom;
—R⁷ is —N(R¹⁰R^{10a}); or —NR¹⁰—(C═O)—R¹¹;
—R^{7a}, —R¹⁰, —R^{10a}, —R¹¹ are independently of each other-H; or C₁₋₆ alkyl;
optionally, one or more of the pairs —R^{1a}/—R^{4a}, —R^{1a}/—R^{5a}, —R^{1a}/—R^{7a}, —R^{4a}/—R^{5a}, —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a}, —R⁹/—R^{9a} are joined together with the atom to which they are attached to form a C₃₋₁₀ cycloalkyl; or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹, —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with at least one -L²- and wherein -L¹- is optionally further substituted, provided that the hydrogen marked with the asterisk in formula (II) is not replaced by -L²- or a substituent.

Preferably -L¹- of formula (II) is substituted with one moiety -L²-.

In one embodiment -L¹- of formula (II) is not further substituted.

It is understood that if —R³/—R^{3a} of formula (II) are joined together with the nitrogen atom to which they are attached to form a 3- to 10-membered heterocycle, only such 3- to 10-membered heterocycles may be formed in which the atoms directly attached to the nitrogen are sp³-hybridized carbon atoms. In other words, such 3- to 10-membered heterocycle formed by —R³/—R^{3a} together with the nitrogen atom to which they are attached has the following structure:

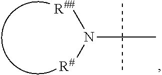

wherein
the dashed line indicates attachment to the rest of -L¹-;
the ring comprises 3 to 10 atoms comprising at least one nitrogen; and
R^{#} and R^{##} represent an sp³-hydridized carbon atom.

It is also understood that the 3- to 10-membered heterocycle may be further substituted.

Exemplary embodiments of suitable 3- to 10-membered heterocycles formed by —R³/—R^{3a} of formula (II) together with the nitrogen atom to which they are attached are the following:

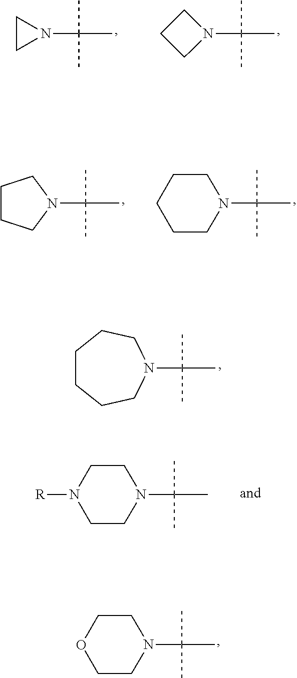

wherein
dashed lines indicate attachment to the rest of the molecule; and
—R is selected from the group consisting of —H and C₁₋₆ alkyl.

-L¹- of formula (II) may optionally be further substituted. In general, any substituent may be used as far as the cleavage principle is not affected, i.e. the hydrogen marked with the asterisk in formula (II) is not replaced and the nitrogen of the moiety

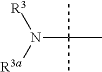

of formula (II) remains part of a primary, secondary or tertiary amine, i.e. —R³ and —R^{3a} are independently of each other —H or are connected to —N<through an sp³-hybridized carbon atom.

In one embodiment —R¹ or —R^{1a} of formula (II) is substituted with -L²-. In another embodiment —R² or —R^{2a} of formula (II) is substituted with -L²-. In another embodiment —R³ or —R^{3a} of formula (II) is substituted with -L²-. In another embodiment —R⁴ of formula (II) is substituted with -L²-. In another embodiment —R⁵ or —R^{5a} of formula (II) is substituted with -L²-. In another embodiment —R⁶ of formula (II) is substituted with -L²-. In another embodiment —R⁷ or —R^{7a} of formula (II) is substituted with -L²-. In another embodiment —R⁸ or —R^{8a} of formula (II) is substituted with -L²-. In another embodiment —R⁹ or —R^{9a} of formula (II) is substituted with -L²-. In another embodiment —R¹⁰ or —R^{10a} of formula (II) is substituted with -L²-. In another embodiment —R¹¹ of formula (II) is substituted with -L²-.

In certain embodiments -L¹- has a structure as disclosed in WO2016/020373A1. Accordingly, in certain embodiments the moiety -L¹- is of formula (III):

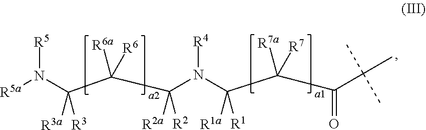

wherein
the dashed line indicates attachment to a primary or secondary amine or hydroxyl of -D by forming an amide or ester linkage, respectively;
—R¹, —R^{1a}, —R², —R^{2a}, —R³ and —R^{3a} are independently of each other selected from the group consisting
of —H, —C(R⁸R^{8a}R^{8b}), —C(═O)R⁸, —C═N, —C(═NR⁸)R^{8a}, —CR⁸(═CR^{8a}R^{8b}), —C≡CR⁸ and -T; —R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O)N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹/—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joint together with the atoms to which they are attached to form a ring A;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl;
wherein -L¹- is substituted with at least one -L²- and wherein -L¹- is optionally further substituted.

The optional further substituents of -L¹- of formula (III) are preferably as described above.

Preferably -L¹- of formula (III) is substituted with one moiety -L²-.

In one embodiment -L¹- of formula (III) is not further substituted.

In another embodiment -L¹- has a structure as disclosed in EP1536334B1, WO2009/009712A1, WO2008/034122A1, WO2009/143412A2, WO2011/082368A2, and U.S. Pat. No. 8,618,124B2, which are herewith incorporated by reference.

In another embodiment -L¹- has a structure as disclosed in U.S. Pat. No. 8,946,405B2 and U.S. Pat. No. 8,754,190B2, which are herewith incorporated by reference. Accordingly, in certain embodiments -L¹- is of formula (IV):

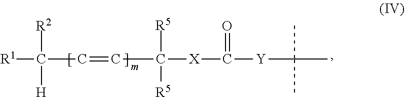

wherein
the dashed line indicates attachment to -D through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally alkenyl, substituted optionally substituted alkynyl, —C(O)R³, —S(O)R³, —S(O)₂R³, and —SR₄,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and
optionally, both —R⁹ together with the nitrogen to which they are attached form a heterocyclic ring;
wherein -L¹- is substituted with at least one -L²- and wherein -L¹- is optionally further substituted.

Only in the context of formula (IV) the terms used have the following meaning:

The term “alkyl” as used herein includes linear, branched or cyclic saturated hydrocarbon groups of 1 to 8 carbons, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR², —OCOR, —NRCOR, —COOR, —CONR₂, —SOR, —SO₂R, —SONR₂, —SO₂N R₂, wherein each R is independently alkyl, alkenyl, alkynyl, aryl or heteroaryl, or two R groups taken together with the atoms to which they are attached form a ring.

Preferably -L¹- of formula (IV) is substituted with one moiety -L²-.

In another embodiment -L¹- has a structure as disclosed in WO2013/036857A1, which is herewith incorporated by reference. Accordingly, in certain embodiments -L¹- is of formula (V):

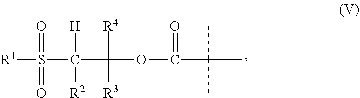

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H;
optionally substituted C₁-C₆ alkyl; optionally substituted aryl; and optionally substituted heteroaryl; or when taken together two —R⁵ can be cycloalkyl or cycloheteroalkyl;
wherein -L¹- is substituted with at least one -L²- and wherein -L¹- is optionally further substituted.

Only in the context of formula (V) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, preferably 6-10 carbons, including groups such as phenyl, naphthyl, and anthracene. “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketene; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

Preferably -L¹- of formula (V) is substituted with one moiety -L²-.

In another embodiment -L¹- has a structure as disclosed in U.S. Pat. No. 7,585,837B2, which is herewith incorporated by reference. Accordingly, in certain embodiments -L¹- is of formula (VI):

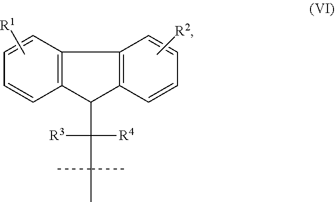

wherein
the dashed line indicates attachment to -D through an amine functional group of -D;
R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl;
wherein -L¹- is substituted with at least one -L²- and wherein -L¹- is optionally further substituted.

Suitable substituents for formulas (VI) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (VI) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

Preferably -L¹- of formula (VI) is substituted with one moiety -L²-.

In another embodiment -L¹- has a structure as disclosed in WO2002/089789A1, which is herewith incorporated by reference. Accordingly, in certain embodiments -L¹- is of formula (VII):

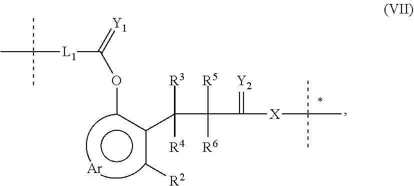

wherein
the dashed line indicates attachment to -D through an amine functional group of -D;
L₁ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁-6 heteroalkyls, C₁₋₆ alkoxy, phenoxy, and C₁₋₆ heteroalkoxy;
Ar is a moiety which when included in formula (VII) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1;
wherein -L¹- is substituted with at least one -L²- and wherein -L¹- is optionally further substituted.

Only in the context of formula (VII) the terms used have the following meaning:

The term “alkyl” shall be understood to include, e.g. straight, branched, substituted C₁-12 alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moeities such as methoxy; and phenoxy includes moieties such as 3-nitrophenoxy. Halo-shall be understood to include fluoro, chloro, iodo and bromo.

Preferably -L¹- of formula (VII) is substituted with one moiety -L²-.

In certain embodiments -L¹-comprises a substructure of formula (VIII)

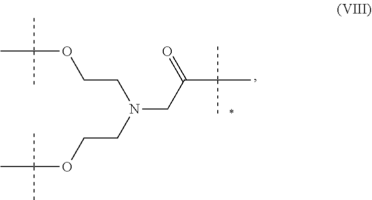

wherein
the dashed line marked with the asterisk indicates attachment to a nitrogen of -D by forming an amide bond;
the unmarked dashed lines indicate attachment to the remainder of -L¹-; and
wherein -L¹- is substituted with at least one -L²- and wherein -L¹- is optionally further substituted.

Preferably -L¹- of formula (VIII) is substituted with one moiety -L²-.

In one embodiment -L¹- of formula (VIII) is not further substituted.

In certain embodiments -L¹-comprises a substructure of formula (IX)

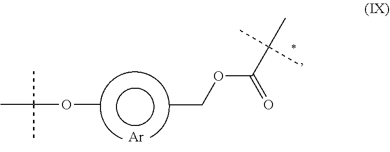

wherein
the dashed line marked with the asterisk indicates attachment to a nitrogen of -D by forming a carbamate bond;
the unmarked dashed lines indicate attachment to the remainder of -L¹-; and
wherein -L¹- is substituted with at least one -L²- and wherein -L¹- is optionally further substituted.

Preferably -L¹- of formula (IX) is substituted with one moiety -L²-.

In one embodiment -L¹- of formula (IX) is not further substituted.

In certain embodiments -L¹- is of formula (IX-a):

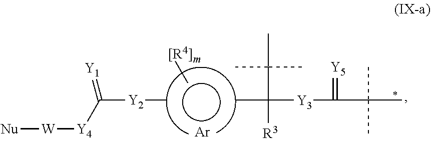

wherein
the dashed line marked with the asterisk indicates attachment to a nitrogen of -D and the unmarked dashed line indicates attachment to -L²-;
n is 0, 1, 2, 3, or 4;
═Y₁, ═Y₅ are independently of each other selected from the group consisting of ═O and ═S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})—;
—R³, —R⁵, —R⁶, —R⁶ are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
-Nu is a nucleophile selected from the group consisting of —N(R⁷R^{7a}), —N(R⁷OH), —N(R⁷)—N(R^{7a}R^{7b}), —S(R⁷), —COOH,

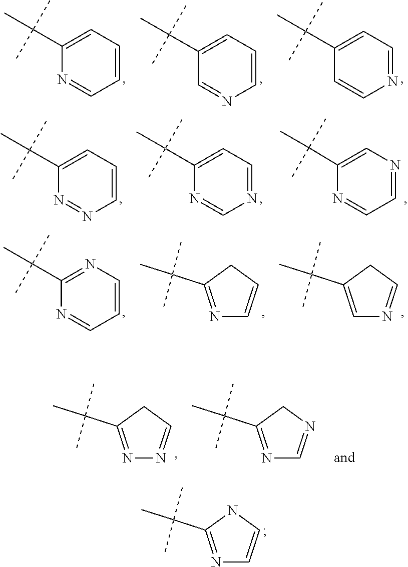

—Ar— is selected from the group consisting of

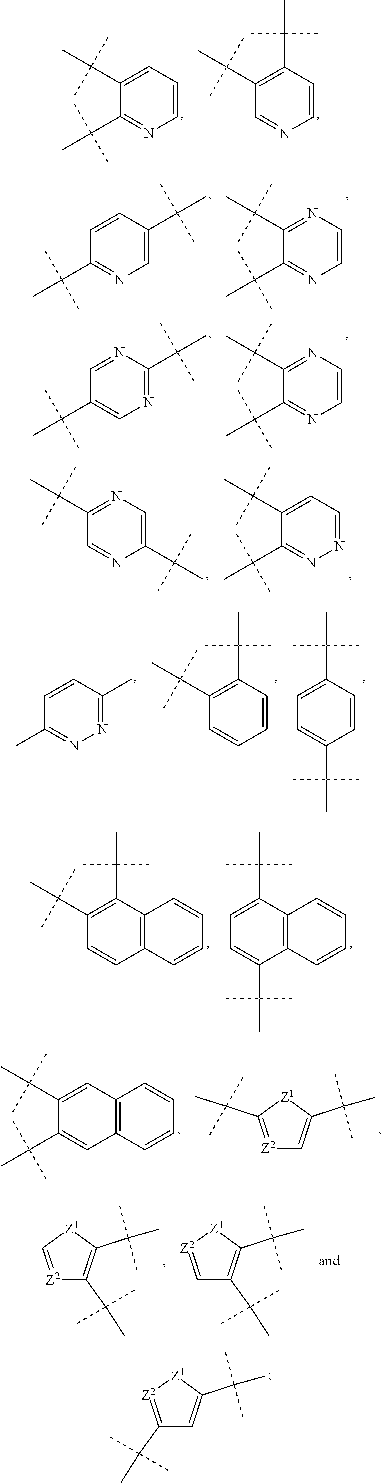

wherein
dashed lines indicate attachment to the remainder of -L¹-, —Z¹— is selected from the group consisting of —O—, —S— and —N(R⁷)—, and
—Z— is —N(R⁷)—; and
—R⁷, —R^{7a}, —R^{7b} are independently of each other selected from the group consisting of —H, C₁₋₆ alkyl, C₂₋₆ alkenyl and C₂₋₄ alkynyl;
wherein -L¹- is optionally further substituted.

In one embodiment -L¹- of formula (IX-a) is not further substituted.

In certain embodiments -L¹- is of formula (IX-b):

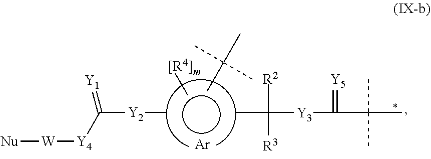

wherein
the dashed line marked with the asterisk indicates attachment to a nitrogen of -D and the unmarked dashed line indicates attachment to -L²-;
n is 0, 1, 2, 3, or 4;
═Y₁, ═Y₅ are independently of each other selected from the group consisting of ═O and ═S;
—Y₂— is selected from the group consisting of —O— and —S—;
—Y₃— is selected from the group consisting of —O— and —S—;
—Y₄— is selected from the group consisting of —O—, —NR⁵— and —C(R⁶R^{6a})—;
—R², —R³, —R⁵, —R⁶, —R^{6a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—R⁴ is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
—W— is selected from the group consisting of C₁₋₂₀ alkyl optionally interrupted by one or more groups selected from the group consisting of C₃₋₁₀ cycloalkyl, 8- to 30-membered carbopolycyclyl, 3- to 10-membered heterocyclyl, —C(O)—, —C(O)N(R⁷)—, —O—, —S— and —N(R⁷)—;
-Nu is a nucleophile selected from the group consisting of —N(R⁷R^{7a}), —N(R⁷OH), —N(R⁷)—N(R^{7a}R^{7b}), —S(R⁷), —COOH,

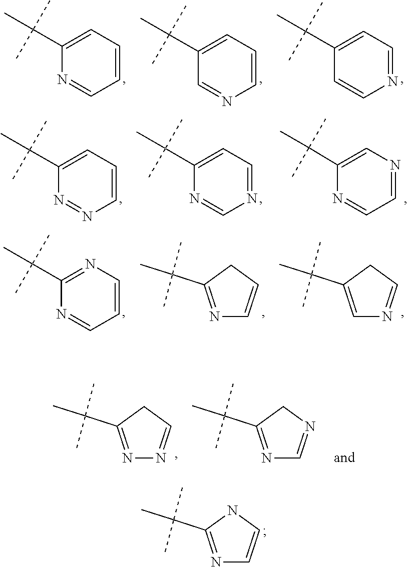

—Ar— is selected from the group consisting of

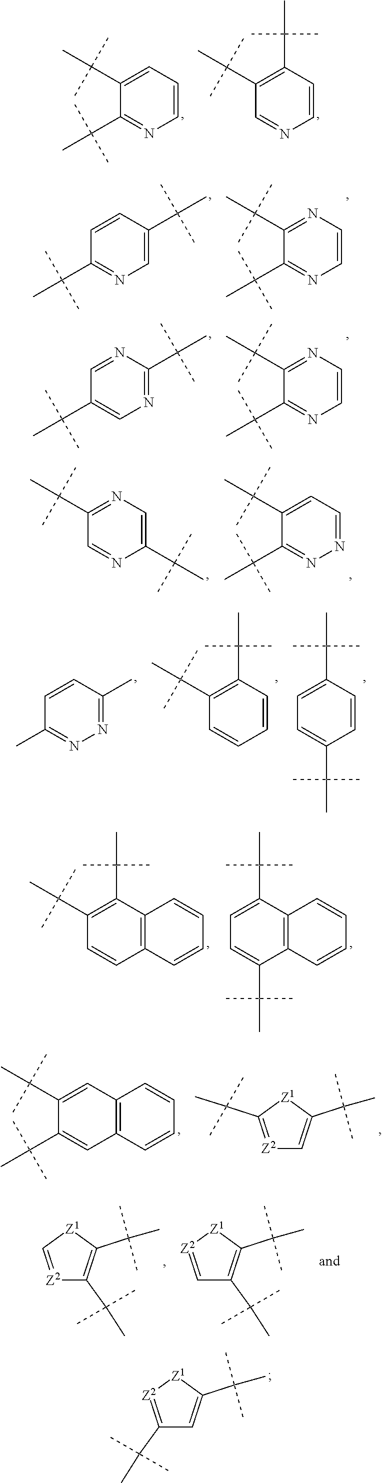

wherein
dashed lines indicate attachment to the remainder of -L¹-, —Z¹— is selected from the group consisting of —O—, —S— and —N(R⁷)—, and
—Z²— is —N(R⁷)—; and
—R⁷, —R^{7a}, —R⁷⁰ are independently of each other selected from the group consisting of —H, C₁₋₆ alkyl, C₂₋₆ alkenyl and C₂₋₆ alkynyl;
wherein -L¹- is optionally further substituted.

In one embodiment -L¹- of formula (IX-b) is not further substituted.

In certain embodiments -L¹- is of formula (X)

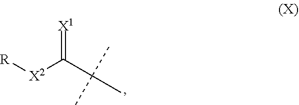

wherein
the dashed line indicates attachment to a nitrogen of an amine functional group of -D;
═X¹ is selected from the group consisting of ═O, ═S and ═N;
—X²— is selected from the group consisting of —O—, —S— and —N—;
—R is C₁₋₅₀ alkyl, which C₁₋₅₀ alkyl is optionally interrupted by one or more groups selected from the group consisting of -T-, —C(O)O—, —O—, —C(O)—, —C(O)N(R^{z1})—, —S(O)₂N(R^{z1})—, —S(O)N(R^{z1})—, —S(O)₂—, —S(O)—, —N(R^{z1})S(O)₂N(R^{z1a})—, —S—, —N(R^{z1})—, —OC(OR^{z1})(R^{z1a})—, —N(R^{z1})C(O)N(R^{z1a})—, and —OC(O)N(R^{z1})—; and which C₁₋₅₀ alkyl is optionally substituted with one or more —R^{z2};
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R⁷², which are the same or different;
each —R^{z2} is independently selected from the group consisting of halogen, —CN, oxo (═O), —COOR^{z3}, —OR^{z3}, —C(O)R^{z3}, —C(O)N(R^{z3}R^{z3a}), —S(O)₂N(R^{z3}R^{z3a}), —S(O)N(R^{z3}R^{z3a}), —S(O)₂R^{z3}, —S(O)R^{z3}, —N(R^{z3})S(O)₂N(R^{z3a}R^{z3b}), —SR^{z3}, —N(R^{z3}R^{z3a}), —NO₂, —OC(O)R^{z3}, —N(R^{z3})C(O)R^{z3a}, —N(R^{z3})S(O)₂R^{z3a}, —N(R^{z3})S(O)R^{z3a}, —N(R^{z3})C(O)O R^{z3a}, —N(R^{z3})C(O)N(R^{z3a}R^{z3b}), —OC(O)N(R^{z3}R^{z3a}), and C₁₋₆ alkyl; wherein C₁₋₆ alkyl is optionally substituted with one or more halogen, which are the same or different; and
each —R^{z1}, —R^{z1a}, —R^{z3}, —R^{z3a} and —R^{z3b} is independently selected from the group consisting of —H, and C₁₋₆ alkyl, wherein C₁₋₆ alkyl is optionally substituted with one or more halogen, which are the same or different;
wherein -L¹- is substituted with at least one -L²- and wherein -L¹- is optionally further substituted.

In certain embodiments -L¹- is substituted with one -L²-.

In one embodiment -L¹- of formula (X) is not further substituted.

In certain embodiments ═X¹ of formula (X) is selected from the group consisting of ═N and ═O. In certain embodiments ═X¹ of formula (X) is ═N. In certain embodiments ═X¹ of formula (X) is ═O.

In certain embodiments —X²— of formula (X) is selected from the group consisting of —N— and —O—. In certain embodiments —X²— of formula (X) is —N—. In certain embodiments —X²— of formula (X) is —O—.

In certain embodiments ═X¹ of formula (X) is ═N and —X²— of formula (X) is —O—. In certain embodiments ═X¹ of formula (X) is ═O and —X²— of formula (X) is —N—. In certain embodiment ═X¹ of formula (X) is ═N and —X²— of formula (X) is —N—. In certain embodiments ═X¹ of formula (X) is ═O and —X²— of formula (X) is —O—.

In certain embodiments —R of formula (X) is C₁₋₂₀ alkyl, which C₁₋₂₀ alkyl is optionally interrupted by one or more groups selected from the group consisting of -T-, —C(O)O—, —O—, —C(O)—, —C(O)N(R^{z1})—, —S(O)₂N(R^{z1})—, —S(O)N(R^{z1})—, —S(O)₂—, —S(O)—, —S—, —N(R^{z1})—, —OC(OR^{z1})(R^{z1a})—, —N(R^{z1})C(O)N(R^{z1a})—, and —OC(O)N(R^{z1})—; and which C₁₋₂₀ alkyl is optionally substituted with one or more —R^{z2};
each —R^{z1} and —R^{z1a} is independently selected from the group consisting of —H, and C₁₋₆ alkyl, wherein C₁₋₆ alkyl is optionally substituted with one or more halogen, which are the same or different;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, wherein each T is independently optionally substituted with one or more —R⁷², which are the same or different;
each —R⁷² is independently selected from the group consisting of halogen, and C₁₋₆ alkyl;
wherein C₁₋₆ alkyl is optionally substituted with one or more halogen, which are the same or different.

In certain embodiments the moiety of formula (X) is selected from the group consisting of formula (X-1), (X-2), (X-3), (X-4), (X-5), (X-6), (X-7), (X-8), (X-9), (X-10), (X-11) and (X-12)

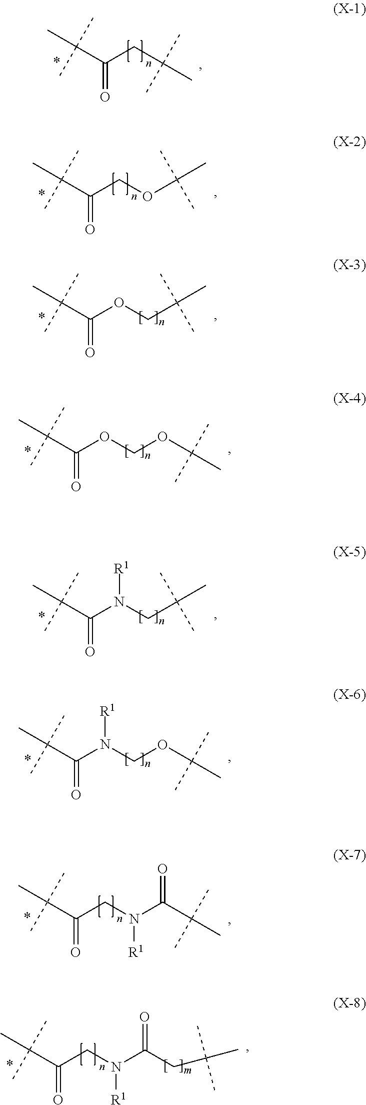

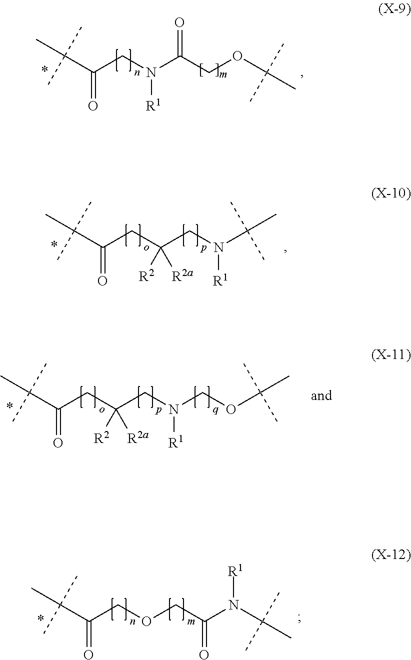

wherein
the dashed line marked with the asterisk indicates attachment to a nitrogen of an amine functional group of -D;
the unmarked dashed line indicates attachment to -L²-;
—R¹ is selected from the group consisting of —H, C₁₋₁₀ alkyl, C₂₋₁₀ alkenyl and C₂₋₁₀ alkynyl;
—R² and —R^{2a} are independently selected from the group consisting of —H, halogen, C₁₋₁₀ alkyl, C₂₋₁₀ alkenyl and C₂₋₁₀ alkynyl;
n is an integer selected from the group consisting of 1, 2, 3, 4, 5, 6, 7, 8, 9, 10, 11, 12, 13, 14, 15, 16, 17, 18, 19, 20, 21, 22, 23, 24 and 25;
m is an integer selected from the group consisting of 1, 2, 3, 4, 5, 6, 7, 8, 9, 10, 11, 12, 13, 14, 15, 16, 17, 18, 19, 20, 21, 22, 23, 24 and 25;
is an integer selected from the group consisting of 0, 1, 2, 3, 4, 5, 6, 7, 8, 9 and 10;
p is an integer selected from the group consisting of 0, 1, 2, 3, 4, 5, 6, 7, 8, 9 and 10; and
q is an integer selected from the group consisting of 1, 2, 3, 4, 5, 6, 7, 8, 9, 10, 11, 12, 13, 14, 15, 16, 17, 18, 19, 20, 21, 22, 23, 24 and 25.

In certain embodiments n of formula (X-1), (X-2), (X-3), (X-4), (X-5), (X-6), (X-7), (X-8), (X-9) or (X-12) is 1. In certain embodiments n of formula (X-1), (X-2), (X-3), (X-4), (X-5), (X-6), (X-7), (X-8), (X-9) or (X-12) is 2. In certain embodiments n of formula (X-1), (X-2), (X-3), (X-4), (X-5), (X-6), (X-7), (X-8), (X-9) or (X-12) is 3. In certain embodiments n of formula (X-1), (X-2), (X-3), (X-4), (X-5), (X-6), (X-7), (X-8), (X-9) or (X-12) is 4. In certain embodiments n of formula (X-1), (X-2), (X-3), (X-4), (X-5), (X-6), (X-7), (X-8), (X-9) or (X-12) is 5. In certain embodiments n of formula (X-1), (X-2), (X-3), (X-4), (X-5), (X-6), (X-7), (X-8), (X-9) or (X-12) is 6. In certain embodiments n of formula (X-1), (X-2), (X-3), (X-4), (X-5), (X-6), (X-7), (X-8), (X-9) or (X-12) is 7. In certain embodiments n of formula (X-1), (X-2), (X-3), (X-4), (X-5), (X-6), (X-7), (X-8), (X-9) or (X-12) is 8. In certain embodiments n of formula (X-1), (X-2), (X-3), (X-4), (X-5), (X-6), (X-7), (X-8), (X-9) or (X-12) is 9. In certain embodiments n of formula (X-1), (X-2), (X-3), (X-4), (X-5), (X-6), (X-7), (X-8), (X-9) or (X-12) is 10.

In certain embodiments m of formula (X-8), (X-9) or (X-12) is 1. In certain embodiments m of formula (X-8), (X-9) or (X-12) is 2. In certain embodiments m of formula (X-8), (X-9) or (X-12) is 3. In certain embodiments m of formula (X-8), (X-9) or (X-12) is 4. In certain embodiments m of formula (X-8), (X-9) or (X-12) is 5. In certain embodiments m of formula (X-8), (X-9) or (X-12) is 6. In certain embodiments m of formula (X-8), (X-9) or (X-12) is 7. In certain embodiments m of formula (X-8), (X-9) or (X-12) is 8. In certain embodiments m of formula (X-8), (X-9) or (X-12) is 9. In certain embodiments m of formula (X-8), (X-9) or (X-12) is 10.

In certain embodiments o of formula (X-10) or (X-11) is 0. In certain embodiments o of formula (X-10) or (X-11) is 1. In certain embodiments o of formula (X-10) or (X-11) is 2. In certain embodiments o of formula (X-10) or (X-11) is 3. In certain embodiments o of formula (X-10) or (X-11) is 4. In certain embodiments o of formula (X-10) or (X-11) is 5. In certain embodiments o of formula (X-10) or (X-11) is 6. In certain embodiments o of formula (X-10) or (X-11) is 7. In certain embodiments o of formula (X-10) or (X-11) is 8. In certain embodiments o of formula (X-10) or (X-11) is 9. In certain embodiments o of formula (X-10) or (X-11) is 10.

In certain embodiments p of formula (X-10) or (X-11) is 0. In certain embodiments p of formula (X-10) or (X-11) is 1. In certain embodiments p of formula (X-10) or (X-11) is 2. In certain embodiments p of formula (X-10) or (X-11) is 3. In certain embodiments p of formula (X-10) or (X-11) is 4. In certain embodiments p of formula (X-10) or (X-11) is 5. In certain embodiments p of formula (X-10) or (X-11) is 6. In certain embodiments p of formula (X-10) or (X-11) is 7. In certain embodiments p of formula (X-10) or (X-11) is 8. In certain embodiments p of formula (X-10) or (X-11) is 9. In certain embodiments p of formula (X-10) or (X-11) is 10.

In certain embodiments q of formula (X-11) is 1. In certain embodiments q of formula (X-11) is 2. In certain embodiments q of formula (X-11) is 3. In certain embodiments q of formula (X-11) is 4. In certain embodiments q of formula (X-11) is 5. In certain embodiments q of formula (X-11) is 6. In certain embodiments q of formula (X-11) is 7. In certain embodiments q of formula (X-11) is 8. In certain embodiments q of formula (X-11) is 9. In certain embodiments q of formula (X-11) is 10.

In certain embodiments —R¹ of formula (X-5), (X-6), (X-7), (X-8), (X-9), (X-10), (X-11) or (X-12) is —H. In certain embodiments —R¹ of formula (X-5), (X-6), (X-7), (X-8), (X-9), (X-10), (X-11) or (X-12) is C₁₋₁₀ alkyl, such as methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl or 3,3-dimethylpropyl. In certain embodiments —R¹ of formula (X-5), (X-6), (X-7), (X-8), (X-9), (X-10), (X-11) or (X-12) is C₂₋₁₀ alkenyl. In certain embodiments —R¹ of formula (X-5), (X-6), (X-7), (X-8), (X-9), (X-10), (X-11) or (X-12) is C₂₋₁₀ alkynyl.

In certain embodiments —R² of formula (X-10) or (X-11) is —H. In certain embodiments —R² of formula (X-10) or (X-11) is halogen, such as fluoro or chloro. In certain embodiments —R² of formula (X-10) or (X-11) is C₁₋₁₀ alkyl, such as methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl or 3,3-dimethylpropyl. In certain embodiments —R² of formula (X-10) or (X-11) is C₂₋₁₀ alkenyl, such as C₂ alkenyl, C₃ alkenyl, C₄ alkenyl, C₅ alkenyl or C₆ alkenyl. In certain embodiments —R² of formula (X-10) or (X-11) is C₂₋₁₀ alkynyl, such as C₂ alkynyl, C₃ alkynyl, C₄ alkynyl, C₅ alkynyl or C₆ alkynyl.

In certain embodiments —R^{2a} of formula (X-10) or (X-11) is —H. In certain embodiments —R^{2a} of formula (X-10) or (X-11) is halogen. In certain embodiments —R^{2a} of formula (X-10) or (X-11) is C₁₋₁₀ alkyl, such as methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl or 3,3-dimethylpropyl. In certain embodiments —R^{2a} of formula (X-10) or (X-11) is C₂₋₁₀ alkenyl, such as C₂ alkenyl, C₃ alkenyl, C₄ alkenyl, C₅ alkenyl or C₆ alkenyl. In certain embodiments —R^{2a} of formula (X-10) or (X-11) is C₂₋₁₀ alkynyl, such as C₂ alkynyl, C₃ alkynyl, C₄ alkynyl, C₅ alkynyl or C₆ alkynyl.

In certain embodiments at least one of —R² and —R^{2a} of formula (X-10) and (X-11) is not —H.

In certain embodiments -L¹- is of formula (X-1). In certain embodiments -L¹- is of formula (X-1) with n=1. In certain embodiments -L¹- is of formula (X-1) with n=2. In certain embodiments -L¹- is of formula (X-1) with n=3. In certain embodiments -L¹- is of formula (X-1) with n=4. In certain embodiments -L¹- is of formula (X-1) with n=5.

In certain embodiments -L¹- is of formula (X-2). In certain embodiments -L¹- is of formula (X-2) with n=1. In certain embodiments -L¹- is of formula (X-2) with n=2. In certain embodiments -L¹- is of formula (X-2) with n=3. In certain embodiments -L¹- is of formula (X-2) with n=4. In certain embodiments -L¹- is of formula (X-2) with n=5.

In certain embodiments -L¹- is of formula (X-3). In certain embodiments -L¹- is of formula (X-3) with n=1. In certain embodiments -L¹- is of formula (X-3) with n=2. In certain embodiments -L¹- is of formula (X-3) with n=3. In certain embodiments -L¹- is of formula (X-3) with n=4. In certain embodiments -L¹- is of formula (X-3) with n=5.

In certain embodiments -L¹- is of formula (X-4). In certain embodiments -L¹- is of formula (X-4) with n=1. In certain embodiments -L¹- is of formula (X-4) with n=2. In certain embodiments -L¹- is of formula (X-4) with n=3. In certain embodiments -L¹- is of formula (X-4) with n=4. In certain embodiments -L¹- is of formula (X-4) with n=5.

In certain embodiments -L¹- is of formula (X-5). In certain embodiments -L¹- is of formula (X-5) and —R¹ is —H. In certain embodiments -L¹- is of formula (X-5) and —R¹ is methyl. In certain embodiments -L¹- is of formula (X-5) and —R¹ is ethyl. In certain embodiments -L¹- is of formula (X-5) and n is 1. In certain embodiments -L¹- is of formula (X-5) and n is 2. In certain embodiments -L¹- is of formula (X-5) and n is 3. In certain embodiments -L¹- is of formula (X-5), —R¹ is —H and n is 1. In certain embodiments -L¹- is of formula (X-5), —R¹ is —H and n is 2. In certain embodiments -L¹- is of formula (X-5), —R¹ is —H and n is 3. In certain embodiments -L¹- is of formula (X-5), —R¹ is methyl and n is 1. In certain embodiments -L¹- is of formula (X-5), —R¹ is methyl and n is 2. In certain embodiments -L¹- is of formula (X-5), —R¹ is methyl and n is 3.

In certain embodiments -L¹- is of formula (X-6). In certain embodiments -L¹- is of formula (X-6) and —R¹ is —H. In certain embodiments -L¹- is of formula (X-6) and —R¹ is methyl. In certain embodiments -L¹- is of formula (X-6) and —R¹ is ethyl. In certain embodiments -L¹- is of formula (X-6) and n is 1. In certain embodiments -L¹- is of formula (X-6) and n is 2. In certain embodiments -L¹- is of formula (X-6) and n is 3. In certain embodiments -L¹- is of formula (X-6), —R¹ is —H and n is 1. In certain embodiments -L¹- is of formula (X-6), —R¹ is —H and n is 2. In certain embodiments -L¹- is of formula (X-6), —R¹ is —H and n is 3. In certain embodiments -L¹- is of formula (X-6), —R¹ is methyl and n is 1. In certain embodiments -L¹- is of formula (X-6), —R¹ is methyl and n is 2. In certain embodiments -L¹- is of formula (X-6), —R¹ is methyl and n is 3.

In certain embodiments -L¹- is of formula (X-7). In certain embodiments -L¹- is of formula (X-7) and —R¹ is —H. In certain embodiments -L¹- is of formula (X-7) and —R¹ is methyl. In certain embodiments -L¹- is of formula (X-7) and —R¹ is ethyl. In certain embodiments -L¹- is of formula (X-7) and n is 1. In certain embodiments -L¹- is of formula (X-7) and n is 2. In certain embodiments -L¹- is of formula (X-7) and n is 3. In certain embodiments -L¹- is of formula (X-7), —R¹ is —H and n is 1. In certain embodiments -L¹- is of formula (X-7), —R¹ is —H and n is 2.

In certain embodiments -L¹- is of formula (X-7), —R¹ is —H and n is 3. In certain embodiments -L¹- is of formula (X-7), —R¹ is methyl and n is 1. In certain embodiments -L¹- is of formula (X-7), —R¹ is methyl and n is 2. In certain embodiments -L¹- is of formula (X-7), —R¹ is methyl and n is 3.

In certain embodiments -L¹- is of formula (X-8). In certain embodiments -L¹- is of formula (X-8) and —R¹ is —H. In certain embodiments -L¹- is of formula (X-8) and —R¹ is methyl. In certain embodiments -L¹- is of formula (X-8) and —R¹ is ethyl. In certain embodiments -L¹- is of formula (X-8) and n is 1. In certain embodiments -L¹- is of formula (X-8) and n is 2. In certain embodiments -L¹- is of formula (X-8) and n is 3. In certain embodiments -L¹- is of formula (X-8) and m is 1. In certain embodiments -L¹- is of formula (X-8) and m is 2. In certain embodiments -L¹- is of formula (X-8) and m is 3. In certain embodiments -L¹- is of formula (X-8), —R¹ is —H, n is 1 and m is 1. In certain embodiments -L¹- is of formula (X-8), —R¹ is —H, n is 1 and m is 2. In certain embodiments -L¹- is of formula (X-8), —R¹ is —H, n is 1 and m is 3. In certain embodiments -L¹- is of formula (X-8), —R¹ is —H, n is 2 and m is 1. In certain embodiments -L¹- is of formula (X-8), —R¹ is —H, n is 2 and m is 2. In certain embodiments -L¹- is of formula (X-8), —R¹ is —H, n is 2 and m is 3. In certain embodiments -L¹- is of formula (X-8), —R¹ is —H, n is 3 and m is 1. In certain embodiments -L¹- is of formula (X-8), —R¹ is —H, n is 3 and m is 2. In certain embodiments -L¹- is of formula (X-8), —R¹ is —H, n is 3 and m is 3.

In certain embodiments -L¹- is of formula (X-9). In certain embodiments -L¹- is of formula (X-9) and —R¹ is —H. In certain embodiments -L¹- is of formula (X-9) and —R¹ is methyl. In certain embodiments -L¹- is of formula (X-9) and —R¹ is ethyl. In certain embodiments -L¹- is of formula (X-9) and n is 1. In certain embodiments -L¹- is of formula (X-9) and n is 2. In certain embodiments -L¹- is of formula (X-9) and n is 3. In certain embodiments -L¹- is of formula (X-9) and m is 1. In certain embodiments -L¹- is of formula (X-9) and m is 2. In certain embodiments -L¹- is of formula (X-9) and m is 3. In certain embodiments -L¹- is of formula (X-9), —R¹ is —H, n is 1 and m is 1. In certain embodiments -L¹- is of formula (X-9), —R¹ is —H, n is 1 and m is 2. In certain embodiments -L¹- is of formula (X-9), —R¹ is —H, n is 1 and m is 3. In certain embodiments -L¹- is of formula (X-9), —R¹ is —H, n is 2 and m is 1. In certain embodiments -L¹- is of formula (X-9), —R¹ is —H, n is 2 and m is 2. In certain embodiments -L¹- is of formula (X-9), —R¹ is —H, n is 2 and m is 3. In certain embodiments -L¹- is of formula (X-9), —R¹ is —H, n is 3 and m is 1. In certain embodiments -L¹- is of formula (X-9), —R¹ is —H, n is 3 and m is 2. In certain embodiments -L¹- is of formula (X-9), —R¹ is —H, n is 3 and m is 3.

In certain embodiments -L¹- is of formula (X-10). In certain embodiments —R¹ of formula (X-10) is —H. In certain embodiments o of formula (X-10) is 0. In certain embodiments o of formula (X-10) is 1. In certain embodiments o of formula (X-10) is 2. In certain embodiments o of formula (X-10) is 3. In certain embodiments p of formula (X-10) is 0. In certain embodiments p of formula (X-10) is 1. In certain embodiments p of formula (X-10) is 2. In certain embodiments p of formula (X-10) is 3. In certain embodiments —R² of formula (X-10) is —H. In certain embodiments —R² of formula (X-10) is halogen, such as fluor. In certain embodiments —R² of formula (X-10) is methyl. In certain embodiments —R² of formula (X-10) is ethyl. In certain embodiments —R² of formula (X-10) is n-propyl. In certain embodiments —R² of formula (X-10) is isopropyl. In certain embodiments —R² of formula (X-10) is 2-methylpropyl. In certain embodiments —R² of formula (X-10) is 2-methylpropyl. In certain embodiments —R² of formula (X-10) is 1-methylpropyl. In certain embodiments —R^{2a} of formula (X-10) is —H. In certain embodiments both —R² and —R^{2a} of formula (X-10) are methyl. In certain embodiments —R² of formula (X-10) is fluor and —R^{2a} of formula (X-10) is —H. In certain embodiments —R² of formula (X-10) is isopropyl and —R^{2a} of formula (X-10) is —H. In certain embodiments —R² of formula (X-10) is 2-methylpropyl and —R^{2a} of formula (X-10) is —H.

In certain embodiments -L¹- is of formula (X-11). In certain embodiments —R¹ of formula (X-11) is —H. In certain embodiments —R¹ of formula (X-11) is methyl. In certain embodiments —R¹ of formula (X-11) is ethyl. In certain embodiments o of formula (X-11) is 0. In certain embodiments o of formula (X-11) is 1. In certain embodiments o of formula (X-11) is 2. In certain embodiments p of formula (X-11) is 0. In certain embodiments p of formula (X-11) is 1. In certain embodiments p of formula (X-11) is 2. In certain embodiments —R² of formula (X-11) is —H. In certain embodiments —R² of formula (X-11) is halogen, such as fluor. In certain embodiments —R² of formula (X-11) is methyl. In certain embodiments —R² of formula (X-11) is ethyl. In certain embodiments —R² of formula (X-11) is n-propyl. In certain embodiments —R² of formula (X-11) is isopropyl. In certain embodiments —R² of formula (X-11) is 2-methylpropyl. In certain embodiments —R² of formula (X-11) is 2-methylpropyl. In certain embodiments —R² of formula (X-11) is 1-methylpropyl. In certain embodiments —R^{2a} of formula (X-11) is —H. In certain embodiments both —R² and —R^{2a} of formula (X-11) are methyl. In certain embodiments —R² of formula (X-11) is fluor and —R^{2a} of formula (X-11) is —H. In certain embodiments —R² of formula (X-11) is isopropyl and —R^{2a} of formula (X-11) is —H. In certain embodiments —R² of formula (X-11) is 2-methylpropyl and —R^{2a} of formula (X-11) is —H. In certain embodiments q of formula (X-11) is 1. In certain embodiments q of formula (X-11) is 2. In certain embodiments q of formula (X-11) is 3.

In certain embodiments -L¹- is of formula (X-12). In certain embodiments L¹- is of formula (X-12) and n is 1. In certain embodiment L¹- is of formula (X-12) and n is 2. In certain embodiments L¹- is of formula (X-12) and n is 3. In certain embodiments L¹- is of formula (X-12) and m is 1. In certain embodiment L¹- is of formula (X-12) and m is 2. In certain embodiments L¹- is of formula (X-12) and m is 3. In certain embodiments L¹- is of formula (X-12) and both n and m are 1. In certain embodiments L¹- is of formula (X-12) and —R¹ is —H. In certain embodiments L¹- is of formula (X-12) and —R¹ is methyl. In certain embodiments L¹- is of formula (X-12) and —R¹ is ethyl.

In certain embodiments -L¹- is selected from the group consisting of

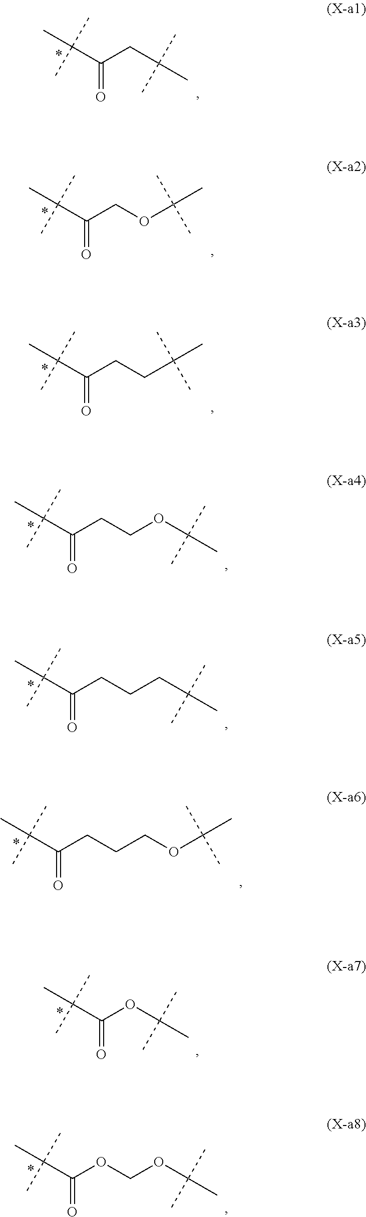

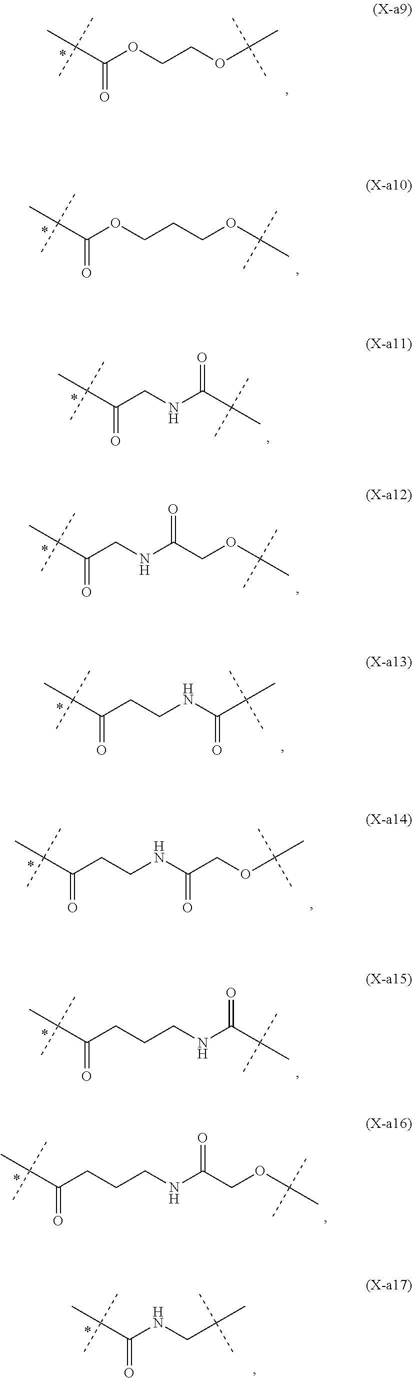

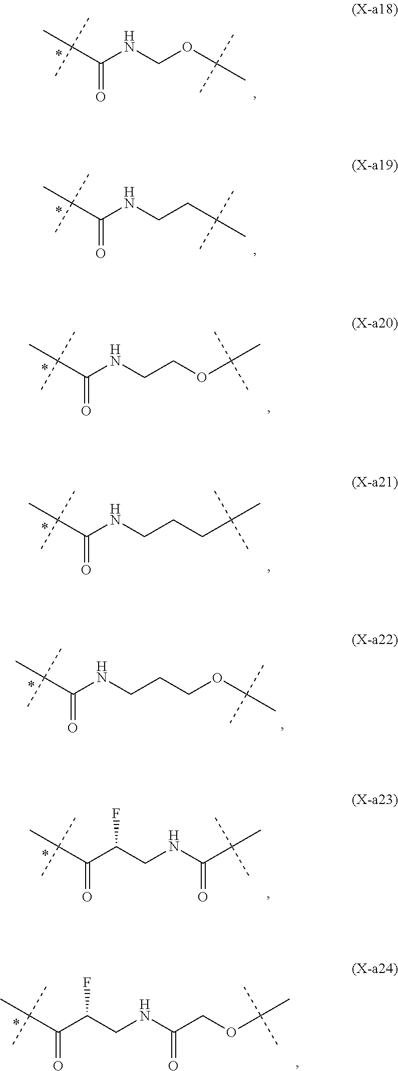

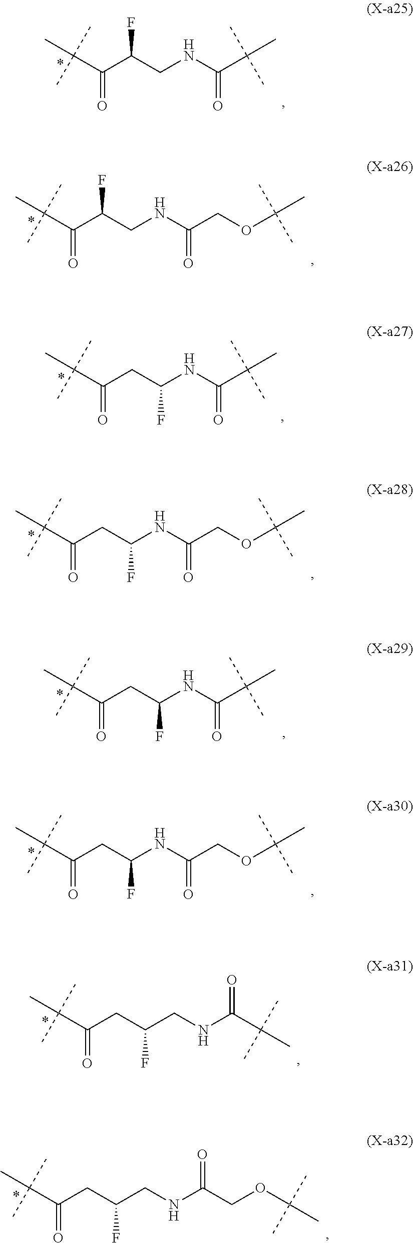

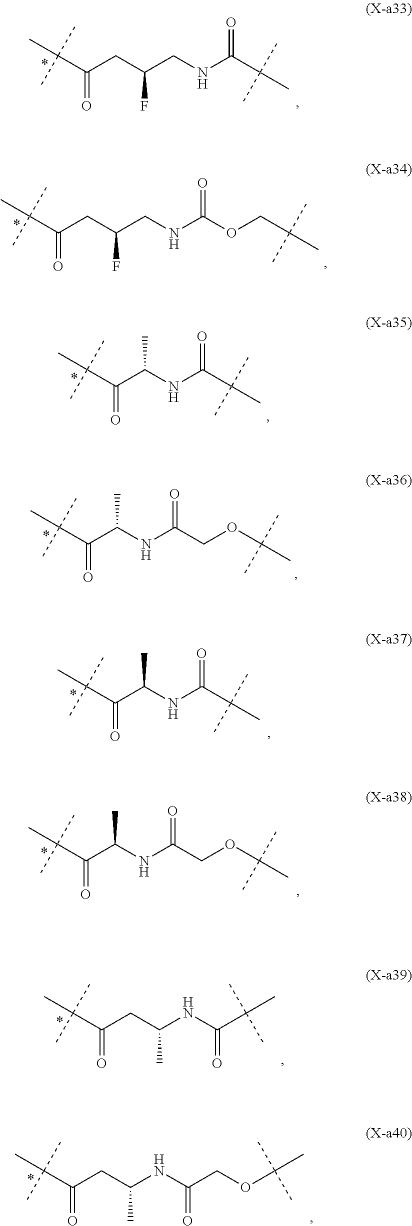

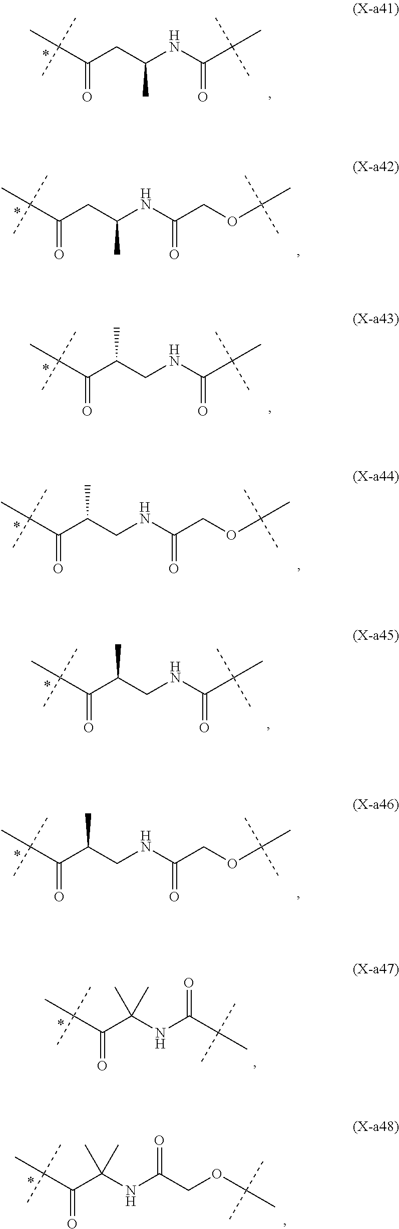

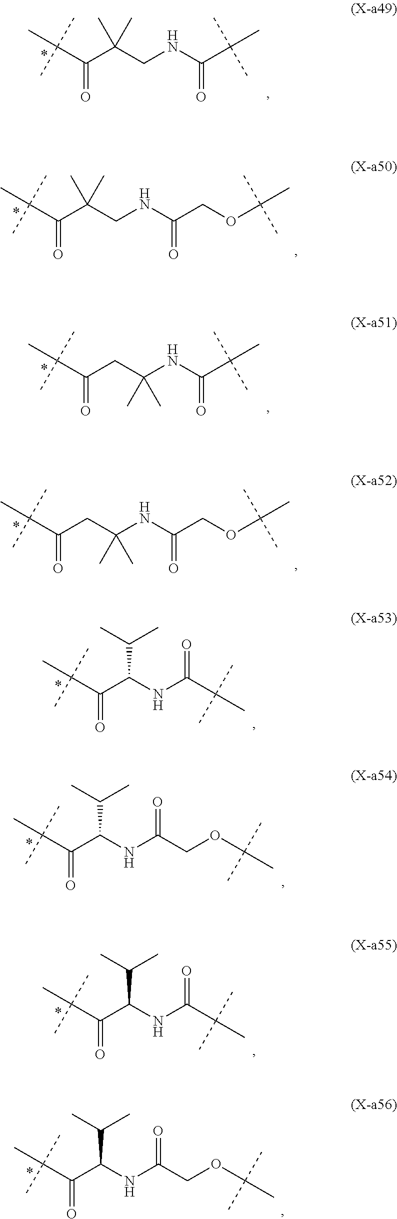

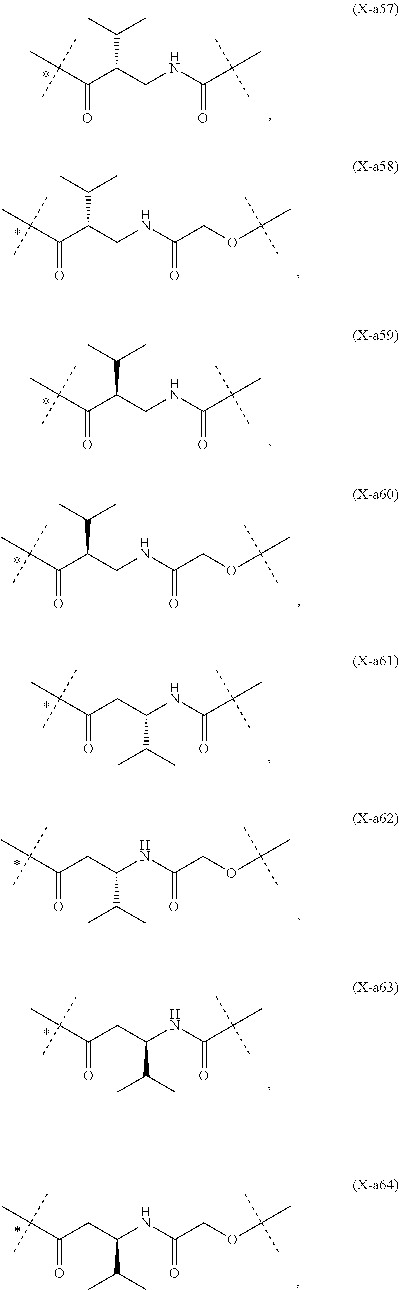

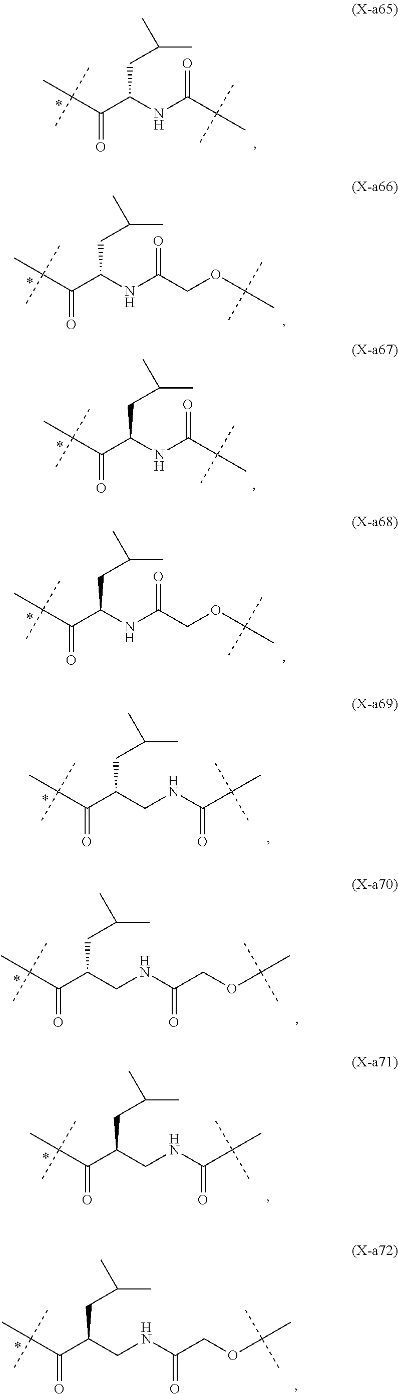

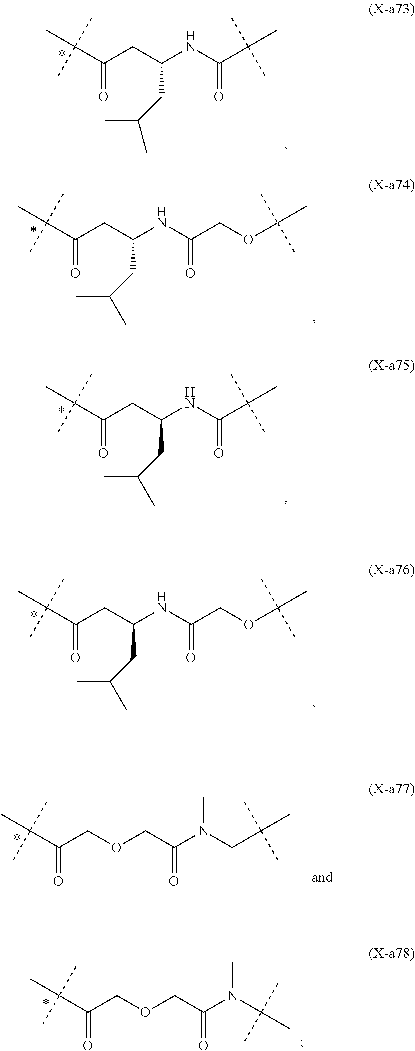

wherein
the dashed line marked with the asterisk indicates attachment to a nitrogen of an amine functional group of -D; and
the unmarked dashed line indicates attachment to -L²-.

In certain embodiments -L¹- is of formula (X-a1). In certain embodiments -L¹- is of formula (X-a2). In certain embodiments -L¹- is of formula (X-a3). In certain embodiments -L¹- is of formula (X-a4). In certain embodiments -L¹- is of formula (X-a5). In certain embodiments -L¹- is of formula (X-a6). In certain embodiments -L¹- is of formula (X-a7). In certain embodiments -L¹- is of formula (X-a8). In certain embodiments -L¹- is of formula (X-a9). In certain embodiments -L¹- is of formula (X-a10). In certain embodiments -L¹- is of formula (X-a11). In certain embodiments -L¹- is of formula (X-a12). In certain embodiments -L¹- is of formula (X-a13). In certain embodiments -L¹- is of formula (X-a14). In certain embodiments -L¹- is of formula (X-a15). In certain embodiments -L¹- is of formula (X-a16). In certain embodiments -L¹- is of formula (X-a17). In certain embodiments -L¹- is of formula (X-a18). In certain embodiments -L¹- is of formula (X-a19). In certain embodiments -L¹- is of formula (X-a20). In certain embodiments -L¹- is of formula (X-a21). In certain embodiments -L¹- is of formula (X-a22). In certain embodiments -L¹- is of formula (X-a23). In certain embodiments -L¹- is of formula (X-24). In certain embodiments -L¹- is of formula (X-a25). In certain embodiments -L¹- is of formula (X-a26). In certain embodiments -L¹- is of formula (X-a27). In certain embodiments -L¹- is of formula (X-a28). In certain embodiments -L¹- is of formula (X-a29). In certain embodiments -L¹- is of formula (X-a30). In certain embodiments -L¹- is of formula (X-a31). In certain embodiments -L¹- is of formula (X-a32). In certain embodiments -L¹- is of formula (X-a33). In certain embodiments -L¹- is of formula (X-a34). In certain embodiments -L¹- is of formula (X-a35). In certain embodiments -L¹- is of formula (X-a36). In certain embodiments -L¹- is of formula (X-a37). In certain embodiments -L¹- is of formula (X-a38). In certain embodiments -L¹- is of formula (X-a39). In certain embodiments -L¹- is of formula (X-a40). In certain embodiments -L¹- is of formula (X-a41). In certain embodiments -L¹- is of formula (X-a42). In certain embodiments -L¹- is of formula (X-a43). In certain embodiments -L¹- is of formula (X-a44). In certain embodiments -L¹- is of formula (X-a45). In certain embodiments -L¹- is of formula (X-a46). In certain embodiments -L¹- is of formula (X-a47). In certain embodiments -L¹- is of formula (X-a48). In certain embodiments -L¹- is of formula (X-a49). In certain embodiments -L¹- is of formula (X-a50). In certain embodiments -L¹- is of formula (X-a51). In certain embodiments -L¹- is of formula (X-a52). In certain embodiments -L¹- is of formula (X-a53). In certain embodiments -L¹- is of formula (X-a54). In certain embodiments -L¹- is of formula (X-a55). In certain embodiments -L¹- is of formula (X-a56). In certain embodiments -L¹- is of formula (X-a57). In certain embodiments -L¹- is of formula (X-a58). In certain embodiments -L¹- is of formula (X-a59). In certain embodiments -L¹- is of formula (X-a60). In certain embodiments -L¹- is of formula (X-a61). In certain embodiments -L¹- is of formula (X-a62). In certain embodiments -L¹- is of formula (X-a63). In certain embodiments -L¹- is of formula (X-a64). In certain embodiments -L¹- is of formula (X-a65). In certain embodiments -L¹- is of formula (X-a66). In certain embodiments -L¹- is of formula (X-a67). In certain embodiments -L¹- is of formula (X-a68). In certain embodiments -L¹- is of formula (X-a69). In certain embodiments -L¹- is of formula (X-a70). In certain embodiments -L¹- is of formula (X-a71). In certain embodiments -L¹- is of formula (X-a72). In certain embodiments -L¹- is of formula (X-a73). In certain embodiments -L¹- is of formula (X-a74). In certain embodiments -L¹- is of formula (X-a75). In certain embodiments -L¹- is of formula (X-a76). In certain embodiments -L¹- is of formula (X-a77). In certain embodiments -L¹- is of formula (X-a78).

In certain embodiments release half-life, i.e. the time in which half of all moieties-D are released from -L¹-, is pH independent, in particular independent for a pH ranging from about 6.8 to about 7.4. Such pH-independent release is advantageous, because pH in tumor tissue may vary and such pH-independence allows for a more uniform and thus more predictable drug release.

It was surprisingly found that moieties -L¹- of formula (X-a11) and (X-a12) have a release half-life that is independent of pH for a pH ranging from 6.8 to 7.4.

In certain embodiments the moiety -L¹-D is of formula (X-b1)

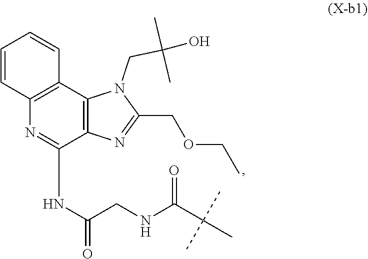

wherein the dashed line indicates attachment to -L²-.

In certain embodiments the moiety -L¹-D is of formula (X-b2)

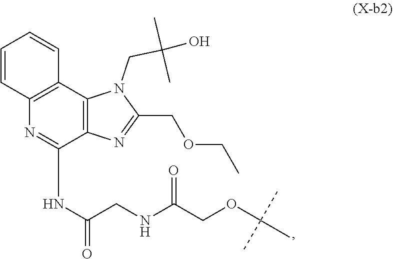

wherein the dashed line indicates attachment to -L²-.

In certain embodiments the moiety -L¹-D is of formula (X-b5)

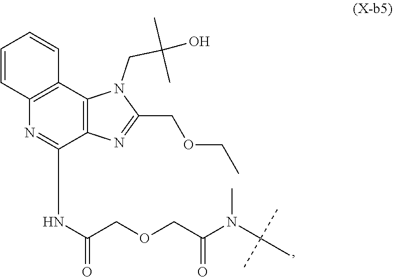

wherein the dashed line indicates attachment to -L²-.

In certain embodiments the moiety -L¹-D is of formula (X-b6)

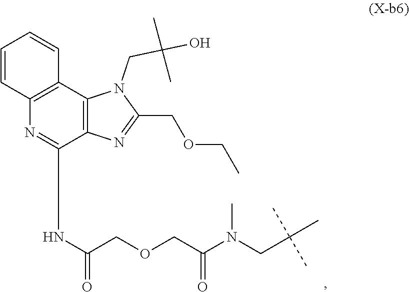

wherein the dashed line indicates attachment to -L²-.

In the conjugates of the present invention-L²- is a chemical bond or a spacer moiety. In certain embodiments -L²-does not comprise a reversible linkage, i.e. all linkages in -L²- are stable linkages. In certain embodiments -L¹- is connected to -L²-via a stable linkage. In certain embodiments -L²- is connected to —Z via a stable linkage.

In certain embodiments -L²- is a chemical bond.

In certain embodiments -L²- is a spacer moiety.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R¹³)—, —S(O)₂N(R¹³)—, —S(O)N(R^{y3})—, —S(O)₂—, —S(O)—, —N(R^{y3})S(O)₂N(R^{y3a})—, —S—, —N(R^{y3})—, —OC(OR¹³)(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -L²- is a spacer moiety selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR¹³)(R^{y3a})—, —N(R^{y3})C(O)N(R^{y3a})—, and —OC(O)N(R^{y3})—;
—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and
wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4})—, —S—, —N(R^{y4})—, —OC(OR^{y4})(R^{y4a})—, —N(R^{y4})C(O)N(R^{y4a})—, and —OC(O)N(R^{y4})—;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;
—R^{y2} is selected from the group consisting of halogen, —CN, OXO (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5}ªR^{y5b}), —SR^{y5}, —N(R^{y5}R^{y5a}), —NO₂, —OC(O)R^{y5}, —N(R^{y5}) C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;
—R^{y1} and —R^{y1a} are independently selected from the group consisting of —H, -T, C₁₋₁₀ alkyl, C₂₋₁₀ alkenyl, and C₂₋₁₀ alkynyl;
each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;
each —R^{y2} is independently selected from the group consisting of halogen, and C₁₋₆ alkyl; and
each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6}a are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight ranging from 14 g/mol to 750 g/mol.

In certain embodiments -L²-comprises a moiety selected from

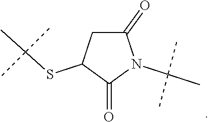

In certain embodiments -L²- has a chain lengths of 1 to 20 atoms.

As used herein the term “chain length” with regard to the moiety -L²-refers to the number of atoms of -L²-present in the shortest connection between-L¹- and —Z.

In certain embodiments -L²- is of formula (A-1)

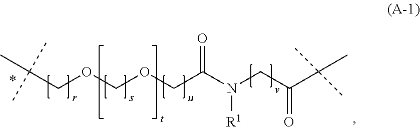

wherein
the dashed line marked with the asterisk indicates attachment to -L¹-,
the unmarked dashed line indicates attachment to Z,
r is selected from the group consisting of 1, 2, 3, 4, 5, 6, 7, 8, 9 and 10;
s is selected from the group consisting of 1, 2, 3, 4, 5, 6, 7, 8, 9 and 10;
t is selected from the group consisting of 0, 1, 2, 3, 4, 5, 6, 7, 8, 9 and 10;
u is selected from the group consisting of 1, 2, 3, 4, 5, 6, 7, 8, 9 and 10;
v is selected from the group consisting of 1, 2, 3, 4, 5, 6, 7, 8, 9 and 10; and
—R¹ is selected from the group consisting of —H, C₁₋₁₀ alkyl, C₂₋₁₀ alkenyl and C₂₋₁₀ alkynyl.

In certain embodiments r of formula (A-1) is 1. In certain embodiments r of formula (A-1) is 2. In certain embodiments r of formula (A-1) is 3. In certain embodiments r of formula (A-1) is 4. In certain embodiments r of formula (A-1) is 5. In certain embodiments r of formula (A-1) is 6. In certain embodiments r of formula (A-1) is 7. In certain embodiments r of formula (A-1) is 8. In certain embodiments r of formula (A-1) is 9. In certain embodiments r of formula (A-1) is 10.

In certain embodiments s of formula (A-1) is 1. In certain embodiments s of formula (A-1) is 2. In certain embodiments s of formula (A-1) is 3. In certain embodiments s of formula (A-1) is 4. In certain embodiments s of formula (A-1) is 5. In certain embodiments s of formula (A-1) is 6. In certain embodiments s of formula (A-1) is 7. In certain embodiments s of formula (A-1) is 8. In certain embodiments s of formula (A-1) is 9. In certain embodiments s of formula (A-1) is 10.

In certain embodiments t of formula (A-1) is 1. In certain embodiments t of formula (A-1) is 2. In certain embodiments t of formula (A-1) is 3. In certain embodiments t of formula (A-1) is 4. In certain embodiments t of formula (A-1) is 5. In certain embodiments t of formula (A-1) is 6. In certain embodiments t of formula (A-1) is 7. In certain embodiments t of formula (A-1) is 8. In certain embodiments t of formula (A-1) is 9. In certain embodiments t of formula (A-1) is 10.

In certain embodiments u of formula (A-1) is 1. In certain embodiments u of formula (A-1) is 2. In certain embodiments u of formula (A-1) is 3. In certain embodiments u of formula (A-1) is 4. In certain embodiments u of formula (A-1) is 5. In certain embodiments u of formula (A-1) is 6. In certain embodiments u of formula (A-1) is 7. In certain embodiments u of formula (A-1) is 8. In certain embodiments u of formula (A-1) is 9. In certain embodiments u of formula (A-1) is 10.

In certain embodiments v of formula (A-1) is 1. In certain embodiments v of formula (A-1) is 2. In certain embodiments v of formula (A-1) is 3. In certain embodiments v of formula (A-1) is 4. In certain embodiments v of formula (A-1) is 5. In certain embodiments v of formula (A-1) is 6. In certain embodiments v of formula (A-1) is 7. In certain embodiments v of formula (A-1) is 8. In certain embodiments v of formula (A-1) is 9. In certain embodiments v of formula (A-1) is 10.

In certain embodiments —R¹ of formula (A-1) is —H. In certain embodiments —R¹ of formula (A-1) is methyl. In certain embodiments —R¹ of formula (A-1) is ethyl. In certain embodiments —R¹ of formula (A-1) is n-propyl. In certain embodiments —R¹ of formula (A-1) is isopropyl. In certain embodiments —R¹ of formula (A-1) is n-butyl. In certain embodiments —R¹ of formula (A-1) is isobutyl. In certain embodiments —R¹ of formula (A-1) is sec-butyl. In certain embodiments —R¹ of formula (A-1) is tert-butyl. In certain embodiments —R¹ of formula (A-1) is n-pentyl. In certain embodiments —R¹ of formula (A-1) is 2-methylbutyl. In certain embodiments —R¹ of formula (A-1) is 2,2-dimethylpropyl. In certain embodiments —R¹ of formula (A-1) is n-hexyl. In certain embodiments —R¹ of formula (A-1) is 2-methylpentyl. In certain embodiments —R¹ of formula (A-1) is 3-methylpentyl. In certain embodiments —R¹ of formula (A-1) is 2,2-dimethylbutyl. In certain embodiments —R¹ of formula (A-1) is 2,3-dimethylbutyl. In certain embodiments —R¹ of formula (A-1) is 3,3-dimethylpropyl.

In certain embodiments r of formula (A-1) is 1, s of formula (A-1) is 2, t of formula (A-1) is 2, u of formula (A-1) is 1, v of formula (A-1) is 2 and —R¹ of formula (A-1) is —H.

In certain embodiments r of formula (A-1) is 1, s of formula (A-1) is 2, t of formula (A-1) is 3, u of formula (A-1) is 1, v of formula (A-1) is 2 and —R¹ of formula (A-1) is —H.

In certain embodiments r of formula (A-1) is 1, s of formula (A-1) is 2, t of formula (A-1) is 4, u of formula (A-1) is 1, v of formula (A-1) is 2 and —R¹ of formula (A-1) is —H.

In certain embodiments r of formula (A-1) is 1, s of formula (A-1) is 2, t of formula (A-1) is 5, u of formula (A-1) is 1, v of formula (A-1) is 2 and —R¹ of formula (A-1) is —H.

In certain embodiments the moiety D-L¹-L²- is of formula (A-2):

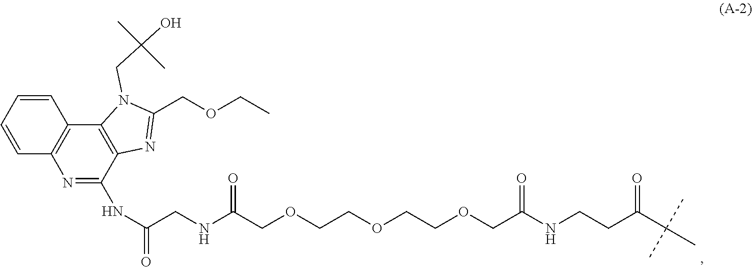

wherein
the dashed line indicates attachment to Z.

In certain embodiments Z comprises a polymer.

In certain embodiments Z is not degradable. In certain embodiments Z is degradable. A degradable moiety Z has the effect that the carrier moiety degrades over time which may be advantageous in certain applications.

In certain embodiments Z is a hydrogel. Such hydrogel may be degradable or may be non-degradable, i.e. stable. In certain embodiments such hydrogel is degradable. In certain embodiments such hydrogel is non-degradable.

In certain embodiments such hydrogel Z comprises a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(alkylene glycols), such as poly(ethylene glycols) and poly(propylene glycol), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments Z is a poly(alkylene glycol)-based hydrogel, such as a poly(propylene glycol)-based hydrogel or a poly(ethylene glycol)-based (PEG-based) hydrogel, or a hyaluronic acid-based hydrogel.

In certain embodiments Z is a PEG-based hydrogel. Such PEG-based hydrogel may be degradable or may be non-degradable, i.e. stable. In certain embodiments such PEG-based hydrogel is degradable. In certain embodiments such PEG-based hydrogel is non-degradable. Suitable hydrogels are known in the art. Examples are WO2006/003014, WO2011/012715 and WO2014/056926, which are herewith incorporated by reference.

In certain embodiments such PEG-based hydrogel comprises a plurality of backbone moieties that are crosslinked via crosslinker moieties -CL^{p}-. Optionally, there is a spacer moiety —SP¹— between a backbone moiety and a crosslinker moiety. In certain embodiments such spacer —SP¹— is defined as described above for -L²-.

In certain embodiments a backbone moiety has a molecular weight ranging from 1 kDa to 20 kDa.

In certain embodiments a backbone moiety is of formula (pA)

B*-(A-Hyp)ₓ (pA),
wherein
B* is a branching core,
A is a PEG-based polymer,
Hyp is a branched moiety,
x is an integer of from 3 to 16;
and wherein each backbone moiety is connected to one or more crosslinker moieties and to one or more moieties -L²-, which crosslinker moieties and moieties -L²- are connected to Hyp, either directly or through a spacer moiety.

In certain embodiments B* of formula (pA) is selected from the group consisting of polyalcohol moieties and polyamine moieties. In certain embodiments B* of formula (pA) is a polyalcohol moiety. In certain embodiments B* of formula (pA) is a polyamine moiety.

In certain embodiments the polyalcohol moieties for B* of formula (pA) are selected from the group consisting of a pentaerythritol moiety, tripentaerythritol moiety, hexaglycerine moiety, sucrose moiety, sorbitol moiety, fructose moiety, mannitol moiety and glucose moiety. In certain embodiments B* of formula (pA) is a pentaerythritol moiety, i.e. a moiety of formula

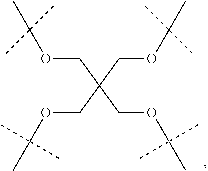

wherein dashed lines indicate attachment to -A-.

In certain embodiments the polyamine moieties for B* of formula (pA) is selected from the group consisting of an ornithine moiety, diaminobutyric acid moiety, trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety and pentadecalysine moiety. In certain embodiments B* of formula (pA) is selected from the group consisting of an ornithine moiety, diaminobutyric acid moiety and a trilysine moiety.

A backbone moiety of formula (pA) may consist of the same or different PEG-based moieties -A- and each moiety -A- may be chosen independently. In certain embodiments all moieties -A-present in a backbone moiety of formula (pA) have the same structure. It is understood that the phrase “have the same structure” with regard to polymeric moieties, such as with regard to the PEG-based polymer -A-, means that the number of monomers of the polymer, such as the number of ethylene glycol monomers, may vary due to the polydisperse nature of polymers. In certain embodiments the number of monomer units does not vary by more than a factor of 2 between all moieties -A- of a hydrogel.

In certain embodiments each -A- of formula (pA) has a molecular weight ranging from 0.3 kDa to 40 kDa; e.g. from 0.4 to 30 kDa, from 0.4 to 25 kDa, from 0.4 to 20 kDa, from 0.4 to 15 kDa, from 0.4 to 10 kDa or from 0.4 to 5 kDa. In certain embodiments each -A- has a molecular weight from 0.4 to 5 kDa. In certain embodiments -A- has a molecular weight of about 0.5 kDa. In certain embodiments -A- has a molecular weight of about 1 kDa. In certain embodiments -A- has a molecular weight of about 2 kDa. In certain embodiments -A- has a molecular weight of about 3 kDa. In certain embodiments -A- has a molecular weight of about 5 kDa.

In certain embodiments -A- of formula (pA) is of formula (pB-i)

(CH₂)ₙ₁(OCH₂CH₂)ₙX— (pB-i),
wherein
n1 is 1 or 2;
n is an integer ranging from 3 to 250, such as from 5 to 200, such as from 8 to 150 or from 10 to 100; and
X is a chemical bond or a linkage covalently linking A and Hyp.

In certain embodiments -A- of formula (pA) is of formula (pB-ii)

(CH₂)ₙ₁(OCH₂CH₂)ₙ—(CH₂)ₙ₂X— (pB-ii),
wherein
n1 is 1 or 2;
n is an integer ranging from 3 to 250, such as from 5 to 200, such as from 8 to 150 or from 10 to 100;
n2 is 0 or 1; and
X is a chemical bond or a linkage covalently linking A and Hyp.

In certain embodiments -A- of formula (pA) is of formula (pB-i′)

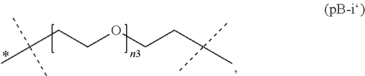

wherein
the dashed line marked with the asterisk indicates attachment to B*,
the unmarked dashed line indicates attachment to -Hyp; and
n3 is an integer ranging from 10 to 50.

In certain embodiments n3 of formula (pB-i′) is 25. In certain embodiments n3 of formula (pB-i′) is 26. In certain embodiments n3 of formula (pB-i′) is 27. In certain embodiments n3 of formula (pB-i′) is 28. In certain embodiments n3 of formula (pB-i′) is 29. In certain embodiments n3 of formula (pB-i′) is 30.

In certain embodiments a moiety B*-(A)₄ is of formula (pB-a)

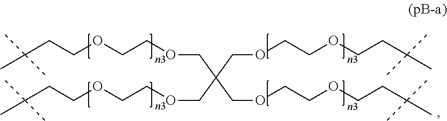

wherein
dashed lines indicate attachment to Hyp; and
each n3 is independently an integer selected from 10 to 50.

In certain embodiments n3 of formula (pB-a) is 25. In certain embodiments n3 of formula (pB-a) is 26. In certain embodiments n3 of formula (pB-a) is 27. In certain embodiments n3 of formula (B-a) is 28. In certain embodiments n3 of formula (pB-a) is 29. In certain embodiments n3 of formula (pB-a) is 30.

A backbone moiety of formula (pA) may consist of the same or different dendritic moieties-Hyp and that each -Hyp can be chosen independently. In certain embodiments all moieties-Hyp present in a backbone moiety of formula (pA) have the same structure.

In certain embodiments each -Hyp of formula (pA) has a molecular weight ranging from 0.3 kDa to 5 kDa.

In certain embodiments -Hyp is selected from the group consisting of a moiety of formula (pHyp-i)

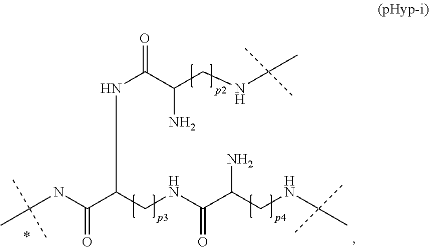

wherein
the dashed line marked with the asterisk indicates attachment to -A-,
the unmarked dashed lines indicate attachment to a spacer moiety —SP¹—, a crosslinker moiety -CL^{p}- or to -L²-; and
p2, p3 and p4 are identical or different and each is independently of the others an integer from 1 to 5;
   a moiety of formula (pHyp-ii)

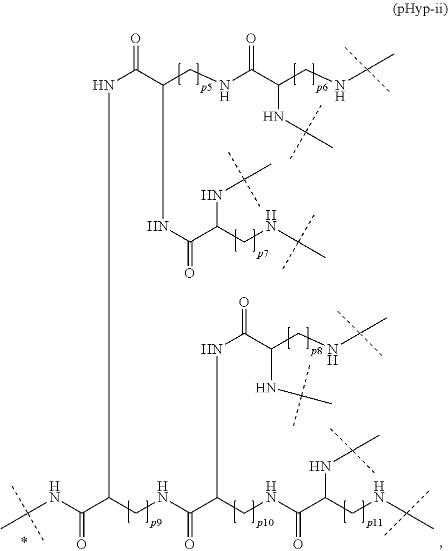

wherein
the dashed line marked with the asterisk indicates attachment to -A-,
the unmarked dashed lines indicate attachment to a spacer moiety —SP¹—, a crosslinker moiety -CL^{p}- or to -L²-; and
p5 to p11 are identical or different and each is independently of the others an integer from 1 to 5;
   a moiety of formula (pHyp-iii)

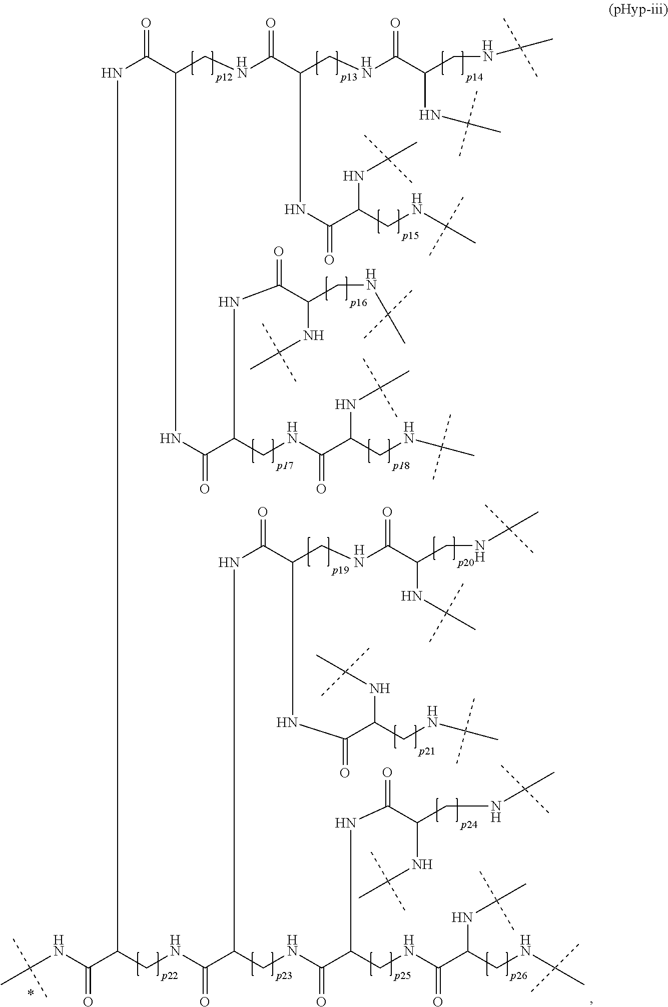

wherein
the dashed line marked with the asterisk indicates attachment to -A-,
the unmarked dashed lines indicate attachment to a spacer moiety —SP¹—, a crosslinker moiety -CL^{p}- or to -L²-; and
p12 to p26 are identical or different and each is independently of the others an integer from 1 to 5; and
   a moiety of formula (pHyp-iv)

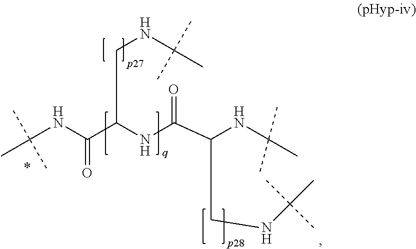

wherein
the dashed line marked with the asterisk indicates attachment to -A-,
the unmarked dashed lines indicate attachment to a spacer moiety —SP¹—, a crosslinker moiety -CL^{p}- or to -L²-;
p27 and p28 are identical or different and each is independently of the other an integer from 1 to 5; and
q is an integer from 1 to 8;
wherein the moieties (pHyp-i) to (pHyp-iv) may at each chiral center be in either R- or S-configuration.

In certain embodiments all chiral centers of a moiety (pHyp-i), (pHyp-ii), (pHyp-iii) or (pHyp-iv) are in the same configuration. In certain embodiments all chiral centers of a moiety (pHyp-i), (pHyp-ii), (pHyp-iii) or (pHyp-iv) are in R-configuration. In certain embodiments all chiral centers of a moiety (pHyp-i), (pHyp-ii), (pHyp-iii) or (pHyp-iv) are in S-configuration.

In certain embodiments p2, p3 and p4 of formula (pHyp-i) are 4.

In certain embodiments p5 to p11 of formula (pHyp-ii) are 4.

In certain embodiments p12 to p26 of formula (pHyp-iii) are 4.

In certain embodiments q of formula (pHyp-iv) is 2 or 6. In certain embodiments q of formula (pHyp-iv) q is 6.

In certain embodiments p27 and p28 of formula (pHyp-iv) are 4.

In certain embodiments -Hyp of formula (pA) comprises a branched polypeptide moiety.

In certain embodiments -Hyp of formula (pA) comprises a lysine moiety. In certain embodiments each -Hyp of formula (pA) is independently selected from the group consisting of a trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety, pentadecalysine moiety, hexadecalysine moiety, heptadecalysine moiety, octadecalysine moiety and nonadecalysine moiety.

In certain embodiments -Hyp comprises 3 lysine moieties. In certain embodiments -Hyp comprises 7 lysine moieties. In certain embodiments -Hyp comprises 15 lysine moieties. In certain embodiments -Hyp comprises heptalysinyl.

In certain embodiments x of formula (pA) is 3. In certain embodiments x of formula (pA) is 4. In certain embodiments x of formula (pA) is 6. In certain embodiments x of formula (pA) is 8.

In certain embodiments the backbone moiety is of formula (pC1)

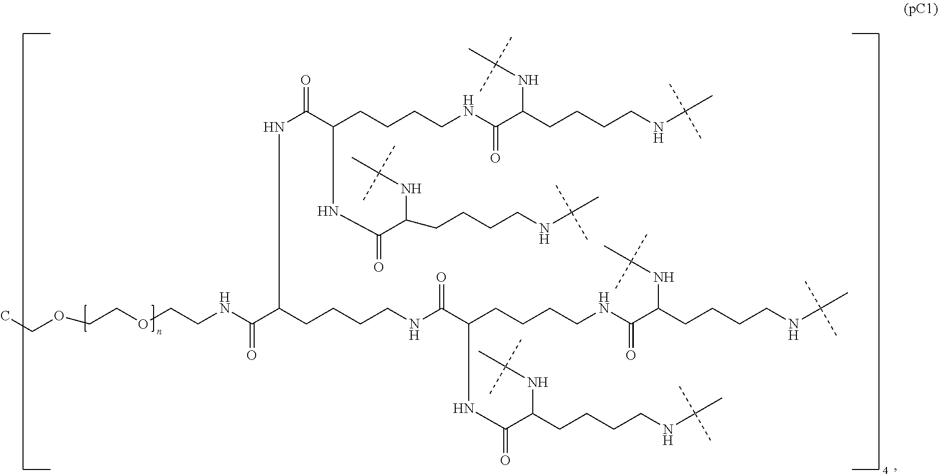

wherein
dashed lines indicate attachment to a spacer moiety —SP¹—, a crosslinker moiety -CL^{p}- or to -L²-; and
n ranges from 10 to 40.

In certain embodiments n of formula (pC1) is about 28.

In certain embodiments the backbone moiety is of formula (pC2)

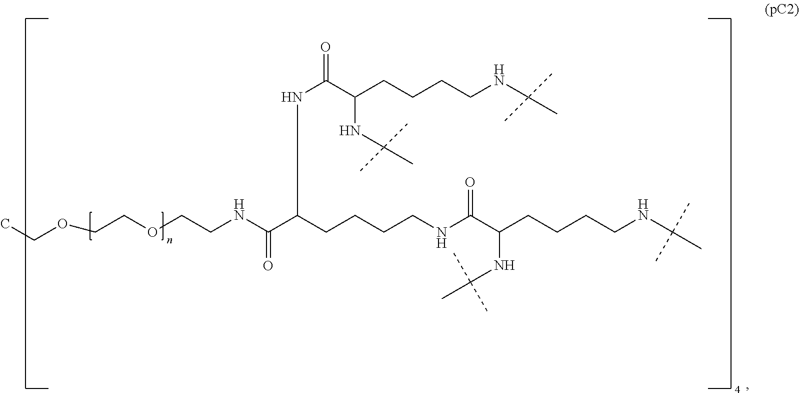

wherein
dashed lines indicate attachment to a spacer moiety —SP¹—, a crosslinker moiety -CL^{p}- or to -L²-; and
n ranges from 10 to 40.

In certain embodiments there is no spacer moiety —SP¹— between a backbone moiety and a crosslinker moiety -CL^{p}-, i.e. -CL^{p}- is directly linked to -Hyp.

The crosslinker -CL^{p}- of the PEG-based hydrogel is in certain embodiments poly(alkylene glycol) (PAG)-based. In certain embodiments the crosslinker is poly(propylene glycol)-based. In certain embodiments the crosslinker -CL^{p}- is PEG-based.

In certain embodiments such PAG-based crosslinker moiety -CL^{p}- is of formula (pD)

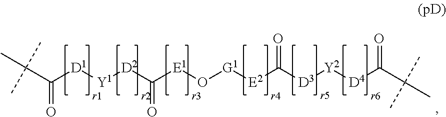

wherein
dashed lines indicate attachment to a backbone moiety or to a spacer moiety —SP¹—;
—Y¹— is of formula

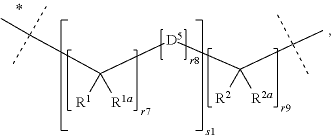

wherein the dashed line marked with the asterisk indicates attachment to -D¹- and the unmarked dashed line indicates attachment to -D²-;
—Y²— is of formula

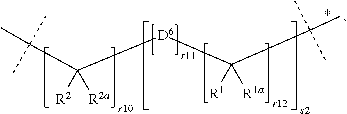

wherein the dashed line marked with the asterisk indicates attachment to -D⁴- and the unmarked dashed line indicates attachment to -D³-;
-E¹- is of formula

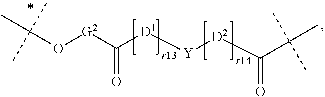

wherein the dashed line marked with the asterisk indicates attachment to —(C═O)— and the unmarked dashed line indicates attachment to —O—;
-E²- is of formula

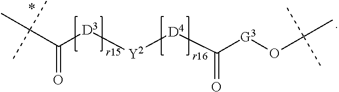

wherein the dashed line marked with the asterisk indicates attachment to -G¹- and the unmarked dashed line indicates attachment to —(C═O)—;
-G¹- is of formula

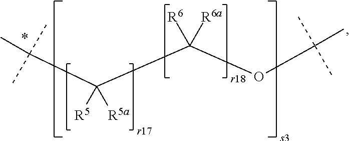

wherein the dashed line marked with the asterisk indicates attachment to —O— and the unmarked dashed line indicates attachment to -E²-;
-G²- is of formula

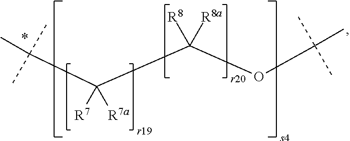

wherein the dashed line marked with the asterisk indicates attachment to —O— and the unmarked dashed line indicates attachment to —(C═O)—;
-G³- is of formula

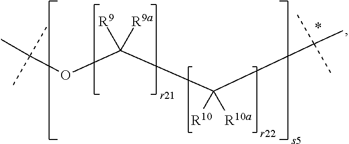

wherein the dashed line marked with the asterisk indicates attachment to —O— and the unmarked dashed line indicates attachment to —(C═O)—;
-D¹-, -D²-, -D³-, -D⁴-, -D⁵- and -D⁶- are identical or different and each is independently of the others selected from the group comprising —O—, —NR¹¹—, —N⁺R¹²R^{12a}—, —S—, —(S═O)—, —(S(O)₂)—, —C(O)—, —P(O)R¹³—, —P(O)(OR¹³) and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹, —R^{9a}, —R¹⁰, —R^{10a}, —R¹¹, —R¹², —R^{12a}, —R¹³, —R¹⁴ and —R^{14a} are identical or different and each is independently of the others selected from the group consisting of —H and C₁₋₆ alkyl;
optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R¹²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
r1, r2, r5, r6, r13, r14, r15 and r16 are independently 0 or 1;
r3, 14, 17, 18, 19, r10, r11, r12 are independently 0, 1, 2, 3, or 4;
r17, r18, r19, r20, r21 and r22 are independently 1, 2, 3, 4, 5, 6, 7, 8, 9 or 10;
s1, s2, s4, s5 are independently 1, 2, 3, 4, 5 or 6; and
s3 ranges from 1 to 900.

In certain embodiments s3 ranges from 1 to 500. In certain embodiments s3 ranges from 1 to 200.

In certain embodiments r1 of formula (pD) is 0. In certain embodiments r1 of formula (pD) is 1. In certain embodiments r2 of formula (pD) is 0. In certain embodiments r2 of formula (pD) is 1. In certain embodiments r5 of formula (pD) is 0. In certain embodiments r5 of formula (pD) is 1.

In certain embodiments r1, r2, r5 and r6 of formula (pD) are 0.

In certain embodiments r6 of formula (pD) is 0. In certain embodiments r6 of formula (pD) is 1. In certain embodiments r13 of formula (pD) is 0. In certain embodiments r13 of formula (pD) is 1. In certain embodiments r14 of formula (pD) is 0. In certain embodiments r14 of formula (pD) is 1. In certain embodiments r15 of formula (pD) is 0. In certain embodiments r15 of formula (pD) is 1. In certain embodiments r16 of formula (pD) is 0. In certain embodiments r16 of formula (pD) is 1.

In certain embodiments r3 of formula (pD) is 1. In certain embodiments r3 of formula (pD) is 2. In certain embodiments r4 of formula (pD) is 1. In certain embodiments r4 of formula (pD) is 2. In certain embodiments r3 and r4 of formula (pD) are both 1. In certain embodiments r3 and r4 of formula (pD) are both 2. In certain embodiments r3 and r4 of formula (pD) are both 3.

In certain embodiments r7 of formula (pD) is 0. In certain embodiments r7 of formula (pD) is 1. In certain embodiments r7 of formula (pD) is 2. In certain embodiments r8 of formula (pD) is 0. In certain embodiments r8 of formula (pD) is 1. In certain embodiments r8 of formula (pD) is 2. In certain embodiments r9 of formula (pD) is 0. In certain embodiments r9 of formula (pD) is 1. In certain embodiments r9 of formula (pD) is 2. In certain embodiments r10 of formula (pD) is 0. In certain embodiments r10 of formula (pD) is 1. In certain embodiments r10 of formula (pD) is 2. In certain embodiments r11 of formula (pD) is 0. In certain embodiments r11 of formula (pD) is 1. In certain embodiments r11 of formula (pD) is 2. In certain embodiments r12 of formula (pD) is 0. In certain embodiments r12 of formula (pD) is 1. In certain embodiments r12 of formula (pD) is 2.

In certain embodiments r17 of formula (pD) is 1. In certain embodiments r18 of formula (pD) is 1. In certain embodiments r19 of formula (pD) is 1. In certain embodiments r20 of formula (pD) is 1. In certain embodiments r21 of formula (pD) is 1.

In certain embodiments s1 of formula (pD) is 1. In certain embodiments s1 of formula (pD) is 2. In certain embodiments s2 of formula (pD) is 1. In certain embodiments s2 of formula (pD) is 2. In certain embodiments s4 of formula (pD) is 1. In certain embodiments s4 of formula (pD) is 2.

In certain embodiments s3 of formula (pD) ranges from 5 to 500. In certain embodiments s3 of formula (pD) ranges from 10 to 250. In certain embodiments s3 of formula (pD) ranges from 12 to 150. In certain embodiments s3 of formula (pD) ranges from 15 to 100. In certain embodiments s3 of formula (pD) ranges from 18 to 75. In certain embodiments s3 of formula (pD) ranges from 20 to 50.

In certain embodiments —R¹ of formula (pD) is —H. In certain embodiments —R¹ of formula (pD) is methyl. In certain embodiments —R¹ of formula (pD) is ethyl. In certain embodiments —R^{1a} of formula (pD) is —H. In certain embodiments —R^{1a} of formula (pD) is methyl. In certain embodiments —R^{1a} of formula (pD) is ethyl. In certain embodiments —R² of formula (pD) is —H. In certain embodiments —R² of formula (pD) is methyl. In certain embodiments —R² of formula (pD) is ethyl. In certain embodiments —R^{2a} of formula (pD) is —H. In certain embodiments —R^{2a} of formula (pD) is methyl. In certain embodiments —R^{2a} of formula (pD) is ethyl. In certain embodiments —R³ of formula (pD) is —H. In certain embodiments —R³ of formula (pD) is methyl. In certain embodiments —R³ of formula (pD) is ethyl. In certain embodiments —R^{3a} of formula (pD) is —H. In certain embodiments —R^{3a} of formula (pD) is methyl. In certain embodiments —R^{3a} of formula (pD) is ethyl. In certain embodiments —R⁴ of formula (pD) is —H. In certain embodiments —R⁴ of formula (pD) is methyl. In certain embodiments —R⁴ of formula (pD) is methyl. In certain embodiments —R^{4a} of formula (pD) is —H. In certain embodiments —R^{4a} of formula (pD) is methyl. In certain embodiments —R^{4a} of formula (pD) is ethyl. In certain embodiments —R⁵ of formula (pD) is —H. In certain embodiments —R⁵ of formula (pD) is methyl. In certain embodiments —R⁵ of formula (pD) is ethyl. In certain embodiments —R^{5a} of formula (pD) is —H. In certain embodiments —R^{5a} of formula (pD) is methyl. In certain embodiments —R^{5a} of formula (pD) is ethyl. In certain embodiments —R⁶ of formula (pD) is —H. In certain embodiments —R⁶ of formula (pD) is methyl. In certain embodiments —R⁶ of formula (pD) is ethyl. In certain embodiments —R^{6a} of formula (pD) is —H. In certain embodiments —R^{6a} of formula (pD) is methyl. In certain embodiments —R^{6a} of formula (pD) is ethyl. In certain embodiments —R⁷ of formula (pD) is —H. In certain embodiments —R⁷ of formula (pD) is methyl. In certain embodiments —R⁷ of formula (pD) is ethyl. In certain embodiments —R⁸ of formula (pD) is —H. In certain embodiments —R⁸ of formula (pD) is methyl. In certain embodiments —R⁸ of formula (pD) is ethyl. In certain embodiments —R^{8a} of formula (pD) is —H. In certain embodiments —R^{8a} of formula (pD) is methyl. In certain embodiments —R^{8a} of formula (pD) is ethyl. In certain embodiments —R⁹ of formula (pD) is —H. In certain embodiments —R⁸ of formula (pD) is methyl. In certain embodiments —R⁹ of formula (pD) is ethyl. In certain embodiments —R^{9a} of formula (pD) is —H. In certain embodiments —R^{9a} of formula (pD) is methyl. In certain embodiments —R^{9a} of formula (pD) is ethyl. In certain embodiments —R^{9a} of formula (pD) is —H. In certain embodiments —R^{9a} of formula (pD) is methyl. In certain embodiments —R^{9a} of formula (pD) is ethyl. In certain embodiments —R¹⁰ of formula (pD) is —H. In certain embodiments —R¹⁰ of formula (pD) is methyl. In certain embodiments —R¹⁰ of formula (pD) is ethyl. In certain embodiments —R^{10a} of formula (pD) is —H. In certain embodiments —R^{10a} of formula (pD) is methyl. In certain embodiments —R^{10a} of formula (pD) is ethyl. In certain embodiments —R¹¹ of formula (pD) is —H. In certain embodiments —R¹¹ of formula (pD) is methyl. In certain embodiments —R¹¹ of formula (pD) is ethyl. In certain embodiments —R¹² of formula (pD) is —H. In certain embodiments —R¹² of formula (pD) is methyl. In certain embodiments —R¹² of formula (pD) is ethyl. In certain embodiments —R^{12a} of formula (pD) is —H. In certain embodiments —R^{12a} of formula (pD) is methyl. In certain embodiments —R^{12a} of formula (pD) is ethyl. In certain embodiments —R¹³ of formula (pD) is —H. In certain embodiments —R¹³ of formula (pD) is methyl. In certain embodiments —R¹³ of formula (pD) is ethyl. In certain embodiments —R¹⁴ of formula (pD) is —H. In certain embodiments —R¹⁴ of formula (pD) is methyl. In certain embodiments —R¹⁴ of formula (pD) is ethyl. In certain embodiments —R^{14a} of formula (pD) is —H. In certain embodiments —R^{14a} of formula (pD) is methyl. In certain embodiments —R^{14a} of formula (pD) is ethyl.

In certain embodiments -D¹- of formula (pD) is —O—. In certain embodiments -D¹- of formula (pD) is —NR¹¹—. In certain embodiments -D¹- of formula (pD) is —N⁺R¹²R^{12a}. In certain embodiments -D¹- of formula (pD) is —S—. In certain embodiments -D¹- of formula (pD) is —(S═O). In certain embodiments -D¹- of formula (pD) is —(S(O)₂)—. In certain embodiments -D¹- of formula (pD) is —C(O)—. In certain embodiments -D¹- of formula (pD) is —P(O)R¹³—. In certain embodiments -D¹- of formula (pD) is —P(O)(OR¹³)—. In certain embodiments -D¹- of formula (pD) is —CR¹⁴R^{14a}.

In certain embodiments -D²- of formula (pD) is —O—. In certain embodiments -D²- of formula (pD) is —NR¹¹—. In certain embodiments -D²- of formula (pD) is —N⁺R¹²R^{12a}—. In certain embodiments -D²- of formula (pD) is —S—. In certain embodiments -D²- of formula (pD) is —(S═O). In certain embodiments -D²- of formula (pD) is —(S(O)₂)—. In certain embodiments -D²- of formula (pD) is —C(O)—. In certain embodiments -D²- of formula (pD) is —P(O)R¹³—In certain embodiments -D²- of formula (pD) is —P(O)(OR¹³)—. In certain embodiments -D²- of formula (pD) is —CR¹⁴R^{14a}.

In certain embodiments -D³- of formula (pD) is —O—. In certain embodiments -D³- of formula (pD) is —NR¹¹—. In certain embodiments -D³- of formula (pD) is-N′R¹²R^{12a}—. In certain embodiments -D³- of formula (pD) is —S—. In certain embodiments -D³- of formula (pD) is —(S═O). In certain embodiments -D³- of formula (pD) is —(S(O)₂)—. In certain embodiments -D³- of formula (pD) is —C(O)—. In certain embodiments -D³- of formula (pD) is —P(O)R¹³—. In certain embodiments -D³- of formula (PD) is —P(O)(OR¹³)—. In certain embodiments -D³- of formula (pD) is —CR¹⁴R^{14a}.

In certain embodiments -D⁴- of formula (pD) is —O—. In certain embodiments -D⁴- of formula (pD) is —NR¹¹—. In certain embodiments -D⁴- of formula (pD) is —N⁺R¹²R^{12a}—. In certain embodiments -D⁴- of formula (pD) is —S—. In certain embodiments -D⁴- of formula (pD) is —(S═O). In certain embodiments -D⁴- of formula (pD) is —(S(O)₂)—. In certain embodiments -D⁴- of formula (pD) is —C(O)—. In certain embodiments -D⁴- of formula (pD) is —P(O)R¹³—. In certain embodiments -D⁴- of formula (pD) is —P(O)(OR¹³)—. In certain embodiments -D⁴- of formula (pD) is —CR¹⁴R^{14a}.

In certain embodiments -D⁵- of formula (pD) is —O—. In certain embodiments -D⁵- of formula (pD) is —NR¹¹—. In certain embodiments -D⁵- of formula (PD) is —N⁺R¹²R^{12a}—. In certain embodiments -D⁵- of formula (pD) is —S—. In certain embodiments -D⁵- of formula (pD) is —(S═O)—. In certain embodiments -D⁵- of formula (pD) is —(S(O)₂)—. In certain embodiments -D⁵- of formula (pD) is —C(O)—. In certain embodiments -D⁵- of formula (pD) is —P(O)R¹³—In certain embodiments -D⁵- of formula (pD) is —P(O)(OR¹³)—. In certain embodiments -D⁵- of formula (pD) is —CR¹⁴R^{14a}.

In certain embodiments -D⁶- of formula (pD) is —O—. In certain embodiments -D⁶- of formula (pD) is —NR¹¹—. In certain embodiments -D⁶- of formula (pD) is —N⁺R¹²R^{12a}—. In certain embodiments -D⁶- of formula (pD) is —S—. In certain embodiments -D⁶- of formula (pD) is —(S═O). In certain embodiments -D⁶- of formula (pD) is —(S(O)₂)—. In certain embodiments -D⁶- of formula (pD) is —C(O)—. In certain embodiments -D⁶- of formula (pD) is —P(O)R¹³—In certain embodiments -D⁶- of formula (pD) is —P(O)(OR¹³)—. In certain embodiments -D⁶- of formula (pD) is —CR¹⁴R^{14a}.

In one embodiment -CL^{p}- is of formula (pE)

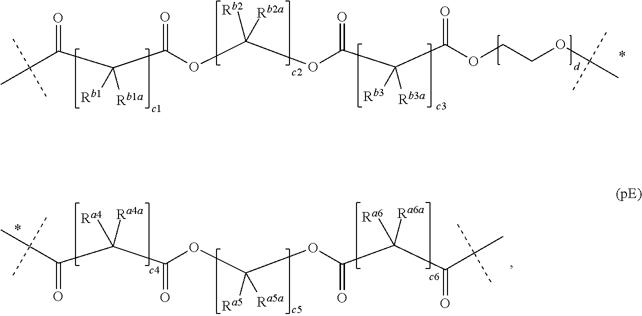

wherein
dashed lines marked with an asterisk indicate the connection point between the upper and the lower substructure,
unmarked dashed lines indicate attachment to a backbone moiety or to a spacer moiety —SP¹—;
—R^{b1}, —R^{b1a}, —R^{b2}, —R^{b2a}, —R^{b3}, —R^{b3a}, —R^{b4}, —R^{b4a}, —R^{b5}, —R^{b5a}, —R^{b6} and —R^{b6} are independently selected from the group consisting of —H and C₁₋₆ alkyl;
c1, c2, c3, c4, c5 and c6 are independently selected from the group consisting of 1, 2, 3, 4, 5 and 6;
d is an integer ranging from 2 to 250.

In certain embodiments d of formula (pE) ranges from 3 to 200. In certain embodiments d of formula (pE) ranges from 4 to 150. In certain embodiments d of formula (pE) ranges from 5 to 100. In certain embodiments d of formula (pE) ranges from 10 to 50. In certain embodiments d of formula (pE) ranges from 15 to 30. In certain embodiments d of formula (pE) is about 23.

In certain embodiments —R^{b1} and —R^{b1a} of formula (pE) are —H. In certain embodiments —R^{b1} and —R^{b1a} of formula (pE) are —H. In certain embodiments —R^{b2} and —R^{b2}a of formula (pE) are —H.

In certain embodiments —R^{b3} and —R^{b3a} of formula (pE) are —H. In certain embodiments —R^{b4} and —R^{b4a} of formula (pE) are —H. In certain embodiments —R^{b5} and —R^{b5a} of formula (pE) are —H. In certain embodiments —R^{b6} and —R^{b6}a of formula (pE) are —H.

In certain embodiments —R^{b1}, —R^{b1a}, —R^{b2}, —R^{b2a}, —R^{b3}, —R^{b3a}, —R^{b4}, —R^{b4a}, —R^{b5}, —R^{b5a}, —R^{b6} and —R^{b6} of formula (pE) are all —H.

In certain embodiments c1 of formula (pE) is 1. In certain embodiments c1 of formula (pE) is 2. In certain embodiments c1 of formula (pE) is 3. In certain embodiments c1 of formula (pE) is 4. In certain embodiments c1 of formula (pE) is 5. In certain embodiments c1 of formula (pE) is 6.

In certain embodiments c2 of formula (pE) is 1. In certain embodiments c2 of formula (pE) is 2. In certain embodiments c2 of formula (pE) is 3. In certain embodiments c2 of formula (pE) is 4. In certain embodiments c2 of formula (pE) is 5. In certain embodiments c2 of formula (pE) is 6.

In certain embodiments c3 of formula (pE) is 1. In certain embodiments c3 of formula (pE) is 2. In certain embodiments c3 of formula (pE) is 3. In certain embodiments c3 of formula (pE) is 4. In certain embodiments c3 of formula (pE) is 5. In certain embodiments c3 of formula (pE) is 6.

In certain embodiments c4 of formula (pE) is 1. In certain embodiments c4 of formula (pE) is 2. In certain embodiments c4 of formula (pE) is 3. In certain embodiments c4 of formula (pE) is 4. In certain embodiments c4 of formula (pE) is 5. In certain embodiments c4 of formula (pE) is 6.

In certain embodiments c5 of formula (pE) is 1. In certain embodiments c5 of formula (pE) is 2. In certain embodiments c5 of formula (pE) is 3. In certain embodiments c5 of formula (pE) is 4. In certain embodiments c5 of formula (pE) is 5. In certain embodiments c5 of formula (pE) is 6.

In certain embodiments c6 of formula (pE) is 1. In certain embodiments c6 of formula (pE) is 2. In certain embodiments c6 of formula (pE) is 3. In certain embodiments c6 of formula (pE) is 4. In certain embodiments c6 of formula (pE) is 5. In certain embodiments c6 of formula (pE) is 6.

In certain embodiments a crosslinker moiety -CL^{p}- is of formula (pE-i)

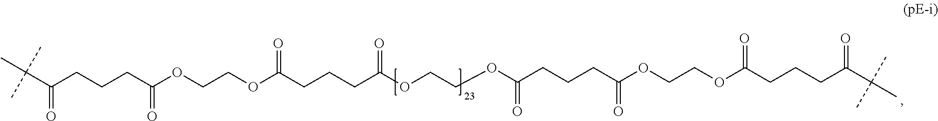

wherein
dashed lines indicate attachment to a backbone moiety or to a spacer moiety —SP¹—.

In certain embodiments —Z is a hyaluronic acid-based hydrogel. Such hyaluronic acid-based hydrogels are known in the art, such as for example from WO2018/175788, which is incorporated herewith by reference.

In certain embodiments the TLR7/8 agonist conjugate is of formula (A-3)

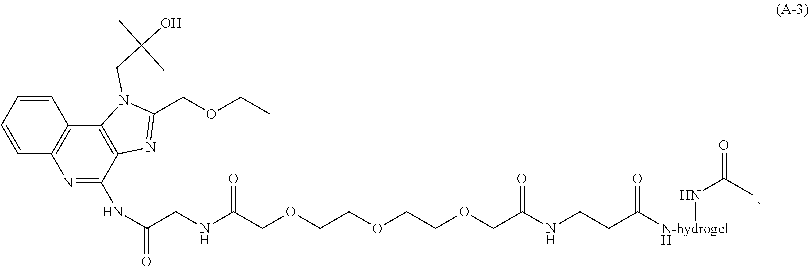

wherein “hydrogel” is a hydrogel comprising backbone moieties of formula (A-4):

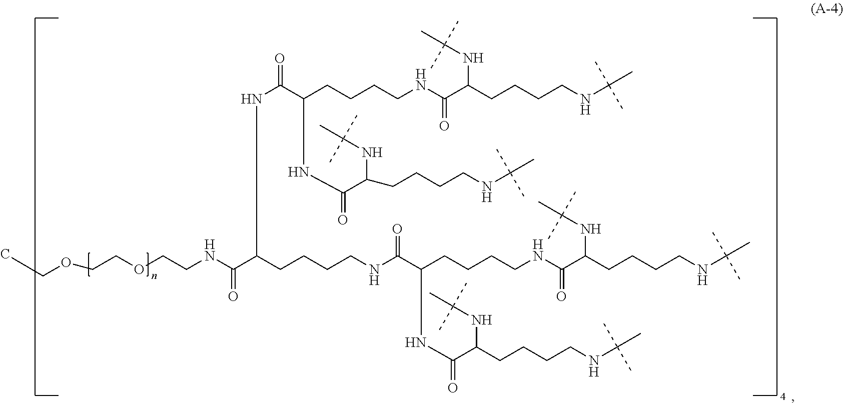

wherein n ranges from approx. 25 to 29 and dashed lines indicate attachment to either a moiety

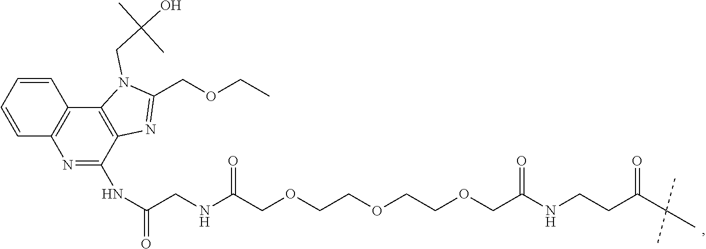

to a moiety

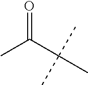

or to a crosslinker of formula (A-5):

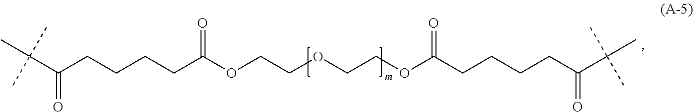

wherein m ranges from approx. 41 to 45 and the dashed lines in (A-5) indicate attachment to a backbone moiety of formula (A-4).

In certain embodiments n of formula (A-4) is approx. 28. In certain embodiments n of formula (A-5) is approx. 27.

In certain embodiments m of formula (A-5) is approx. 44. In certain embodiments m of formula (A-5) is approx. 45.

In certain embodiments n of formula (A-4) is approx. 28 and m of formula (A-5) is approx. 45.

In certain embodiments the TLR7/8 agonist conjugate is of formula (Ai-6):

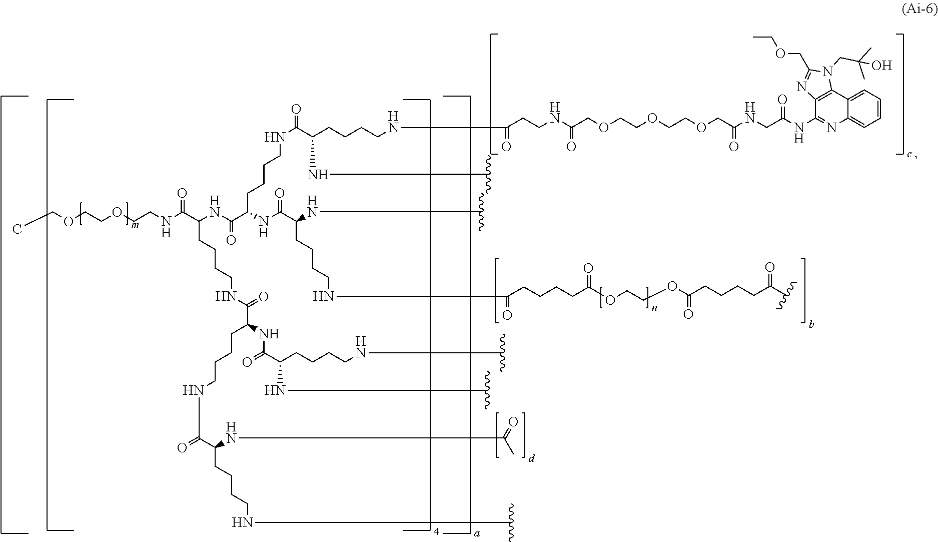

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:c:d with c+d=6.

In certain embodiments the TLR7/8 agonist conjugate is of formula (A-6):

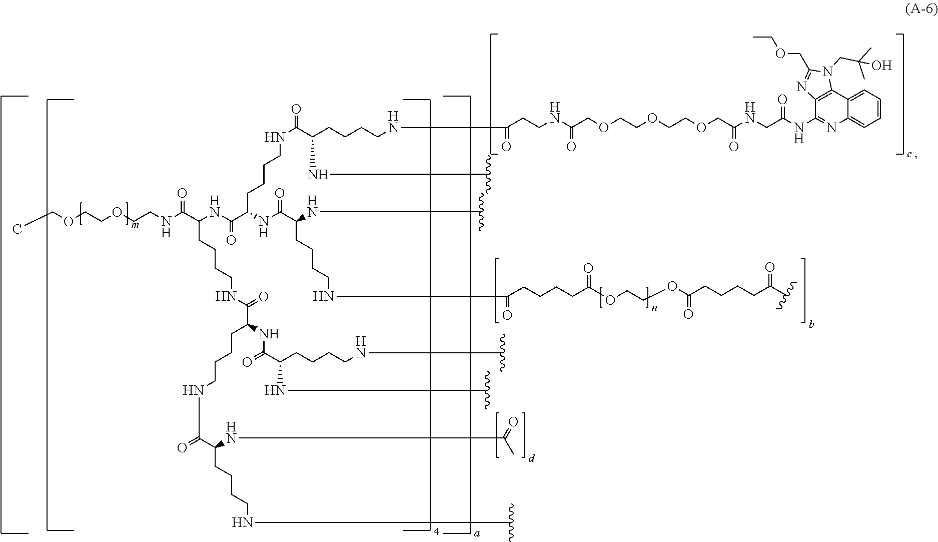

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:2:4.

It is understood that wavy lines indicate attachment to further moieties “b”, “c” or “d”, which for simplification were not shown.

In certain embodiments the ratio of “a”:“b”:“c”:“d” in (A-6) is approx. 1:13:2.4:3.6.

In certain embodiments the TLR7/8 agonist conjugate is of formula (A-7):

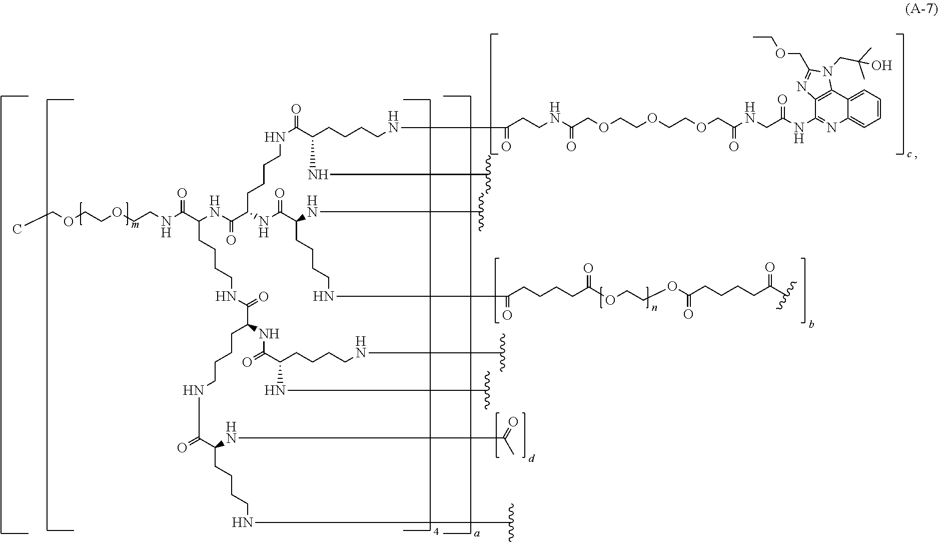

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:4:2.

In certain embodiments the TLR7/8 agonist conjugate is of formula (A-8):

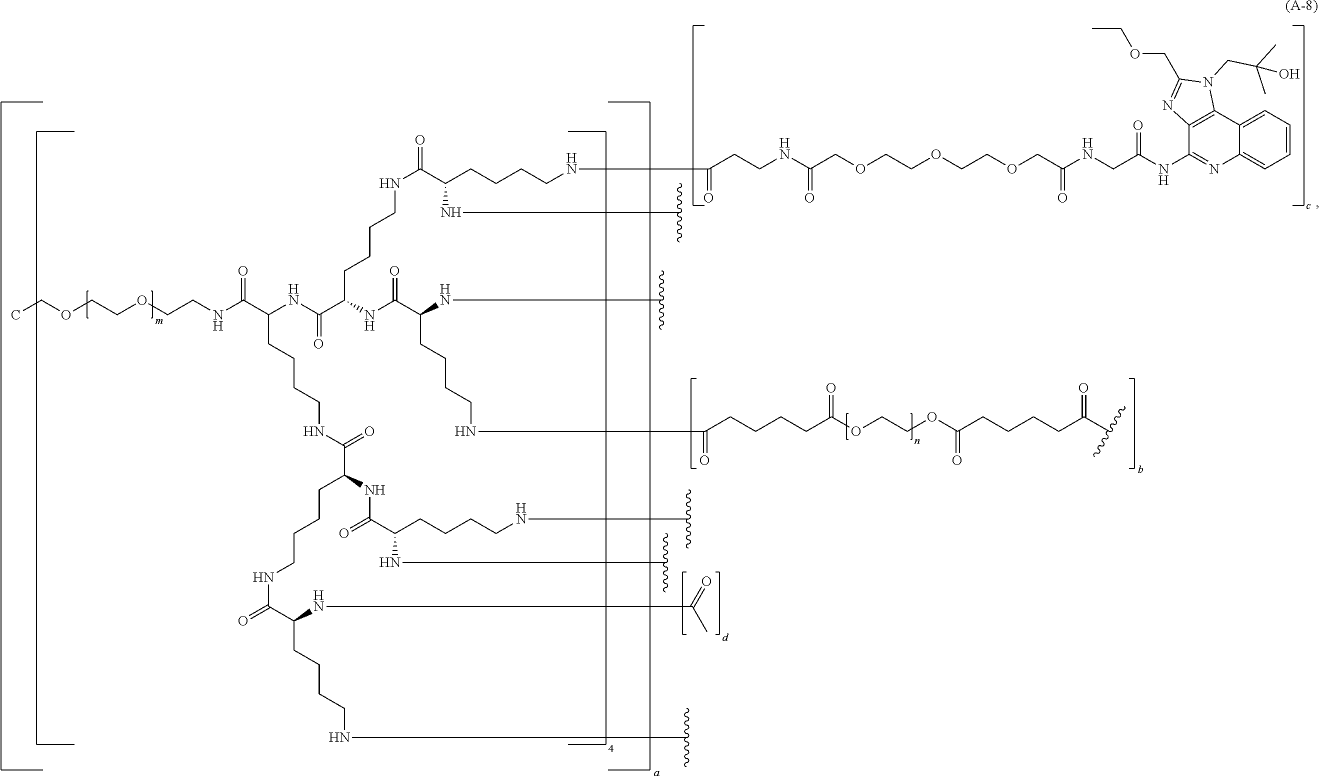

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:3:3.

In a fourth aspect the present invention relates to a conjugate of formula (A-3).

In a fifth aspect the present invention relates to a conjugate of formula (A-6). In certain embodiments the conjugate of the fifth aspect as a ratio of “a”:“b”:“c”:“d” of approx. 1:13:2.4:3.6.

In a sixth aspect the present invention relates to a conjugate of formula (A-7).

In a seventh aspect the present invention relates to a conjugate of formula (A-8).

In an eighth aspect the present invention relates to a conjugate of formula (Ai-6).

In a ninth aspect the present invention relates to a pharmaceutical formulation comprising one or more TLR7/8 agonist conjugate of formula (Ai-6), (A-6), (A-7) or (A-8) or a pharmaceutically acceptable salt thereof. In certain embodiments such pharmaceutical formulation is a dry formulation, such as a lyophilized formulation. In certain embodiments such pharmaceutical formulation is a suspension.

In certain embodiments the pharmaceutical formulation comprises the TLR7/8 agonist conjugate or a pharmaceutically acceptable salt thereof in a concentration ranging from 0.5 mg to 2 mg TLR7/8 agonist per ml. In certain embodiments the pharmaceutical formulation comprises the TLR7/8 agonist conjugate or a pharmaceutically acceptable salt thereof in a concentration of 1 mg TLR7/8 agonist per ml. In certain embodiments the pharmaceutical formulation comprises the TLR7/8 agonist conjugate or a pharmaceutically acceptable salt thereof in a concentration of 2 mg TLR7/8 agonist per ml.

In certain embodiments the conjugate of the fourth, fifth, sixth, seventh or eighth aspect or a pharmaceutically acceptable salt thereof or the pharmaceutical formulation of the ninth aspect is for use as a medicament. In certain embodiments such medicament is for the treatment of cancer, in particular a solid tumor. In certain embodiments such solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.

In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC). In certain embodiments the solid tumor is selected from the group consisting of pancreatic cancer, prostate cancer, melanoma, SCCHN, cutaneous squamous cell cancer (cSCC) and cervical cancer. In certain embodiments the solid tumor is a pancreatic cancer. In certain embodiments the solid tumor is prostate cancer. In certain embodiments the solid tumor is a melanoma. In certain embodiments the solid tumor is a SCCHN. In certain embodiments the solid tumor is a cSCC. In certain embodiments the solid tumor is cervical cancer. In certain embodiments the medicament is administered via intratumoral injection, such as by using a fanning technique.

In certain embodiments such medicament is administered to a patient at a dose ranging from 0.3 mg to 3 mg of TLR7/8 agonist per tumor. In certain embodiments such medicament is administered at a dose ranging from 0.3 mg to 3 mg of TLR7/8 agonist per tumor via intratumoral injection. In certain embodiment the medicament is administered at a dose of 0.5 mg of TLR7/8 agonist per tumor. In certain embodiment the medicament is administered at a dose of 0.5 mg of TLR7/8 agonist per tumor via intratumoral injection. In certain embodiment the medicament is administered at a dose of 1 mg of TLR7/8 agonist per tumor. In certain embodiment the medicament is administered at a dose of 1 mg of TLR7/8 agonist per tumor via intratumoral injection. In certain embodiments the medicament is administered using a fanning technique.

In certain embodiments the medicament is administered to a patient every two to four weeks. In certain embodiments the medicament is administered to a patient every three weeks. In certain embodiments the medicament is administered to a patient every two weeks. In certain embodiments the medicament is administered to a patient every four weeks.

In certain embodiments the medicament is administered to a patient every five weeks. In certain embodiments the medicament is administered to a patient every six weeks. In certain embodiments the medicament is administered to a patient every seven weeks. In certain embodiments the medicament is administered to a patient every eight weeks. In certain embodiments the medicament is administered to a patient every nine weeks. In certain embodiments the medicament is administered to a patient every ten weeks. In certain embodiments the medicament is administered to a patient every twelve weeks. In certain embodiments the medicament is administered to a patient every six months. In certain embodiments the medicament is administered to a patient every seven months. In certain embodiments the medicament is administered to a patient every eight months. In certain embodiments the medicament is administered to a patient every nine months. In certain embodiments the medicament is administered to a patient every ten months. In certain embodiments the medicament is administered to a patient every eleven months. In certain embodiments the medicament is administered to a patient once a year. In certain embodiments the medicament is administered to a patient at a frequency dependent on disease progression.

In certain embodiments the medicament is administered to one tumor of the patient. In certain embodiments the medicament is administered to more than one tumor of the patient, such as to two tumors, three tumors, four tumors or five tumors or to all tumors of a patient that have a sufficient size and are accessible for treatment. In certain embodiments treatment of one tumor leads to an abscopal effect in one or more untreated tumors.

In certain embodiments the medicament is administered to a patient in a cotreatment with an inhibitor if PD-1 or an inhibitor of PD-L1, which may be given prior to, together with or after administration of the medicament.

In certain embodiments the medicament is administered to a patient in a cotreatment with an inhibitor of PD-1, which may be given prior to, together with or after administration of the medicament. An inhibitor of PD-1 may be selected from the group consisting of pembrolizumab, nivolumab, pidilizumab, AMP-224, BMS-936559, cemiplimab and PDR001. An inhibitor of PD-L1 may be selected from the group consisting of MDX-1105, MEDI4736, atezolizumab, avelumab, BMS-936559 and durvalumab. In certain embodiments the inhibitor of PD-1 is pembrolizumab. Details regarding the administration of pembrolizumab are as described elsewhere herein.

In certain embodiments the medicament is provided in a vial. Such vial may be a vial with a stopper, such as a bromo-butyl stopper, and a seal cap, such as an aluminium seal cap. In certain embodiments the vial is a glass vial, such as a type 1 glass vial. Such a vial may have a size of 1 ml, 2 ml or 5 ml, for example. In certain embodiments the vial has a size of 2 ml. In certain embodiments the vial has a size of 1 ml. In certain embodiments the vial has a size of 5 ml. In certain embodiments such vial comprises a unit dose. Such unit dose comprises in certain embodiments 0.5 mg of TLR7/8 agonist. In certain embodiments the unit dose is a liquid, such as a suspension, which has a volume ranging from 0.1 to 1.5 ml. In certain embodiments the unit dose is provided as a suspension and has a volume of 0.5 ml.

Embodiments for the tumor to which the medicament is administered are as described elsewhere herein.

In certain embodiments the conjugate of the fourth, fifth, sixth, seventh or eighth aspect or the pharmaceutical formulation of the ninth aspect is for use in the treatment of cancer, such as of a solid tumor.

In certain embodiments such solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.

In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC). In certain embodiments the solid tumor is selected from the group consisting of pancreatic cancer, prostate cancer, melanoma, SCCHN, cutaneous squamous cell cancer (cSCC) and cervical cancer. In certain embodiments the solid tumor is a pancreatic cancer. In certain embodiments the solid tumor is prostate cancer. In certain embodiments the solid tumor is a melanoma. In certain embodiments the solid tumor is a SCCHN. In certain embodiments the solid tumor is a cSCC. In certain embodiments the solid tumor is cervical cancer. In certain embodiments the medicament is administered via intratumoral injection, such as by using a fanning technique.

In certain embodiments the conjugate of the fourth, fifth, sixth, seventh or eighth aspect or the pharmaceutical formulation of the ninth aspect is for use in the treatment of cancer, such as a solid tumor, and is administered at a dose ranging from 0.3 mg to 3 mg of TLR7/8 agonist per tumor, such as at a dose of 0.5 mg of TLR7/8 agonist per tumor. In certain embodiments administration is a dose of 1 mg of TLR7/8 agonist per tumor. In certain embodiments administration is a dose of 2 mg of TLR7/8 agonist per tumor.

In certain embodiments the conjugate of the fourth, fifth, sixth or seventh aspect or the pharmaceutical formulation of the eighth aspect is for use in the treatment of cancer and is administered to a patient every two to four weeks, such as for example every three weeks. In certain embodiments administration of the conjugate or the pharmaceutical formulation to a patient occurs every two weeks. In certain embodiments administration of the conjugate or the pharmaceutical formulation to a patient occurs every four weeks.

In certain embodiments administration of the conjugate or the pharmaceutical formulation to a patient occurs every five weeks. In certain embodiments administration of the conjugate or the pharmaceutical formulation to a patient occurs every six weeks. In certain embodiments administration of the conjugate or the pharmaceutical formulation to a patient occurs every every seven weeks. In certain embodiments administration of the conjugate or the pharmaceutical formulation to a patient occurs every every eight weeks. In certain embodiments administration of the conjugate or the pharmaceutical formulation to a patient occurs every nine weeks. In certain embodiments administration of the conjugate or the pharmaceutical formulation to a patient occurs every ten weeks. In certain embodiments administration of the conjugate or the pharmaceutical formulation to a patient occurs every twelve weeks. In certain embodiments administration of the conjugate or the pharmaceutical formulation to a patient occurs every six months. In certain administration of the conjugate or the pharmaceutical formulation to a patient occurs every seven months. In certain embodiments administration of the conjugate or the pharmaceutical formulation to a patient occurs every eight months. In certain embodiments administration of the conjugate or the pharmaceutical formulation to a patient occurs every nine months. In certain embodiments administration of the conjugate or the pharmaceutical formulation to a patient occurs every ten months. In certain embodiments administration of the conjugate or the pharmaceutical formulation to a patient occurs every eleven months. In certain embodiments administration of the conjugate or the pharmaceutical formulation to a patient occurs once a year. In certain embodiments administration of the conjugate or the pharmaceutical formulation to a patient occurs at a frequency dependent on disease progression.

In certain embodiments the conjugate of the fourth, fifth, sixth, seventh or eighth aspect or the pharmaceutical formulation of the ninth aspect is for use in the treatment of cancer and the conjugate or the pharmaceutical formulation is administered to one tumor of a patient. In certain embodiments the conjugate of the fourth, fifth, sixth, seventh or eighth aspect or the pharmaceutical formulation of the ninth aspect is for use in the treatment of cancer and the conjugate or the pharmaceutical formulation is administered to more than one tumor of the patient, such as to two tumors, three tumors, four tumors or five tumors or to all tumors of a patient that have a sufficient size and are accessible for treatment. In certain embodiments treatment of one tumor leads to an abscopal effect in one or more untreated tumors.

In certain embodiments the conjugate of the fourth, fifth, sixth, seventh or eighth aspect or the pharmaceutical formulation of the ninth aspect is for use in the treatment of cancer and the treatment is a cotreatment with an inhibitor of PD-1 or an inhibitor of PD-L1, which may be given prior to, together with or after administration of the conjugate or the pharmaceutical formulation. In certain embodiments the treatment is a cotreatment with an inhibitor of PD-1, which may be given prior to, together with or after administration of the conjugate or the pharmaceutical formulation. Such inhibitor of PD-1 may be selected from the group consisting of pembrolizumab, nivolumab, pidilizumab, AMP-224, BMS-936559, cemiplimab and PDR001 and is in certain embodiments pembrolizumab. Details regarding the administration of pembrolizumab are as described elsewhere herein.

In certain embodiments the conjugate of the fourth, fifth, sixth, seventh or eighth aspect or the pharmaceutical formulation of the ninth aspect is for use in the treatment of cancer and the TLR7/8 agonist conjugate or a pharmaceutically acceptable salt thereof or the pharmaceutical formulation is provided in a vial. Such vial may be a vial with a stopper, such as a bromo-butyl stopper, and a seal cap, such as an aluminium seal cap. In certain embodiments the vial is a glass vial, such as a type 1 glass vial. Such a vial may have a size of 1 ml, 2 ml or 5 ml, for example. In certain embodiments the vial has a size of 2 ml. In certain embodiments the vial has a size of 1 ml. In certain embodiments the vial has a size of 5 ml. In certain embodiments such vial comprises a unit dose.

In certain embodiments the conjugate of the fourth, fifth, sixth, seventh or eighth aspect or the pharmaceutical formulation of the ninth aspect is administered at a dose ranging from 0.3 mg to 3 mg per tumor and said dose is provided in a volume ranging from from 0.1 ml to 2 ml. In certain embodiments the conjugate of the fourth, fifth, sixth, seventh or eighth aspect or the pharmaceutical formulation of the ninth aspect is administered at a dose ranging from 0.3 mg to 3 mg per tumor and said dose is provided in a volume ranging from 0.2 ml to 1.5 ml. In certain embodiments the conjugate of the fourth, fifth, sixth, seventh or eighth aspect or the pharmaceutical formulation of the ninth aspect is administered at a dose ranging from 0.3 mg to 3 mg per tumor and said dose is provided of 0.25 ml. In certain embodiments the conjugate of the fourth, fifth, sixth, seventh or eighth aspect or the pharmaceutical formulation of the ninth aspect is administered at a dose ranging from 0.3 mg to 3 mg per tumor and said dose is provided in a volume of 0.5 ml. In certain embodiments the conjugate of the fourth, fifth, sixth, seventh or eighth aspect or the pharmaceutical formulation of the ninth aspect is administered at a dose ranging from 0.3 mg to 3 mg per tumor and said dose is provided in a volume of 1 ml.

In certain embodiments the conjugate of the fourth, fifth, sixth, seventh or eighth aspect or the pharmaceutical formulation of the ninth aspect is administered at a dose 0.5 mg per tumor and said dose is provided in a volume ranging from from 0.1 ml to 2 ml. In certain embodiments the conjugate of the fourth, fifth, sixth, seventh or eighth aspect or the pharmaceutical formulation of the ninth aspect is administered at a dose of 0.5 mg per tumor and said dose is provided in a volume ranging from 0.2 ml to 1.5 ml. In certain embodiments the conjugate of the fourth, fifth, sixth, seventh or eighth aspect or the pharmaceutical formulation of the ninth aspect is administered at a dose of 0.5 mg per tumor and said dose is provided of 0.25 ml. In certain embodiments the conjugate of the fourth, fifth, sixth, seventh or eighth aspect or the pharmaceutical formulation of the ninth aspect is administered at a dose of 0.5 mg per tumor and said dose is provided in a volume of 0.5 ml. In certain embodiments the conjugate of the fourth, fifth, sixth, seventh or eighth aspect or the pharmaceutical formulation of the ninth aspect is administered at a dose of 0.5 mg per tumor and said dose is provided in a volume of 1 ml.

In certain embodiments the conjugate of the fourth, fifth, sixth, seventh or eighth aspect or the pharmaceutical formulation of the ninth aspect is administered at a dose 1 mg per tumor and said dose is provided in a volume ranging from from 0.1 ml to 2 ml. In certain embodiments the conjugate of the fourth, fifth, sixth, seventh or eighth aspect or the pharmaceutical formulation of the ninth aspect is administered at a dose of 1 mg per tumor and said dose is provided in a volume ranging from 0.2 ml to 1.5 ml. In certain embodiments the conjugate of the fourth, fifth, sixth, seventh or eighth aspect or the pharmaceutical formulation of the ninth aspect is administered at a dose of 1 mg per tumor and said dose is provided of 0.25 ml. In certain embodiments the conjugate of the fourth, fifth, sixth, seventh or eighth aspect or the pharmaceutical formulation of the ninth aspect is administered at a dose of 1 mg per tumor and said dose is provided in a volume of 0.5 ml. In certain embodiments the conjugate of the fourth, fifth, sixth, seventh or eighth aspect or the pharmaceutical formulation of the ninth aspect is administered at a dose of 1 mg per tumor and said dose is provided in a volume of 1 ml.

In certain embodiments the conjugate of the fourth, fifth, sixth or seventh aspect is for use in the treatment of a solid tumor, wherein the conjugate is administered via intratumoral injection using a fanning technique at a dose of 0.5 mg per tumor.

In certain embodiments the conjugate of the fourth, fifth, sixth, seventh or eighth aspect or the pharmaceutical formulation of the ninth aspect is for use in the treatment of a solid tumor, wherein the conjugate is administered every three weeks via intratumoral injection at a dose of 0.5 mg per tumor.

In certain embodiments the conjugate of the fourth, fifth, sixth, seventh or eighth aspect or the pharmaceutical formulation of the ninth aspect is for use in the treatment of a solid tumor, wherein the conjugate is administered via intratumoral injection at a dose of 0.5 mg per tumor and wherein the treatment is a cotreatment with pembrolizumab.

In certain embodiments the conjugate the fourth, fifth, sixth, seventh or eighth aspect or the pharmaceutical formulation of the ninth aspect is for use in the treatment of a solid tumor, wherein the conjugate is administered via intratumoral injection at a dose of 0.5 mg per tumor and wherein the treatment is a cotreatment with pembrolizumab, wherein pembrolizumab is administered every three weeks, either prior to, together with or after administration of the conjugate of the fourth, fifth, sixth, seventh or eighth aspect or the pharmaceutical formulation of the ninth aspect.

In certain embodiments the conjugate of the fourth, fifth, sixth, seventh or eighth aspect or the pharmaceutical formulation of the ninth aspect is for use in the treatment of a solid tumor, wherein the conjugate is administered via intratumoral injection at a dose of 0.5 mg per tumor and wherein the treatment is a cotreatment with pembrolizumab, wherein pembrolizumab is administered every three weeks via intravenous injection at a dose of 200 mg, either prior to, together with or after administration of the conjugate of the fourth, fifth, sixth, seventh or eighth aspect or the pharmaceutical formulation of the ninth aspect.

In certain embodiments the conjugate of the fourth, fifth, sixth, seventh or eighth aspect or the pharmaceutical formulation of the ninth aspect is for use in the treatment of a solid tumor, wherein the conjugate is administered via intratumoral injection using a fanning technique at a dose of 0.5 mg per tumor and wherein the treatment is a cotreatment with pembrolizumab, wherein pembrolizumab is administered every three weeks via intravenous injection at a dose of 200 mg, either prior to, together with or after administration of the conjugate the fourth, fifth, sixth, seventh or eighth aspect or the pharmaceutical formulation of the ninth aspect.

In certain embodiments the conjugates of fourth, fifth, sixth, seventh or eighth aspect or the pharmaceutical formulation of the ninth aspect are for use in the manufacture of a medicament. In certain embodiments the medicament is for the treatment of cancer, such as for the treatment of a solid tumor.

In certain embodiments such solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.

In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC). In certain embodiments the solid tumor is selected from the group consisting of pancreatic cancer, prostate cancer, melanoma, SCCHN, cutaneous squamous cell cancer (cSCC) and cervical cancer. In certain embodiments the solid tumor is a pancreatic cancer. In certain embodiments the solid tumor is prostate cancer. In certain embodiments the solid tumor is a melanoma. In certain embodiments the solid tumor is a SCCHN. In certain embodiments the solid tumor is a cSCC. In certain embodiments the solid tumor is cervical cancer. In certain embodiments the medicament is administered via intratumoral injection, such as by using a fanning technique.

In certain embodiments such medicament is administered to a patient at a dose ranging from 0.3 mg to 3 mg of TLR7/8 agonist per tumor. In certain embodiments such medicament is administered at a dose ranging from 0.3 mg to 3 mg of TLR7/8 agonist per tumor via intratumoral injection. In certain embodiment the medicament is administered at a dose of 0.5 mg of TLR7/8 agonist per tumor. In certain embodiment the medicament is administered at a dose of 0.5 mg of TLR7/8 agonist per tumor via intratumoral injection. In certain embodiment the medicament is administered at a dose of 1 mg of TLR7/8 agonist per tumor. In certain embodiment the medicament is administered at a dose of 1 mg of TLR7/8 agonist per tumor via intratumoral injection. In certain embodiments the medicament is administered via intratumoral injection. In certain embodiments the medicament is administered using a fanning technique.

In certain embodiments the medicament is administered to a patient every two to four weeks. In certain embodiments the medicament is administered to a patient every three weeks. In certain embodiments the medicament is administered to a patient every two weeks. In certain embodiments the medicament is administered to a patient every four weeks.

In certain embodiments the medicament is administered to a patient every five weeks. In certain embodiments the medicament is administered to a patient every six weeks. In certain embodiments the medicament is administered to a patient every seven weeks. In certain embodiments the medicament is administered to a patient every eight weeks. In certain embodiments the medicament is administered to a patient every nine weeks. In certain embodiments the medicament is administered to a patient every ten weeks. In certain embodiments the medicament is administered to a patient every twelve weeks. In certain embodiments the medicament is administered to a patient every six months. In certain embodiments the medicament is administered to a patient every seven months. In certain embodiments the medicament is administered to a patient every eight months. In certain embodiments the medicament is administered to a patient every nine months. In certain embodiments the medicament is administered to a patient every ten months. In certain embodiments the medicament is administered to a patient every eleven months. In certain embodiments the medicament is administered to a patient once a year. In certain embodiments the medicament is administered to a patient at a frequency dependent on disease progression.

In certain embodiments the medicament is administered to one tumor of the patient. In certain embodiments the medicament is administered to more than one tumor of the patient, such as to two tumors, three tumors, four tumors or five tumors or to all tumors of a patient that have a sufficient size and are accessible for treatment. In certain embodiments treatment of one tumor leads to an abscopal effect in one or more untreated tumors.

In certain embodiments the medicament is administered to a patient in a cotreatment with an inhibitor if PD-1 or an inhibitor of PD-L1, which may be given prior to, together with or after administration of the medicament.

In certain embodiments the medicament is administered to a patient in a cotreatment with an inhibitor of PD-1, which may be given prior to, together with or after administration of the medicament. An inhibitor of PD-1 may be selected from the group consisting of pembrolizumab, nivolumab, pidilizumab, AMP-224, BMS-936559, cemiplimab and PDR001. An inhibitor of PD-L1 may be selected from the group consisting of MDX-1105, MEDI4736, atezolizumab, avelumab, BMS-936559 and durvalumab. In certain embodiments the inhibitor of PD-1 is pembrolizumab. Details regarding the administration of pembrolizumab are as described elsewhere herein.

In certain embodiments the medicament is provided in a vial. Such vial may be a vial with a stopper, such as a bromo-butyl stopper, and a seal cap, such as an aluminium seal cap. In certain embodiments the vial is a glass vial, such as a type 1 glass vial. Such a vial may have a size of 1 ml, 2 ml or 5 ml, for example. In certain embodiments the vial has a size of 2 ml. In certain embodiments the vial has a size of 1 ml. In certain embodiments the vial has a size of 5 ml. In certain embodiments such vial comprises a unit dose. Such unit dose comprises in certain embodiments 0.5 mg of TLR7/8 agonist. In certain embodiments the unit dose is a liquid, such as a suspension, which has a volume ranging from 0.1 to 1.5 ml. In certain embodiments the unit dose is provided as a suspension and has a volume of 0.5 ml.

In another aspect the present invention relates to a method of treating a patient having cancer, wherein the method comprises the step of administering a pharmaceutically effective amount of the TLR7/8 agonist conjugate of the fourth, fifth, sixth, seventh or eighth aspect or the pharmaceutical formulation of the ninth aspect to the patient. In certain embodiments the cancer is a solid tumor. In certain embodiments the cancer is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.

In certain embodiments the solid tumor is selected from the group consisting of squamous cell carcinoma of the head and neck (SCCHN); HPV-associated cancers, such as anal, vulvar, cervical, penile and vaginal cancers; melanomas; pancreatic cancer and breast cancer, such as triple-negative breast cancer (TNBC). In certain embodiments the solid tumor is selected from the group consisting of pancreatic cancer, prostate cancer, melanoma, SCCHN, cutaneous squamous cell cancer (cSCC) and cervical cancer. In certain embodiments the solid tumor is a pancreatic cancer. In certain embodiments the solid tumor is prostate cancer. In certain embodiments the solid tumor is a melanoma. In certain embodiments the solid tumor is a SCCHN. In certain embodiments the solid tumor is a cSCC. In certain embodiments the solid tumor is cervical cancer. In certain embodiments the medicament is administered via intratumoral injection, such as by using a fanning technique.

In certain embodiments the conjugate of the fourth, fifth, sixth, seventh or eighth aspect or the pharmaceutical formulation of the ninth aspect is administered to the patient at a dose ranging from 0.3 mg to 3 mg of TLR7/8 agonist per tumor, such as at a dose of 0.5 mg of TLR7/8 agonist per tumor. In certain embodiments administration is a dose of 1 mg of TLR7/8 agonist per tumor. In certain embodiments administration is a dose of 2 mg of TLR7/8 agonist per tumor. Such dose is a pharmaceutically effective amount or pharmaceutically effective dose.

In certain embodiments the conjugate of the fourth, fifth, sixth or seventh aspect or the pharmaceutical formulation of the eighth aspect is for use in the treatment of cancer and is administered to the patient every two to four weeks, such as for example every three weeks. In certain embodiments administration of the conjugate or the pharmaceutical formulation to the patient occurs every two weeks. In certain embodiments administration of the conjugate or the pharmaceutical formulation to the patient occurs every four weeks.

In certain embodiments administration of the conjugate or the pharmaceutical formulation to the patient occurs every five weeks. In certain embodiments administration of the conjugate or the pharmaceutical formulation to the patient occurs every six weeks. In certain embodiments administration of the conjugate or the pharmaceutical formulation to the patient occurs every every seven weeks. In certain embodiments administration of the conjugate or the pharmaceutical formulation to the patient occurs every every eight weeks. In certain embodiments administration of the conjugate or the pharmaceutical formulation to the patient occurs every nine weeks. In certain embodiments administration of the conjugate or the pharmaceutical formulation to the patient occurs every ten weeks. In certain embodiments administration of the conjugate or the pharmaceutical formulation to a patient occurs every twelve weeks. In certain embodiments administration of the conjugate or the pharmaceutical formulation to the patient occurs every six months. In certain administration of the conjugate or the pharmaceutical formulation to the patient occurs every seven months. In certain embodiments administration of the conjugate or the pharmaceutical formulation to the patient occurs every eight months. In certain embodiments administration of the conjugate or the pharmaceutical formulation to the patient occurs every nine months. In certain embodiments administration of the conjugate or the pharmaceutical formulation to the patient occurs every ten months. In certain embodiments administration of the conjugate or the pharmaceutical formulation to the patient occurs every eleven months. In certain embodiments administration of the conjugate or the pharmaceutical formulation to the patient occurs once a year. In certain embodiments administration of the conjugate or the pharmaceutical formulation to the patient occurs at a frequency dependent on disease progression.

In certain embodiments the conjugate of the fourth, fifth, sixth, seventh or eighth aspect or the pharmaceutical formulation of the ninth aspect is administered to one tumor of the patient. In certain embodiments the conjugate of the fourth, fifth, sixth, seventh or eighth aspect or the pharmaceutical formulation of the ninth aspect is administered to more than one tumor of the patient, such as to two tumors, three tumors, four tumors or five tumors or to all tumors of the patient that have a sufficient size and are accessible for treatment. In certain embodiments treatment of one tumor leads to an abscopal effect in one or more untreated tumors.

In certain embodiments the method of treatment is a cotreatment with an inhibitor of PD-1 or an inhibitor of PD-L1, which may be given prior to, together with or after administration of the conjugate or the pharmaceutical formulation. In certain embodiments the method is a cotreatment with an inhibitor of PD-1, which may be given prior to, together with or after administration of the conjugate or the pharmaceutical formulation. Such inhibitor of PD-1 may be selected from the group consisting of pembrolizumab, nivolumab, pidilizumab, AMP-224, BMS-936559, cemiplimab and PDR001 and is in certain embodiments pembrolizumab. Details regarding the administration of pembrolizumab are as described elsewhere herein.

In certain embodiments the conjugate of the fourth, fifth, sixth, seventh or eighth aspect or the pharmaceutical formulation of the ninth aspect is provided in a vial. Such vial may be a vial with a stopper, such as a bromo-butyl stopper, and a seal cap, such as an aluminium seal cap. In certain embodiments the vial is a glass vial, such as a type 1 glass vial. Such a vial may have a size of 1 ml, 2 ml or 5 ml, for example. In certain embodiments the vial has a size of 2 ml. In certain embodiments the vial has a size of 1 ml. In certain embodiments the vial has a size of 5 ml. In certain embodiments such vial comprises a unit dose.

In a further aspect the present invention relates to a TLR7/8 agonist conjugate for use in the treatment of cancer, in particular of a solid tumor, wherein administration of a pharmaceutically effective dose leads to a plasma concentration of the TLR7/8 agonist of less than 4000 μg/ml, such as of less than 2000 μg/ml, of less than 1000 μg/ml, of less than 750 μg/ml or of less than 500 μg/ml. In certain embodiments the TLR7/8 agonist conjugate is administered via intratumoral injection, such as by using a fanning technique. In certain embodiments the TLR7/8 agonist conjugate is administered at a dose ranging from 0.3 to 3 mg of the TLR7/8 agonist, such as at a dose of 0.5 mg of TLR7/8 agonist or 1 mg of TLR7/8 agonist. In certain embodiments the TLR7/8 agonist conjugate is administered to a patient every 2 to 4 weeks, such as every 3 weeks. In certain embodiments the TLR7/8 agonist conjugate is administered in a cotreatment with an inhibitor of PD-1 or PD-L1, such as pembrolizumab.

The TLR7/8 agonist conjugate, examples for solid tumors, doses, administration frequencies, volumes and details regarding the cotreatment are as described elsewhere herein.
(01) A TLR7/8 agonist conjugate or a pharmaceutically acceptable salt thereof of formula (Ai-6):

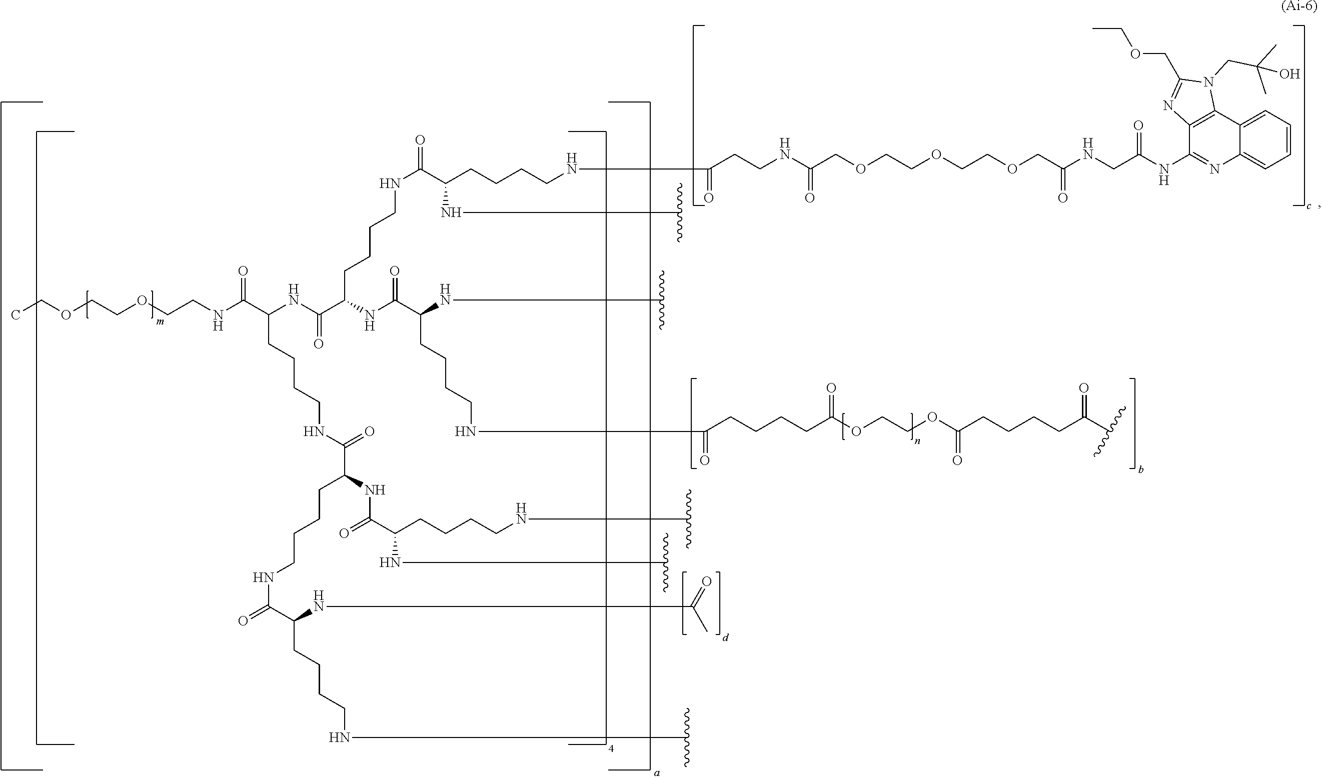

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:c:d with c+d=6.
(02) The TLR7/8 agonist or a pharmaceutically acceptable salt thereof of paragraph (01), wherein the ratio of moieties “a”:“b”:“c”:“d” in the conjugate is approx. 1:13:3:3.
(03) The TLR7/8 agonist conjugate or a pharmaceutically acceptable salt thereof of paragraph (01), wherein the ratio of moieties “a”:“b”:“c”:“d” in the conjugate is approx. 1:13:2:4.
(04) The TLR7/8 agonist conjugate or a pharmaceutically acceptable salt thereof of paragraph (01), wherein the ratio of moieties “a”:“b”:“c”:“d” in the conjugate is approx. 1:13:4:2.
(05) A pharmaceutical formulation comprising one or more TLR7/8 agonist conjugate or a pharmaceutically acceptable salt thereof of any one of paragraphs (01) to (04).
(06) The pharmaceutical formulation of paragraph (05), wherein pharmaceutical formulation is a dry formulation.
(07) The pharmaceutical formulation of paragraph (05) or (06), wherein the pharmaceutical formulation is a lyophilized formulation.
(08) The pharmaceutical formulation of paragraph (05), wherein the pharmaceutical formulation is a suspension.
(09) The pharmaceutical formulation of paragraph (08), wherein the pharmaceutical formulation comprises the TLR7/8 agonist conjugate or a pharmaceutically acceptable salt thereof in a concentration ranging from 0.5 mg to 2 mg TLR7/8 agonist per ml.
(10) The pharmaceutical formulation of paragraph (08) or (09), wherein the pharmaceutical formulation comprises the TLR7/8 agonist conjugate or a pharmaceutically acceptable salt thereof in a concentration of 1 mg TLR7/8 agonist per ml.
(11) The TLR7/8 agonist conjugate or a pharmaceutically acceptable salt thereof of any one of paragraphs (01) to (04) or the pharmaceutical formulation of any one of paragraphs (05) to (10) for use as a medicament.
(12) The TLR7/8 agonist conjugate or a pharmaceutically acceptable salt thereof or the pharmaceutical formulation for use of paragraph (11), wherein the medicament is for the treatment of cancer, such as a solid tumor.
(13) The TLR7/8 agonist conjugate or a pharmaceutically acceptable salt thereof or the pharmaceutical formulation for use of paragraph (12), wherein the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.
(14) The TLR7/8 agonist conjugate or a pharmaceutically acceptable salt thereof or the pharmaceutical formulation for use of paragraph (12) or (13), wherein the cancer is selected from the group consisting of squamous cell carcinomas of the head and neck (SCCHN), HPV-associated cancers, melanomas, pancreatic cancer and breast cancer.
(15) The TLR7/8 agonist conjugate or a pharmaceutically acceptable salt thereof or the pharmaceutical formulation for use of any one of paragraphs (12) to (14), wherein the medicament is administered at a dose ranging from 0.3 mg to 3 mg of TLR7/8 agonist per tumor.
(16) The TLR7/8 agonist conjugate or a pharmaceutically acceptable salt thereof or the pharmaceutical formulation for use of any one of paragraphs (12) to (15), wherein the medicament is administered at a dose of 0.5 mg of TLR7/8 agonist per tumor.
(17) The TLR7/8 agonist conjugate or a pharmaceutically acceptable salt thereof or the pharmaceutical formulation for use of any one of paragraphs (11) to (16), wherein the medicament is administered to a patient every two to four weeks.
(18) The TLR7/8 agonist conjugate or a pharmaceutically acceptable salt thereof or the pharmaceutical formulation for use of any one of paragraphs (11) to (17), wherein the medicament is administered to a patient every three weeks.
(19) The TLR7/8 agonist conjugate or a pharmaceutically acceptable salt thereof or the pharmaceutical formulation for use of any one of paragraphs (12) to (19), wherein the medicament is administered via intratumoral injection.
(20) The TLR7/8 agonist conjugate or a pharmaceutically acceptable salt thereof or the pharmaceutical formulation for use of paragraph (19), wherein the medicament is administered via intratumoral injection using a fanning technique.
(21) The TLR7/8 agonist conjugate or a pharmaceutically acceptable salt thereof or the pharmaceutical formulation for use of any one of paragraphs (12) to (20), wherein the medicament is administered to one tumor of the patient.
(22) The TLR7/8 agonist conjugate or a pharmaceutically acceptable salt thereof or the pharmaceutical formulation for use of any one of paragraphs (12) to (21), wherein the medicament is administered to more than one tumor of the patient, such as to two tumors, three tumors, four tumors or five tumors or to all tumors of a patient that have a sufficient size and are accessible for treatment.
(23) The TLR7/8 agonist conjugate or a pharmaceutically acceptable salt thereof or the pharmaceutical formulation for use of any one of paragraphs (12) to (22), wherein treatment of one tumor leads to an abscopal effect in one or more untreated tumors.
(24) The TLR7/8 agonist conjugate or a pharmaceutically acceptable salt thereof or the pharmaceutical formulation for use of any one of paragraphs (12) to (23), wherein medicament is administered to a patient in a cotreatment with an inhibitor of PD-1 or an inhibitor of PD-L1, which may be given prior to, together with or after administration of the medicament.
(25) The TLR7/8 agonist conjugate or a pharmaceutically acceptable salt thereof or the pharmaceutical formulation for use of any one of paragraphs (12) to (24), wherein the medicament is administered to a patient in is a cotreatment with an inhibitor of PD-1, which may be given prior to, together with or after administration of the medicament.
(26) The TLR7/8 agonist conjugate or a pharmaceutically acceptable salt thereof or the pharmaceutical formulation for use of paragraph (24) or (25), wherein the inhibitor of PD-1 is selected from the group consisting of pembrolizumab, nivolumab, pidilizumab, AMP-224, BMS-936559, cemiplimab and PDR001.
(27) The TLR7/8 agonist conjugate or a pharmaceutically acceptable salt thereof or the pharmaceutical formulation for use of any one of paragraphs (24) to (26), wherein the inhibitor of PD-1 is pembrolizumab.
(28) The TLR7/8 agonist conjugate or a pharmaceutically acceptable salt thereof or the pharmaceutical formulation for use of any one of paragraphs (11) to (27), wherein the medicament is provided in a vial.
(29) The TLR7/8 agonist conjugate or a pharmaceutically acceptable salt thereof or the pharmaceutical formulation for use of paragraph (28), wherein the vial is a type 1 glass vial.
(30) The TLR7/8 agonist conjugate or a pharmaceutically acceptable salt thereof or the pharmaceutical formulation for use of paragraph (28) or (29), wherein the vial has a size of 1 ml, 2 ml or 5 ml.
(31) The TLR7/8 agonist conjugate or a pharmaceutically acceptable salt thereof of any one of paragraphs (01) to (04) or the pharmaceutical formulation of any one of paragraphs (05) to (10) for use in the treatment of cancer, such as of a solid tumor.
(32) The TLR7/8 agonist conjugate or a pharmaceutically acceptable salt thereof or the pharmaceutical formulation for use of paragraph (31), wherein the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.
(33) The TLR7/8 agonist conjugate or a pharmaceutically acceptable salt thereof or the pharmaceutical formulation for use of paragraph (31) or (32), wherein the cancer is selected from the group consisting of squamous cell carcinoma of the head and neck (SCCHN), HPV-associated cancers, melanomas, pancreatic cancer and breast cancer.
(34) The TLR7/8 agonist conjugate or a pharmaceutically acceptable salt thereof or the pharmaceutical formulation for use of any one of paragraphs (31) to (33), wherein the TLR7/8 agonist conjugate or a pharmaceutically acceptable salt thereof or the pharmaceutical formulation is administered at a dose ranging from 0.3 mg to 3 mg of TLR7/8 agonist per tumor.
(35) The TLR7/8 agonist conjugate or a pharmaceutically acceptable salt thereof or the pharmaceutical formulation for use of any one of paragraphs (31) to (34), wherein the TLR7/8 agonist conjugate or a pharmaceutically acceptable salt thereof or the pharmaceutical formulation is administered at a dose of 0.5 mg of TLR7/8 agonist per tumor.
(36) The TLR7/8 agonist conjugate or a pharmaceutically acceptable salt thereof or a pharmaceutically acceptable salt thereof or the pharmaceutical formulation for use of any one of paragraphs (31) to (35), wherein the TLR7/8 agonist or a pharmaceutically acceptable salt thereof or the pharmaceutical formulation is administered to a patient every two to four weeks.
(37) The TLR7/8 agonist conjugate or a pharmaceutically acceptable salt thereof or the pharmaceutical formulation for use of any one of paragraphs (31) to (36), wherein the TLR7/8 agonist or a pharmaceutically acceptable salt thereof or the pharmaceutical formulation is administered to a patient every three weeks.
(38) The TLR7/8 agonist conjugate or a pharmaceutically acceptable salt thereof or the pharmaceutical formulation for use of any one of paragraphs (31) to (37), wherein the TLR7/8 agonist or the pharmaceutical formulation is administered via intratumoral injection.
(39) The TLR7/8 agonist conjugate or a pharmaceutically acceptable salt thereof or the pharmaceutical formulation for use of any one of paragraphs (31) to (38), wherein the TLR7/8 agonist or a pharmaceutically acceptable salt thereof or the pharmaceutical formulation is administered via intratumoral injection using a fanning technique.
(40) The TLR7/8 agonist conjugate or a pharmaceutically acceptable salt thereof or the pharmaceutical formulation for use of any one of paragraphs (31) to (39), wherein the TLR7/8 agonist conjugate or a pharmaceutically acceptable salt thereof or the pharmaceutical formulation is administered to one tumor of a patient.
(41) The TLR7/8 agonist conjugate or a pharmaceutically acceptable salt thereof or the pharmaceutical formulation for use of any one of paragraphs (31) to (39), wherein the TLR7/8 agonist conjugate or a pharmaceutically acceptable salt thereof or the pharmaceutical formulation is administered to more than one tumor of the patient, such as to two tumors, three tumors, four tumors or five tumors or to all tumors of a patient that have a sufficient size and are accessible for treatment.
(42) The TLR7/8 agonist conjugate or a pharmaceutically acceptable salt thereof or the pharmaceutical formulation for use of any one of paragraphs (31) to (41), wherein treatment of one tumor leads to an abscopal effect in one or more untreated tumors.
(43) The TLR7/8 agonist conjugate or a pharmaceutically acceptable salt thereof or the pharmaceutical formulation for use of any one of paragraphs (31) to (42), wherein the treatment is a cotreatment with an inhibitor of PD-1 or an inhibitor of PD-L1, which may be given prior to, together with or after administration of the TLR7/8 agonist conjugate or a pharmaceutically acceptable salt thereof or the pharmaceutical formulation.
(44) The TLR7/8 agonist conjugate or a pharmaceutically acceptable salt thereof or the pharmaceutical formulation for use of any one of paragraphs (31) to (43), wherein the treatment is a cotreatment with an inhibitor of PD-1, which may be given prior to, together with or after administration of the TLR7/8 agonist conjugate or a pharmaceutically acceptable salt thereof or the pharmaceutical formulation.
(45) The TLR7/8 agonist conjugate or a pharmaceutically acceptable salt thereof or the pharmaceutical formulation for use of paragraph (43) or (44), wherein the inhibitor of PD-1 is selected from the group consisting of pembrolizumab, nivolumab, pidilizumab, AMP-224, BMS-936559, cemiplimab and PDR001.
(46) The TLR7/8 agonist conjugate or a pharmaceutically acceptable salt thereof or the pharmaceutical formulation for use of any one of paragraphs (43) to (45), wherein the inhibitor of PD-1 is pembrolizumab.
(47) The TLR7/8 agonist conjugate or a pharmaceutically acceptable salt thereof or the pharmaceutical formulation for use of any one of paragraphs (31) to (46), wherein the TLR7/8 agonist conjugate or a pharmaceutically acceptable salt thereof or the pharmaceutical formulation is provided in a vial.
(48) The TLR7/8 agonist conjugate or a pharmaceutically acceptable salt thereof or the pharmaceutical formulation for use of paragraph (47), wherein the vial is a type 1 glass vial.
(49) The TLR7/8 agonist conjugate or a pharmaceutically acceptable salt thereof or the pharmaceutical formulation for use of paragraph (47) or (48), wherein the vial has a size of 1 ml, 2 ml or 5 ml.
(50) The TLR7/8 agonist conjugate or a pharmaceutically acceptable salt thereof of any one of paragraphs (01) to (04) or the pharmaceutical formulation of any one of paragraphs (05) to (10) for use in the manufacture of a medicament.
(51) The TLR7/8 agonist conjugate or a pharmaceutically acceptable salt thereof or the pharmaceutical formulation for use of paragraph (50), wherein the medicament is for the treatment of cancer, such as for the treatment of a solid tumor.
(52) The TLR7/8 agonist conjugate or a pharmaceutically acceptable salt thereof or the pharmaceutical formulation for use of paragraph (51), wherein the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.
(53) The TLR7/8 agonist conjugate or a pharmaceutically acceptable salt thereof or the pharmaceutical formulation for use of paragraph (51) or (52), wherein the cancer is selected from the group consisting of squamous cell carcinoma of the head and neck (SCCHN), HPV-associated cancers, melanomas, pancreatic cancer and breast cancer.
(54) The TLR7/8 agonist conjugate or a pharmaceutically acceptable salt thereof or the pharmaceutical formulation for use of any one of paragraphs (51) to (53), wherein the medicament is administered at a dose ranging from 0.3 mg to 3 mg of TLR7/8 agonist per tumor.
(55) The TLR7/8 agonist conjugate or a pharmaceutically acceptable salt thereof or the pharmaceutical formulation for use of any one of paragraphs (51) to (54), wherein the medicament is administered at a dose of 0.5 mg of TLR7/8 agonist per tumor.
(56) The TLR7/8 agonist conjugate or a pharmaceutically acceptable salt thereof or the pharmaceutical formulation for use of any one of paragraphs (50) to (55), wherein the medicament is administered to a patient every two to four weeks.
(57) The TLR7/8 agonist conjugate or a pharmaceutically acceptable salt thereof or the pharmaceutical formulation for use of any one of paragraphs (50) to (56), wherein the medicament is administered to a patient every three weeks.
(58) The TLR7/8 agonist conjugate or a pharmaceutically acceptable salt thereof or the pharmaceutical formulation for use of any one of paragraphs (51) to (57), wherein the medicament is administered via intratumoral injection.
(59) The TLR7/8 agonist conjugate or a pharmaceutically acceptable salt thereof or the pharmaceutical formulation for use of paragraph (58), wherein the medicament is administered via intratumoral injection using a fanning technique.
(60) The TLR7/8 agonist conjugate or a pharmaceutically acceptable salt thereof or the pharmaceutical formulation for use of any one of paragraphs (51) to (59), wherein the medicament is administered to one tumor of the patient.
(61) The TLR7/8 agonist conjugate or a pharmaceutically acceptable salt thereof or the pharmaceutical formulation for use of any one of paragraphs (51) to (59), wherein the medicament is administered to more than one tumor of the patient, such as to two tumors, three tumors, four tumors or five tumors or to all tumors of a patient that have a sufficient size and are accessible for treatment.
(62) The TLR7/8 agonist conjugate or a pharmaceutically acceptable salt thereof or the pharmaceutical formulation for use of any one of paragraphs (51) to (61), wherein treatment of one tumor leads to an abscopal effect in one or more untreated tumors.
(63) The TLR7/8 agonist conjugate or a pharmaceutically acceptable salt thereof or the pharmaceutical formulation for use of any one of paragraphs (51) to (62), wherein the medicament is administered to a patient in a cotreatment with an inhibitor of PD-1 or an inhibitor of PD-L1, which may be given prior to, together with or after administration of the medicament.
(64) The TLR7/8 agonist conjugate or a pharmaceutically acceptable salt thereof or the pharmaceutical formulation for use of any one of paragraphs (51) to (63), wherein the is administered to a patient in a cotreatment with an of PD-1, which may be given prior to, together with or after administration of the medicament.
(65) The TLR7/8 agonist conjugate or a pharmaceutically acceptable salt thereof or the pharmaceutical formulation for use of paragraph (63) or (64), wherein the inhibitor of PD-1 is selected from the group consisting of pembrolizumab, nivolumab, pidilizumab, AMP-224, BMS-936559, cemiplimab and PDR001.
(66) The TLR7/8 agonist conjugate or a pharmaceutically acceptable salt thereof or the pharmaceutical formulation for use of any one of paragraphs (63) to (65), wherein the inhibitor of PD-1 is pembrolizumab.
(67) The TLR7/8 agonist conjugate or a pharmaceutically acceptable salt thereof or the pharmaceutical formulation for use of any one of paragraphs (50) to (66), wherein the medicament is provided in a vial.
(68) The TLR7/8 agonist conjugate or a pharmaceutically acceptable salt thereof or the pharmaceutical formulation for use of paragraph (67), wherein the vial is a type 1 glass vial.
(69) The TLR7/8 agonist conjugate or a pharmaceutically acceptable salt thereof or the pharmaceutical formulation for use of paragraph (67) or (68), wherein the vial has a size of 1 ml, 2 ml or 5 ml.
(70) A method of treating a patient having cancer, wherein the method comprises the step of administering a pharmaceutically effective amount of the TLR7/8 agonist conjugate or a pharmaceutically acceptable salt thereof of any one of paragraphs (01) to (04) or the pharmaceutical formulation of any one of paragraphs (05) to (10) to the patient.
(71) The method of paragraph (70), wherein the cancer is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.
(72) The method of paragraph (70) or (71), wherein the cancer is selected from the group consisting of squamous cell carcinoma of the head and neck (SCCHN), HPV-associated cancers, melanomas, pancreatic cancer and breast cancer.
(73) The method of any one of paragraphs (70) to (72), wherein the TLR7/8 agonist conjugate or a pharmaceutically acceptable salt thereof or the pharmaceutical formulation is administered to the patient at a dose ranging from 0.3 mg to 3 mg of TLR7/8 agonist per tumor.
(74) The method of any one of paragraphs (70) to (73), wherein the TLR7/8 agonist conjugate or a pharmaceutically acceptable salt thereof or the pharmaceutical formulation is administered to the patient at a dose of 0.5 mg of TLR7/8 agonist per tumor.
(75) The method of any one of paragraphs (70) to (74), wherein the TLR7/8 agonist or a pharmaceutically acceptable salt thereof or the pharmaceutical formulation is administered to a patient every two to four weeks.
(76) The method of any one of paragraphs (70) to (75), wherein the TLR7/8 agonist or a pharmaceutically acceptable salt thereof or the pharmaceutical formulation is administered to a patient every three weeks.
(77) The method of any one of paragraphs (70) to (76), wherein the TLR7/8 agonist or a pharmaceutically acceptable salt thereof or the pharmaceutical formulation is administered via intratumoral injection.
(78) The method of any one of paragraphs (70) to (77), wherein the TLR7/8 agonist or a pharmaceutically acceptable salt thereof or the pharmaceutical formulation is administered via intratumoral injection using a fanning technique.
(79) The method of any one of paragraphs (70) to (78), wherein the TLR7/8 agonist or a pharmaceutically acceptable salt thereof or the pharmaceutical formulation is administered to one tumor of the patient.
(80) The method of any one of paragraphs (70) to (78), wherein the TLR7/8 agonist or a pharmaceutically acceptable salt thereof or the pharmaceutical formulation is administered to more than one tumor of the patient, such as to two tumors, three tumors, four tumors or five tumors or to all tumors of a patient that have a sufficient size and are accessible for treatment.
(81) The method of any one of paragraphs (70) to (80), wherein treatment of one tumor leads to an abscopal effect in one or more untreated tumors.
(82) The method of any one of paragraphs (70) to (81), wherein the TLR7/8 agonist conjugate or a pharmaceutically acceptable salt thereof or the pharmaceutical formulation is administered in a cotreatment with an inhibitor of PD-1 or an inhibitor of PD-L1, which may be given prior to, together with or after administration of the TLR7/8 agonist conjugate or a pharmaceutically acceptable salt thereof or the pharmaceutical formulation.
(83) The method of any one of paragraphs (70) to (82), wherein the TLR7/8 agonist conjugate or a pharmaceutically acceptable salt thereof or the pharmaceutical formulation is administered in a cotreatment with an inhibitor of PD-1, which may be given prior to, together with or after administration of the TLR7/8 agonist conjugate or a pharmaceutically acceptable salt thereof or the pharmaceutical formulation.
(84) The method of paragraph (82) or (83), wherein the inhibitor of PD-1 is selected from the group consisting of pembrolizumab, nivolumab, pidilizumab, AMP-224, BMS-936559, cemiplimab and PDR001.
(85) The method for use of any one of paragraphs (82) to (84), wherein the inhibitor of PD-1 is pembrolizumab.
(86) The method of any one of paragraphs (70) to (85), wherein the TLR7/8 agonist conjugate or a pharmaceutically acceptable salt thereof or the pharmaceutical formulation is provided in a vial.
(87) The method of paragraph (86), wherein the vial is a type 1 glass vial.
(88) The method of paragraph (86) or (87), wherein the vial has a size of 1 ml, 2 ml or 5 ml.
(89) A TLR7/8 agonist conjugate for use in the treatment of cancer, in particular of a solid tumor, wherein administration of a pharmaceutically effective dose leads to a plasma concentration of the TLR7/8 agonist of less than 4000 μg/ml.
(90) The TLR7/8 agonist conjugate for use of paragraph (89), wherein the plasma concentration of the TLR7/8 agonist is less than 2000 μg/ml.
(91) The TLR7/8 agonist conjugate for use of paragraph (89) or (90), wherein the plasma concentration of the TLR7/8 agonist is less than 1000 μg/ml.
(92) The TLR7/8 agonist conjugate for use of any one of paragraphs (89) to (91), wherein the plasma concentration of the TLR7/8 agonist is less than 750 μg/ml.
(93) The TLR7/8 agonist conjugate for use of any one of paragraphs (89) to (92), wherein the plasma concentration of the TLR7/8 agonist is less than 500 μg/ml.
(94) The TLR7/8 agonist conjugate for use of any one of paragraphs (89) to (93), wherein the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.
(95) The TLR7/8 agonist conjugate of any one of paragraphs (89) to (94), wherein the solid tumor is selected from the group consisting of squamous cell carcinoma of the head and neck (SCCHN), HPV-associated cancers, melanomas, pancreatic cancer and breast cancer.
(96) The TLR7/8 agonist conjugate of any one of paragraphs (89) to (95), wherein the TLR7/8 agonist conjugate is administered via intratumoral injection.
(97) The TLR7/8 agonist conjugate of paragraph (96), wherein the intratumoral injection is via a fanning technique.
(98) The TLR7/8 agonist conjugate of any one of paragraphs (89) to (97), wherein the TLR7/8 agonist conjugate is administered every two to four weeks.
(99) The TLR7/8 agonist conjugate of any one of paragraphs (89) to (98), wherein the TLR7/8 agonist conjugate is administered every three weeks.
(100) The TLR7/8 agonist conjugate of any one of paragraphs (89) to (99), wherein the TLR7/8 agonist is administered at a dose ranging from 0.3 mg to 3 mg of TLR7/8 agonist per tumor.
(101) The TLR7/8 agonist conjugate of any one of paragraphs (89) to (100), wherein the TLR7/8 agonist conjugate is administered at a dose of 0.5 mg of TLR7/8 agonist per tumor.

### EXAMPLES

Compound 1 has the following structure:

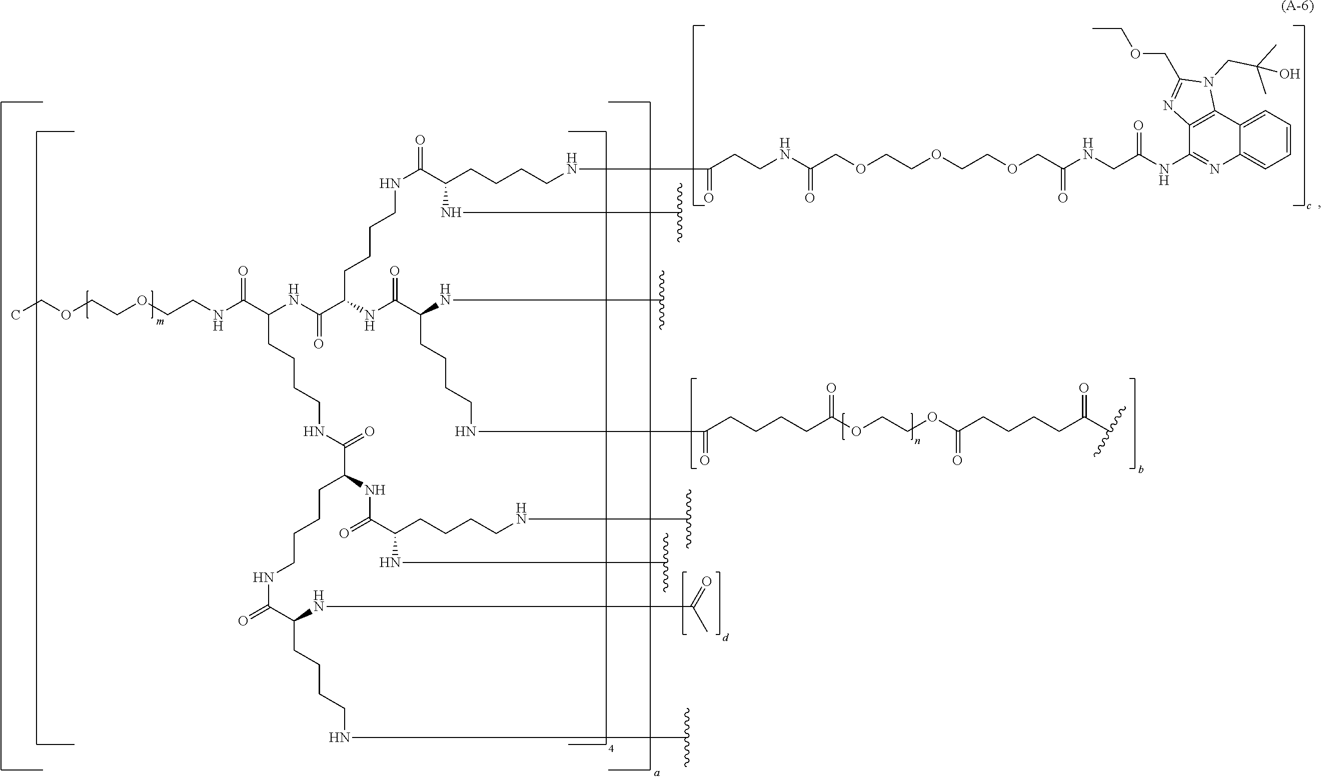

wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:2:4;
   and can be manufactured as described for hydrogel 14 in WO2020/141221.

Note: Experiments and resulting data are reported in past tense, even though at the time of filing the clinical trial was ongoing.

### Example 1

Data shown herein is from a Phase 1/2, open-label, multi-center, first-in-human dose escalation and dose expansion study of compound 1, delivered by IT injection, for participants aged 18 years and older with locally advanced or metastatic solid tumors. The clinical trial is listed on www.clinicaltrials.gov under the ClinicalTrials.gov Identifier: NCT04799054.

Participants must have:
At least 2 measurable lesions;
Lesion(s) for IT injection must be between 20 mm and 50 mm (inclusive) in the longest diameter when selected for injection; and
Lesion(s) for IT injection must be easily and safely accessible (for example, cutaneous or subcutaneous, palpable, and/or lesions visualized by ultrasound).

The study uses a standard 3+3 design to investigate a range of dose levels of compound 1 as monotherapy or in combination with pembrolizumab.

### Part 1: Monotherapy Dose Escalation

Part 1 included participants with advanced solid tumors that have progressed on or were intolerant of available standard of care treatment options or have disease for which there is no standard of care therapy. Compound 1 starting dose (Dose Level 1) was administered IT every 3 weeks. Upon disease progression, the addition of pembrolizumab every 3 weeks was allowed if deemed appropriate by the investigator.

Part 2: Combination Dose Escalation with Pembrolizumab

Part 2 started after Part 1 Dose Level 1 has passed safety evaluation. Compound 1 was given in a staggered dosing schedule in combination with pembrolizumab at 200 mg in Cycle 1 only (specifically, pembrolizumab is given 7 days after compound 1). After Cycle 1 (28 days), compound 1 was given together with pembrolizumab at 200 mg every 3 weeks in subsequent cycles (21 days).

For Part 1 and Part 2: There were at least 48 hours after dosing of the first participant before any subsequent participant is enrolled in a given cohort. This allows for evaluation of the first participant (defined as sentinel participant) in each dose cohort for any potential serious acute local or systemic reactions.

Doses are summarized in Table 1.

TABLE 1
Dose levels of Parts 1 and 2

| Compound 1 (Parts 1 and 2)¹ | | | | |
|---|---|---|---|---|
| | Dose | Number of | Total | Pembrolizumab |
| | Per | Injected | Injected | (for Part |
| Dose Level³ | Lesion⁴ | Lesions | Dose/Patient | 2)⁵ |
| Dose level 1 | 0.3 | 1 | 0.3 mg | 200 mg Q3W |
| Dose level 2 | 0.5 | Up to 2 | 0.5-1.0 mg | |
| Dose level 3 | 1 | Up to 3 | 1.0-3.0 mg | |
| Dose level 4 | 2 | Up to 3 | 2.0-6.0 mg | |
| Dose level 5 | 4 | Up to 3 | 4.0-12.0 mg | |

Abbreviations: mg = milligram; Q3W = every 3 weeks.
¹All lesions chosen for intratumoral injections in an individual participant were treated at the same procedure setting on the same day.
³Dose levels refer to resiquimod equivalent dose levels with the dose being administered per tumor site. With the exception of Dose Level 1, dose levels for subsequent cohorts may have been adjusted (escalated or de-escalated) based on clinical and available PK/PD observations. Each level was evaluated by the Safety Review Committee (SRC) after safety and available PK/PD data from the DLT period of a minimum of 3 participants enrolled to receive a dose level.
⁴Compound 1 was given Q3W in Part 1 and Cycle 2 onwards in Part 2 (of note, Cycle 1 in Part 2 is 28 days long due to staggered dosing of Compound 1 and pembrolizumab)
⁵If dose limiting toxicities were observed at any one dose level, dose may have been de-escalated in the same cohort or the previous dose level may have been reopened to enrollment to ensure the availability of safety and PK/PD data for a minimum of 6 participants for RP2D decision. Upon disease progression in Part 1, the addition of pembrolizumab was permitted at the discretion of the Investigator and after discussion with and approval from the Medical Monitor.

### Part 3: Combination Dose Expansion (Phase 2)

In Part 3, compound 1 was given at the RP2D determined from Part 2 along with pembrolizumab at 200 mg every 3 weeks (Q3W). The number of indication-specific dose expansion cohorts was determined by a combination of clinical data seen from Parts 1 and 2, treatment landscape for a certain indication, and/or compelling scientific rationale.

Number of Participants: Approximately 40 participants were enrolled in total for Parts 1 and 2. Each expansion cohort in Part 3 was based on Simon-2-stage design and enrolled up to approximately 50 participants, depending on benchmark ORR data in each indication. Table 2 shows the patient disposition.

TABLE 2
Patient disposition. Tumor types and number
of prior lines of anti-cancer therapy

| | Monotherapy | Combination |
|---|---|---|
| | (n = 5) | (n = 3) |
| Tumor types | Melanoma (2) | Melanoma (2) |
| (# prior lines | Melanoma (3) | Basal Cell Carcinoma (2) |
| of anti-cancer | Melanoma (3) | Pancreatic (2) |
| therapy) | TNBC (5) | |
| | Pancreatic (5) | |
| Prior anti-PD(L)1 | 4 (80%) | 2 (67%) |

### Results

To date (December 2021), no dose-limiting toxicities were observed. In the safety-evaluable population (n=8) so far there was no indication of systemic side effects related to compound 1. Of note, the only related adverse event reported was transient injection site related reactions (Grade 1/2). This was reported in 2 out of 5 patients treated with monotherapy, and no injection site reactions have been reported so far for the 3 patients treated with combination treatment. Table 3 provides the safety summary.

TABLE 3
Safety Summary

| | Monotherapy | | Combination | |
|---|---|---|---|---|
| Overview of Safety | (n = 5) | | (n = 3) | |
| Subjects with at least 1 AE | 4 (80%) | | 2 (67%) | |
| Subjects with at least | 3 (60%) | Related: 0 | 1 (33%) | Related: 0 |
| one SAE | | | | |
| Subjects with at least | 3 (60%) | Related: 0 | 1 (33%) | Related: 0 |
| one >=3 AE | | | | |
| Subjects with an AE leading | 0 | | 0 | |
| to study drug withdrawn | | | | |
| or study discontinuation | | | | |

| | | |
|---|---|---|
| Subjects with Death | 0 | 0 |
| related to AE | | |

### Efficacy:

| | Tumor type | | Best Overall Response |
|---|---|---|---|
| | (# prior lines | | per RECIST v1.1 |
| | of anti-cancer | Prior | (latest assessment), |
| | therapy) | PD(L)1 | study status |
| Monotherapy | Melanoma (2) | yes | uPR (week 27), |
| (dose level 1) | | | ongoing |
| Combination with | Pancreatic | no | SD (week 9), |
| pembro (dose | cancer (2) | | ongoing |
| level 1) | | | |
| Combination with | Basal cell | yes | SD (week 9), |
| pembro (dose | carcinoma (2) | | ongoing |
| level 1) | | | |

### Melanoma Patient: uPR (Week 27)

| Target lesions | Baseline | wk 9 | wk 18 | wk 27 |
|---|---|---|---|---|
| Injected (mm) | 25 | 24 | 24 | 30 |
| Non-injected (mm) | 11 | 10 | 9 | 0 |

3 core biopsies of injected lesion at week 27 after 9 cycles showed tumor found in up to 50% of total tissue; focal foreign material (hydrogel carrier) was surrounded by granulomatous inflammation.

Punch biopsy at non-injected lesion location showed mild chronic inflammation, reactive changes, no evidence of malignancy.

### Pancreatic Cancer SD (Week 9)

| Target lesions | Baseline | wk 9 |
|---|---|---|
| Injected once in | 22 | 30 |
| Cycle 1 (mm) | | |
| Non-injected (mm) | 90 | |
| Injected since | | 90 |
| Cycle 2 (mm) | | |

3 core biopsies of injected lesion at week 7 after 1 dose showed no tumor present; minimal lymphohistiocytic reaction was observed.

### Basal Cell Carcinoma Patient SD (Week 9)

| Target lesions | Baseline | wk 9 |
|---|---|---|
| Injected once in | 42 | 47.5 |
| Cycle 1 (mm) | | |
| Non-injected (mm) | 17 | 18.4 |

3 mm punch biopsy (5 mm deep) of injected lesion at week 9 after 3 cycles showed atypical basaloid proliferation at base of biopsy. No evidence of malignancy were detected.

### PK Measurements

Resiquimod systemic concentration was determined following IT administration of compound 1 at a dose of 0.3 and 0.5 mg per lesion. The mean systemic half-life of resiquimod released from the intratumoral administered depot was 6.5 days. Cₘₐₓ values were generally below 150 μg/mL and well below levels reported in the literature to be associated with cytokine release syndrome (˜4000 μg/mL). Table 4 show the results of the resiquimod plasma concentration measurements.

TABLE 4
Resiquimod plasma concentration measurements

| Dose | Time | | Plasma conc | SD |
|---|---|---|---|---|
| (μg) | (day) | N | (pg/mL) | (pg/mL) |
| 300 | 0 | 6 | | |
| | 0.021 | 6 | 63.7 | 38.8 |
| | 0.083 | 6 | 63.7 | 31.5 |
| | 0.167 | 6 | 63.2 | 43.7 |
| | 1 | 6 | 52.5 | 72.2 |
| | 3 | 6 | 51.5 | 59.3 |
| | 7 | 6 | 49.1 | 67.8 |
| | 14 | 5 | 33.6 | 58.9 |
| | 21 | 5 | 20.2 | 40.9 |
| 500 | 0 | 1 | <LLOQ | |
| | 0.021 | 1 | 14.5 | |
| | 0.083 | 1 | 32.4 | |
| | 1 | 1 | 62.1 | |
| | 3 | 1 | 121 | |
| | 7 | 1 | 85.7 | |
| | 14 | 1 | 47.4 | |
| | 21 | 1 | 38.8 | |

### Resiquimod and O-Desethyl Resiquimod:

Plasma samples were spiked with an internal standard (IS) (resiquimod-d5 and O-desethyl resiquimod-d6) followed by supported liquid extraction (SLE) with ethyl acetate using SLE+ plates. The analytes were then separated from remaining endogenous compounds on an Acquity UPLC HSS T3 C18 (50×2.1 mm, 1.8 μm) and analyzed using positive electrospray ionization (ESI) tandem mass spectrometry (MS/MS) in multiple reaction monitoring mode (MRM). The resiquimod and O-desethyl resiquimod calibration range is 2.00 to 2,500 μg/mL. The concentrations of resiquimod and O-desethyl resiquimod in plasma were calculated using weighed (1/X²) linear regression from plasma calibration standards.

### Gene Expression

Gene expression was evaluated by NanoString® technology using the Nanostring nCounter® Human Immunology V2 Panel. Tumor biopsies collected prior to first dosing with compound 1 (pre-dose) and 7 days after first dosing (C1D8) were formalin fixed and paraffin embedded followed by analysis. The absolute gene expression counts were adjusted by technical negative and positive controls and normalized to Housekeeping genes (genes that have a consistent and stable expression level) contained in the nCounter® Human Immunology V2 Panel. Fold change in expression from pre-dose to C1D8 expression was calculated and log 2 transformed.

Patients were treated with intratumoral injection of compound 1 dosed into a single tumor lesion at either 0.3 mg per lesion (Dose level 1) or 0.5 mg per lesion (Dose level 2).

| Patient | 1 | 2 | 3 | 4 | 5 | 6 |
|---|---|---|---|---|---|---|
| IRF7 | 1.7 | 3.5 | 3.5 | 4.1 | 3.5 | 1.8 |
| CXCL10 | 0.6 | 6.7 | 7.2 | 12 | 1.4 | 3.6 |
| STAT1 | 0.5 | 3.5 | 4.4 | 3.4 | 0.5 | 2.7 |
| STAT2 | 1.2 | 2.6 | 3 | 1.4 | 0.9 | 0.9 |
| IRF1 | −0.6 | 2.5 | 1.7 | 0.8 | 2 | 2.3 |
| IRF3 | −0.1 | 0.3 | 4.9 | −1 | 1.9 | 0.8 |
| IRF4 | −0.1 | 1.5 | 6.3 | 0.8 | 0.8 | −0.1 |
| IRF5 | −0.7 | 2.4 | 4.7 | 0.2 | 1.2 | 1.2 |
| IRF8 | −0.6 | 2.9 | 1.7 | 1.9 | −0.4 | 0.3 |
| IFNB1 | 0.5 | 1 | 2.4 | 5.7 | −0.1 | −0.3 |
| IFNA1/13 | −0.4 | 1.5 | −2.9 | −0.4 | −0.1 | −0.1 |
| IFNA2 | −0.2 | 0.7 | −3.3 | −1.7 | 3.8 | −0.3 |
| JAK1 | −0.5 | 0.4 | 0.5 | 0.6 | 0.6 | 0.7 |

Table showing log2 fold changes of tumoral gene expression 7 days post first compound 1 dose. (>0 = increased expression, 0 = no change, <0 = decreased expression)

### Biomarker Measurements

Gene expression was evaluated by NanoString® technology using the Nanostring nCounter® Human Immunology V2 Panel. Tumor biopsies collected prior to first dosing with compound 1 (pre-dose) and 7 days after first dosing (C1D8) were formalin fixed and paraffin embedded followed by analysis. The absolute gene expression counts were adjusted by technical negative and positive controls and normalized to Housekeeping genes (genes that have a consistent and stable expression level) contained in the nCounter® Human Immunology V2 Panel. Fold change in expression from pre-dose to C1D8 expression was calculated and log 2 transformed.

Patients were treated with intratumoral injection of compound 1 dosed into a single tumor lesion at either 0.3 mg per lesion (Dose level 1) or 0.5 mg per lesion (Dose level 2). 3 weeks thereafter patients were treated with intratumoral injection of compound 1 dosed into a single tumor lesion at either 0.3 mg per lesion (Dose level 1) or 0.5 mg per lesion (Dose level 2) In addition, selected patients received intravenous treatment with pembrolizumab dosed at 200 mg per patient 1 week after the first dose of compound 1 and 3 weeks after the first dose of compound 1.

Sustained immune activation following repeat administration of compound 1 was observed. Gene expression remained elevated after second administration, indicating no down-regulation of immune activation.

In conclusion, compound 1 was administered at 300 μg and 500 μg per lesion to patients with solid tumors. At these dose levels signs of activity in three out of three evaluable patients including those previously treated with checkpoint inhibitors were observed. Compound 1 demonstrated monotherapy activity and consistent and robust target engagement, as measured by biomarkers.

Compound 1 was well-tolerated, with transient, mild injection site-related reactions (Grade 1/2) are the only related AEs. In patients treated with compound 1, no systemic side effects related to compound 1 was reported, consistent with low systemic exposure of resiquimod.

Compound 1 has the potential for sustained immune activation and systemic anti-tumor response with infrequent dosing using dose levels of 300 μg and 500 μg per lesion.

### Updated Results:

As of Sep. 21, 2022, 23 patients were enrolled in the dose escalation: 9 to monotherapy and 14 to combination therapy. The following results are based on this data cutoff and may differ from the December 2021 data as more information is gathered.

### Patient Baseline Demographics and Clinical Characteristics

### Monotherapy (Part 1)

| | Dose | | |
|---|---|---|---|
| | 0.3 mg/lesion | 0.5 mg/lesion | Total |
| Demographics | (n = 3) | (n = 6) | (N = 9) |
| Age (years), median (min, max) | 60 (58, 66) | 65 (42, 75) | 63 (42, 75) |
| Sex | | | |
| Male | 2 | 4 | 6 |
| Female | 1 | 2 | 3 |
| Race | | | |
| Asian | 0 | 1 | 1 |
| Black | 1 | 0 | 1 |
| White | 2 | 5 | 7 |
| Ethnicity | | | |
| Hispanic or Latino | 0 | 1 | 1 |
| Not Hispanic or Latino | 3 | 5 | 8 |
| ECOG Performance Status | | | |
| Grade 0 | 2 | 2 | 4 |
| Grade 1 | 1 | 4 | 5 |
| Prior Anti-PD1 Therapy (Yes), n (%) | 2 (66.7) | 4 (66.7) | 6 (66.7) |
| Number of Prior Lines of Systemic | 5 (2, 5) | 3 (2, 4) | 3 (2, 5) |
| Therapy, median (min, max) | | | |
| Tumor Types | | | |
| Head and Neck Squamous Cell | 0 | 1 | 1 |
| Melanoma | 1 | 2 | 3 |
| Pancreatic Cancer | 1 | 1 | 2 |
| Cutaneous Squamous Cell | 0 | 1 | 1 |
| Other | 1 | 1 | 2 |

Abbreviation: ECOG Eastern Cooperative Oncology Group

Combination with Pembrolizumab (Part 2)

| | Dose | | |
|---|---|---|---|
| | 0.3 mg/lesion | 0.5 mg/lesion | Total |
| Demographics | (n = 3) | (n = 11) | (N = 14) |
| Age (years), median (min, max) | 49 (47, 66) | 70 (43, 86) | 69 (43, 86) |
| Sex | | | |
| Male | 1 | 7 | 8 |
| Female | 2 | 4 | 6 |
| Race | | | |
| Asian | 0 | 0 | 0 |
| Black | 0 | 0 | 0 |
| White | 3 | 11 | 14 |
| Ethnicity | | | |
| Hispanic or Latino | 0 | 1 | 1 |
| Not Hispanic or Latino | 3 | 10 | 13 |
| ECOG Performance Status | | | |
| Grade 0 | 2 | 2 | 4 |
| Grade 1 | 1 | 9 | 10 |
| Prior Anti-PD1 Therapy (Yes), n (%) | 2 (66.7) | 7 (63.6) | 9 (64.3) |
| Number of Prior Lines of Systemic | 2 (2, 2) | 2 (1, 3) | 2 (1, 3) |
| Therapy, median (min, max) | | | |
| Tumor Types | | | |
| Head and Neck Squamous Cell | 0 | 2 | 2 |
| Melanoma | 1 | 2 | 3 |
| Pancreatic Cancer | 1 | 0 | 1 |
| Cutaneous Squamous Cell | 0 | 1 | 1 |
| Other | 1 | 6 | 7 |

Abbreviation: ECOG Eastern Cooperative Oncology Group

### Safety

Compound 1 was well tolerated as monotherapy and in combination with pembrolizumab at 5 both 0.3 mg/lesion and 0.5 mg/lesion doses. All treatment related adverse events to compound 1 were grades 1 and 2, except one grade 3 injection site reaction. One dose-limiting toxicity (Grade 3 injection site reaction) was observed in a patient receiving 0.5 mg/lesion and pembrolizumab.

TABLE 4
Safety Summary

| | Monotherapy | | Combination with Pembrolizumab | |
|---|---|---|---|---|
| | (Part1) | | (Part 2) | |
| | 0.3 mg/lesion | 0.5 mg/lesion | 0.3 mg/lesion | 0.5 mg/lesion |
| | (n = 3) | (n = 6) | (n = 3) | (n = 11) |
| Patients with ≥ | 0 | 0 | 1 | 1 |
| related SAE or | | | (Grade 3 | (SAE: Grade 3 |
| related grade 3 | | | hyperglycemia | injection site |
| or higher TEAE | | | related to | reaction) |
| | | | pembrolizumab) | |
| TEAE leading to | 1 | 1 | 0 | 2 |
| study drug | (Grade 3 | (Grade 3 | | (Grade 3 seizure |
| interruption | gluteal pain | aspiration | | not related to |
| any cycle | not related to | pneumonia | | compound 1; 1, |
| | compoud 1) | not related to | | grade 3 injection |
| | | compound 1) | | site reaction |
| | | | | related to |
| | | | | compound 1) |
| TEAE leading to | 0 | 0 | 0 | 0 |
| compound 1 | | | | |
| withdrawn | | | | |
| TEAE leading | 0 | 0 | 0 | 0 |
| to death | | | | |

Abbreviations:
SAE serious adverse events;
TEAE treatment emergent adverse events

Efficacy: Preliminary efficacy data show clinical response to compound 1 occurred in 2 out of 13 response evaluable patients with one abscopal (non-injected lesion) effect.

TABLE 5
Best Overall Response

| | | Prior | Best Overall |
|---|---|---|---|
| | Tumor | anti- | Response per |
| Study Drug | type | PD(L)1therapy | RECIST v 1.1 |
| Monotherapy | Melanoma | yes | PR with abscopal |
| (0.3 mg/lesion) | | | effect (based on |
| | | | pathology review) |
| Monotherapy | Melanoma | yes | NE |
| (0.5 mg/lesion | | | |
| Monotherapy | Melanoma | yes | SD |
| (0.5 mg/lesion) | | | |
| Monotherapy | Head and Neck | yes | SD |
| (0.5 mg/lesion) | Squamous Cell | | |
| Monotherapy | Pancreatic | no | PD |
| (0.5 mg/lesion) | | | |
| Monotherapy | Colon Mixed | no | PD |
| (0.5 mg/lesion) | Adenoneuroendocrine | | |
| Combination with | Basel Cell | yes | SD |
| pembrolizumab | Carcinoma | | |
| (0.3 mg/lesion) | | | |
| Combination with | Melanoma | yes | PD |
| pembrolizumab | | | |
| (0.3 mg/lesion) | | | |
| Combination with | Pancreatic | no | SD |
| pembrolizumab | | | |
| (0.3 mg/lesion) | | | |
| Combination with | Melanoma | yes | PR |
| pembrolizumab | | | |
| (0.5 mg/lesion) | | | |
| Combination with | Leiomyosarcoma | no | SD |
| pembrolizumab | | | |
| (0.5 mg/lesion) | | | |
| Combination with | Adenoid cystic | no | NE |
| pembrolizumab | carcinoma | | |
| (0.5 mg/lesion) | | | |
| Combination with | Melanoma | yes | PD |
| pembrolizumab | | | |
| (0.5 mg/lesion) | | | |

Abbreviations:
RECIST Response Evaluation Criteria in Solid Tumors;
CR complete response;
PR partial response;
SD stable disease;
PD progressive disease;
NE not evaluable

Out of the 23 patients dosed, four patients continue with treatment with compound 1 and three 5 patients received compound 1 for longer than 24 weeks.

| | Tumor | Duration on |
|---|---|---|
| Study Drug | type | compound 1 (days)* |
| Monotherapy | Melanoma | 316 |
| (0.3 mg/lesion) | | |
| Monotherapy | Pancreatic | 22 |
| (0.3 mg/lesion) | | |
| Monotherapy | Breast cancer | 1 |
| (0.3 mg/lesion) | | |
| Monotherapy | Melanoma | 22 |
| (0.5 mg/lesion | | |
| Monotherapy | Melanoma | 208 |
| (0.5 mg/lesion) | | |
| Monotherapy | Squamous cell | 21 |
| (0.5 mg/lesion) | | |
| Monotherapy | Head and Neck | 106 |
| (0.5 mg/lesion) | Squamous Cell | |
| Monotherapy | Pancreatic | 1 |
| (0.5 mg/lesion) | | |
| Monotherapy | Colon Mixed | 26 |
| (0.5 mg/lesion) | Adenoneuroendocrine | |
| Combination with | Basel Cell | 49 |
| pembrolizumab | Carcinoma | |
| (0.3 mg/lesion) | | |
| Combination with | Melanoma | 71 |
| pembrolizumab | | |
| (0.3 mg/lesion) | | |
| Combination with | Pancreatic | 71 |
| pembrolizumab | | |
| (0.3 mg/lesion) | | |
| Combination with | Melanoma (and | 231 (ongoing) |
| pembrolizumab | Cutaneous | |
| (0.5 mg/lesion) | Squamous Cell) | |
| Combination with | Leiomyosarcoma | 116 |
| pembrolizumab | | |
| (0.5 mg/lesion) | | |
| Combination with | Adenoid cystic | 1 |
| pembrolizumab | carcinoma | |
| (0.5 mg/lesion) | | |
| Combination with | Melanoma | 51 |
| pembrolizumab | | |
| (0.5 mg/lesion) | | |
| Combination with | Melanoma | 50 |
| pembrolizumab | | |
| (0.5 mg/lesion) | | |
| Combination with | Head and Neck | 49 |
| pembrolizumab | Squamous Cell | |
| (0.5 mg/lesion) | | |
| Combination with | Mesothelioma | 32 (ongoing) |
| pembrolizumab | | |
| (0.5 mg/lesion) | | |
| Combination with | Head and Neck | 1 (ongoing) |
| pembrolizumab | Squamous Cell | |
| (0.5 mg/lesion) | | |
| Combination with | Liposarcoma | 1 |
| pembrolizumab | | |
| (0.5 mg/lesion) | | |
| Combination with | Sarcoma | 1 (ongoing) |
| pembrolizumab | | |
| (0.5 mg/lesion) | | |
| Combination with | Colon | 1 |
| pembrolizumab | | |
| (0.5 mg/lesion) | | |

*Duration is calculated from the time of first administration to the time of the last administration. If the subject only received one administration the duration of treatment is one day.

### PK Measurements

Resiquimod systemic concentration was determined following IT administration of compound 1 at a dose of 0.3 and 0.5 mg per lesion. The updated results are based on the data obtained in 17 patients. There were no significant differences between the two different doses. The mean systemic half-life of resiquimod released from the intratumoral administered depot was approximately 9 days. Cₘₐₓ values were generally below 150 μg/mL and well below levels reported in the literature to be associated with cytokine release syndrome (˜4000 μg/mL). There was no observed interaction with pembrolizumab.

### Biomarker Measurements

Sustained immune gene activation following repeat administration of compound 1 was observed and no difference was seen between the 0.3 mg/lesion and 0.5 mg/lesion doses. Specifically, there was a 7.4 times increase in IRF7 gene expression, 37.4 times increase in CXCL 10 gene expression, 4.7 times increase in STAT1 gene expression and 12.8 times increase in MX1 gene expression when comparing levels from cycle 1 day 8 to pre-treatment screening values in injected tumor lesions indicating sustained TLR7/8 pathway and interferon pathway gene activation. Sustained upregulation of expression of HLA genes was also observed with a 3.3 times increase in HLA-B gene expression and 3.7 fold increase in HLA-C gene expression when comparing cycle 1 day 8 levels to pre-treatment screening values in injected tumor lesions. Upregulation of gene signatures for cytotoxic immune cells (CD8 T cell and NK cell) but not regulatory T cells was also observed in the injected lesions. Gene expression remained elevated after a second administration, indicating sustained immune activation. Increases in plasma CXCL10 and TNF-alpha protein levels were found to be statistically significant at cycle 1 day 8 compared to pre-dose plasma concentrations documenting sustained elevated immune activation for at least one week after treatment. In one patient, a paired biopsy sample of a non-injected lesion demonstrated a 2.8 times increase in CD8 positive T cells, 16.8 fold increase in CD68 positive macrophages and no increase in FoxP3 positive regulatory T cells when comparing cycle 1 day 8 to cycle 2 day 8.

### CONCLUSION

Compound 1 was administered at 0.3 mg and 0.5 mg per lesion both as monotherapy and in combination with pembrolizumab to patients with solid tumors. Compound 1 is well tolerated with the majority of adverse events of Grade 1 or 2 per CTCAE v. 5 (Common Terminology Criteria for Adverse Events). There was one DLT observed of a Grade 3 injection site reaction in the 0.5 mg/lesion of compound 1 in combination with pembrolizumab. Compound 1 demonstrates clinical activity both as monotherapy and in combination with pembrolizumab in heavily treated advanced solid tumor patients with one abscopal (non-injected lesion) effect observed. PK and biomarker data suggest no difference between the two tested doses of compound 1 with low systemic concentrations of resiquimod and sustained activation of immune markers. Based on this data, the recommended phase 2 dose is 0.5 mg/lesion of compound 1, both as monotherapy and in combination with pembrolizumab.

### Abbreviations

AE Adverse event
Cₘₐₓ Maximum concentration
CR Complete response
CTCAE Common Terminology Criteria for Adverse Events
DLT Dose-limiting toxicities
ECOG Eastern Cooperative Oncology Group
EMA European Medicines Agency
FDA United States Food and Drug Administration
HPV Human papillomavirus
IT Intratumoral
mg Milligram
NE not evaluable
PD Pharmacodynamic/s
PD-1 Programmed cell death protein 1
PD-L1 Programmed cell death ligand 1
PEG Poly(ethylene glycol)
PR Partial response
Q3W Every 3 weeks
RECIST Response Evaluation Criteria in Solid Tumors
RP2D Recommended Phase 2 Dose
SAE Serious adverse event
SCCHN Squamous cell carcinoma of the head and neck
SRC Safety Review Committee
TEAE Treatment emergent adverse events
TLR Toll-like receptor
TNBC Triple-negative breast cancer

1-95. (canceled)

96. A method for the treatment of cancer, wherein the method comprises administering to a patient in need thereof a pharmaceutically effective amount of a TLR7/8 agonist conjugate or a pharmaceutically acceptable salt thereof, wherein the TLR7/8 agonist conjugate comprises one or more TLR7/8 agonist moieties reversibly conjugated to a polymeric moiety, wherein the cancer is a solid tumor and wherein the TLR7/8 agonist conjugate is administered via intratumoral administration in a dose ranging from 0.3 mg to 3 mg of TLR7/8 agonist per tumor.

97. The method of claim 96, wherein the TLR7/8 agonist conjugate is administered in a dose of 0.5 mg of TLR7/8 agonist per tumor.

98. The method of claim 96, wherein the dose is administered in a volume ranging from 0.1 ml to 2 ml.

99. The method of claim 96, wherein the dose is administered in a volume of 0.5 ml.

100. The method of claim 96, wherein the dose is administered in a volume of 1 ml.

101. The method of claim 96, wherein the dose is administered in a volume of 0.25 ml.

102. The method of claim 96, wherein the TLR7/8 agonist conjugate or a pharmaceutically acceptable salt thereof is provided as a pharmaceutical formulation comprising the TLR7/8 agonist conjugate or a pharmaceutically acceptable salt thereof and one or more excipients.

103. The method of claim 96, wherein the TLR7/8 agonist conjugate is of formula (A-3) 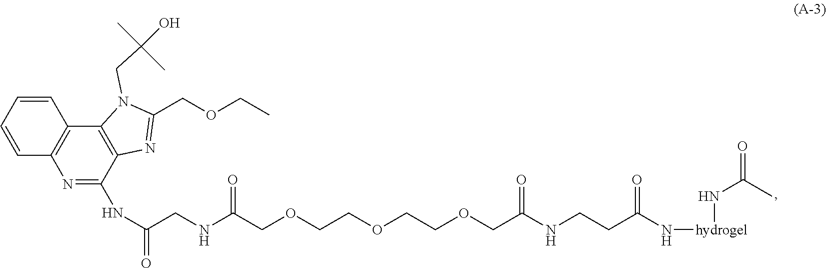
wherein “hydrogel” is a hydrogel comprising backbone moieties of formula (A-4): 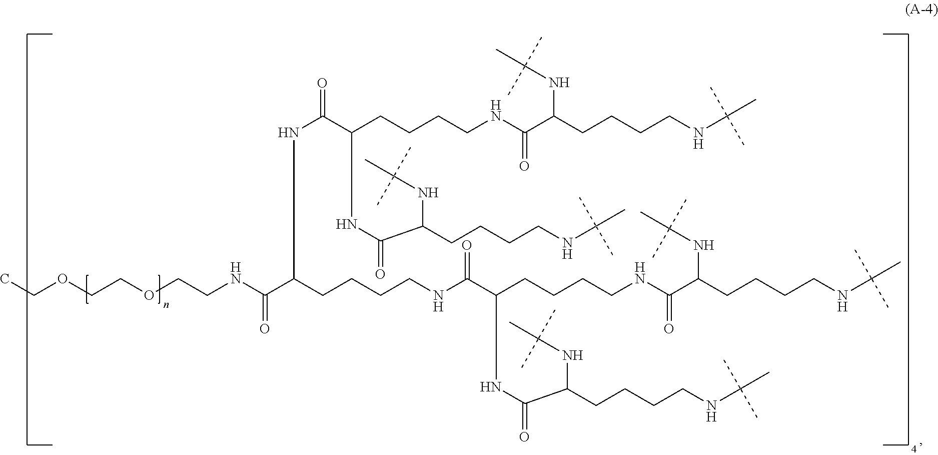
wherein n ranges from approx. 25 to 29 and dashed lines indicate attachment to either a moiety 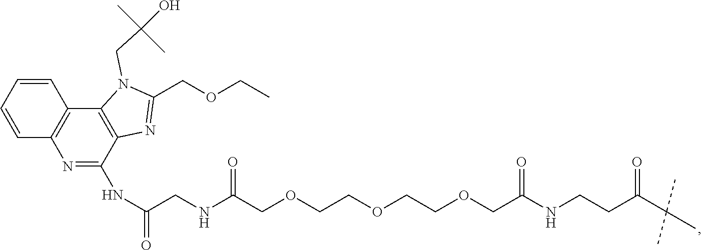
to a moiety 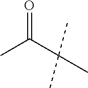
or to a crosslinker of formula (A-5): 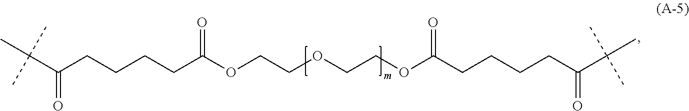
wherein m ranges from approx. 41 to 45 and the dashed lines in (A-5) indicate attachment to a backbone moiety of formula (A-4).

104. The method of claim 96, wherein the TLR7/8 agonist conjugate is of formula (Ai-6): 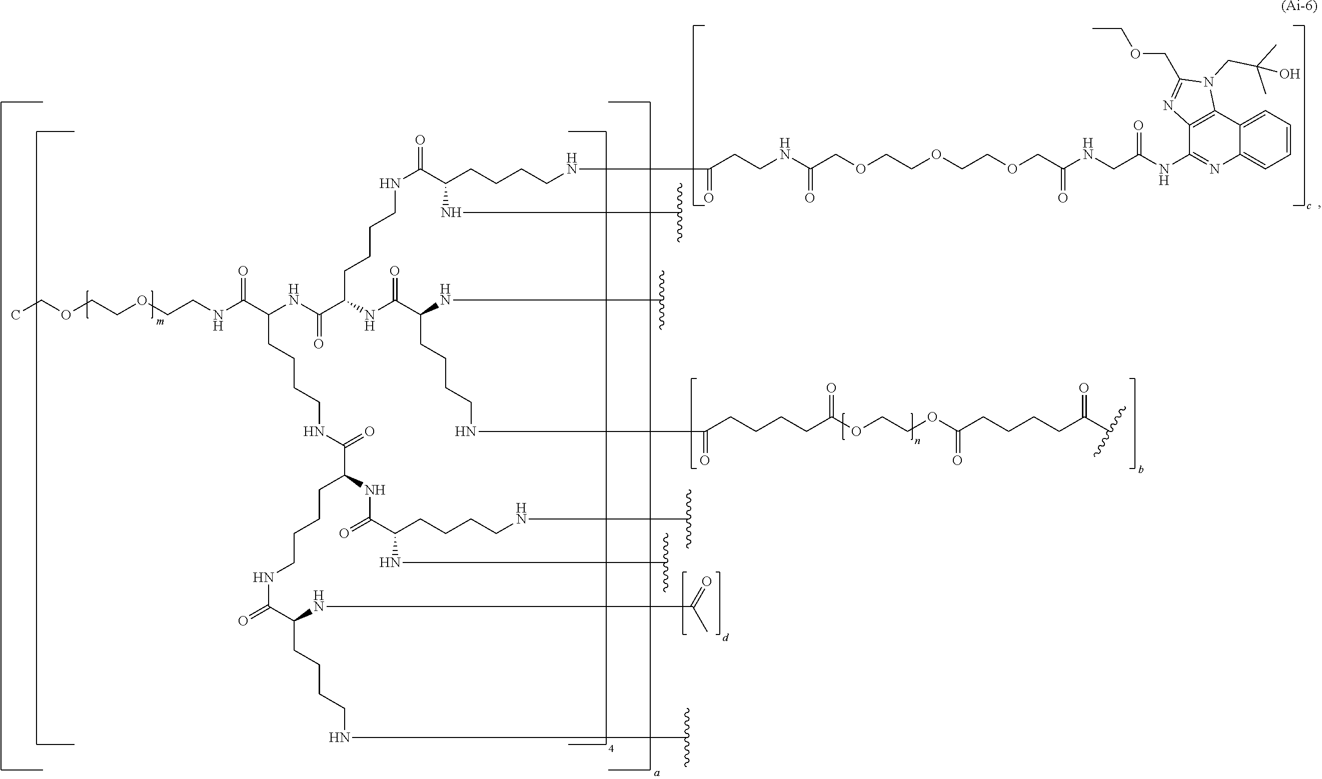
wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and the ratio of moieties “a”:“b”:“c”:“d” in the conjugate is approx. 1:13:c:d with c+d=6.

105. The method of claim 104, wherein the ratio of moieties “a”:“b”:“c”:“d” in the conjugate is approx. 1:13:3:3.

106. The method of claim 104, wherein the ratio of moieties “a”:“b”:“c”:“d” in the conjugate is approx. 1:13:2:4.

107. The method of claim 104, wherein the ratio of moieties “a”:“b”:“c”:“d” in the conjugate is approx. 1:13:4:2.

108. The method of claim 96, wherein the TLR7/8 agonist conjugate is provided in a vial.

109. The method of claim 96, wherein the TLR7/8 agonist conjugate is provided in a dual-chamber cartridge.

110. The method of claim 96, wherein the intratumoral injection is performed using a fanning technique.

111. The method of claim 96, wherein the dose is administered every two to four weeks.

112. The method of claim 96, wherein the dose is administered every three weeks.

113. The method of claim 96, wherein the solid tumor is selected from the group consisting of lip and oral cavity cancer, oral cancer, liver cancer/hepatocellular cancer, primary liver cancer, lung cancer, lymphoma, malignant mesothelioma, malignant thymoma, skin cancer, intraocular melanoma, metastasic squamous neck cancer with occult primary, childhood multiple endocrine neoplasia syndrome, mycosis fungoides, nasal cavity and paranasal sinus cancer, nasopharyngeal cancer, neuroblastoma, oropharyngeal cancer, ovarian cancer, pancreatic cancer, parathyroid cancer, pheochromocytoma, pituitary tumor, adrenocortical carcinoma, AIDS-related malignancies, anal cancer, bile duct cancer, bladder cancer, brain and nervous system cancer, breast cancer, bronchial adenoma/carcinoid, gastrointestinal carcinoid tumor, carcinoma, colorectal cancer, endometrial cancer, esophageal cancer, extracranial germ cell tumor, extragonadal germ cell tumor, extrahepatic bile duct cancer, gallbladder cancer, gastric (stomach) cancer, gestational trophoblastic tumor, head and neck cancer, hypopharyngeal cancer, islet cell carcinoma (endocrine pancreas), kidney cancer/renal cell cancer, laryngeal cancer, pleuropulmonary blastoma, prostate cancer, transitional cell cancer of the renal pelvis and ureter, retinoblastoma, salivary gland cancer, sarcoma, Sezary syndrome, small intestine cancer, genitourinary cancer, malignant thymoma, thyroid cancer, Wilms' tumor and cholangiocarcinoma.

114. The method of claim 96, wherein the solid tumor is selected from the group consisting of pancreatic cancer, prostate cancer, melanoma, SCCHN, cutaneous squamous cell cancer (cSCC) and cervical cancer.

115. The method of claim 96, wherein the TLR7/8 agonist conjugate is administered to one tumor of a patient.

116. The method of claim 96, wherein the TLR7/8 agonist conjugate is administered to more than one tumor of a patient.

117. The method of claim 96, wherein administration of the TLR7/8 agonist conjugate to one tumor leads to an abscopal effect in one or more untreated tumors.

118. The method of claim 96, wherein the treatment is a cotreatment with an immune checkpoint inhibitor, which may be given prior to, together with or after administration of the TLR7/8 agonist conjugate.

119. The method of claim 96, wherein the treatment is a cotreatment with an inhibitor of a PD-1 or an inhibitor of PD-L1, which may be given prior to, together with or after administration of the TLR7/8 agonist conjugate.

120. The method of claim 96, wherein the treatment is a cotreatment with an inhibitor of PD-1, which may be given prior to, together with or after administration of the TLR7/8 agonist conjugate.

121. The method of claim 96, wherein the treatment is a cotreatment with pembrolizumab, which may be given prior to, together with or after administration of the TLR7/8 agonist conjugate.

122. The method of claim 121, wherein the pembrolizumab is administered via intravenous injection every three weeks.

123. A conjugate or a pharmaceutically acceptable salt thereof of formula (A-3) 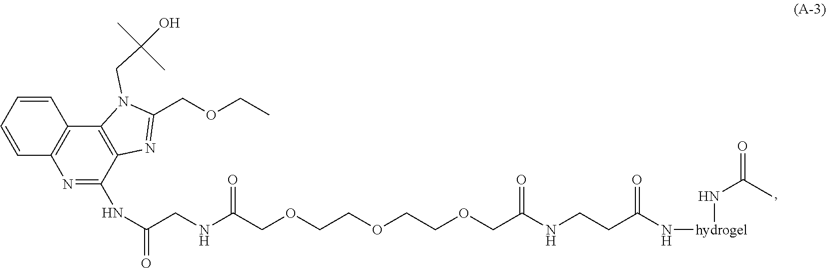
wherein “hydrogel” is a hydrogel comprising backbone moieties of formula (A-4): 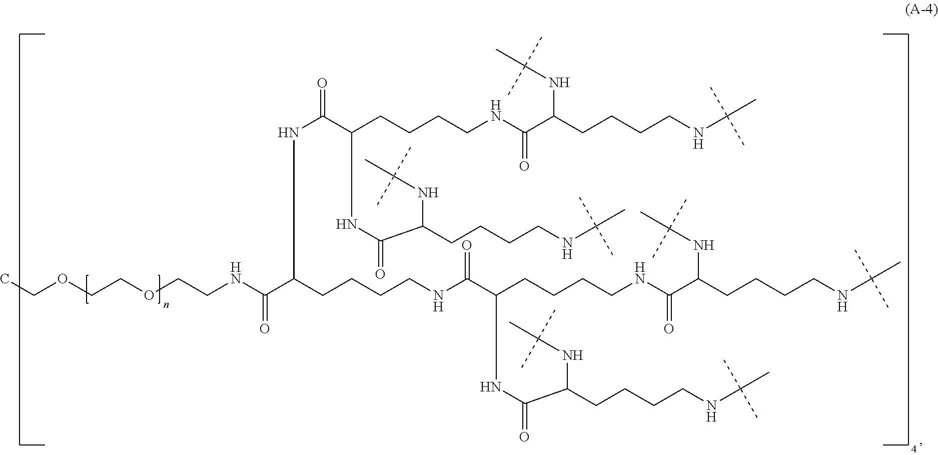
wherein n ranges from approx. 25 to 29 and dashed lines indicate attachment to either a moiety 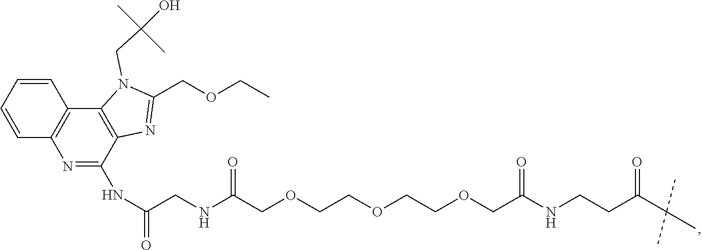
to a moiety 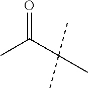
or to a crosslinker of formula (A-5): 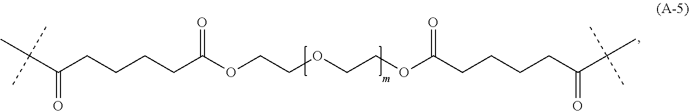
wherein m ranges from approx. 41 to 45 and the dashed lines in (A-5) indicate attachment to a backbone moiety of formula (A-4).

124. A conjugate or a pharmaceutically acceptable salt thereof of formula (Ai-6) 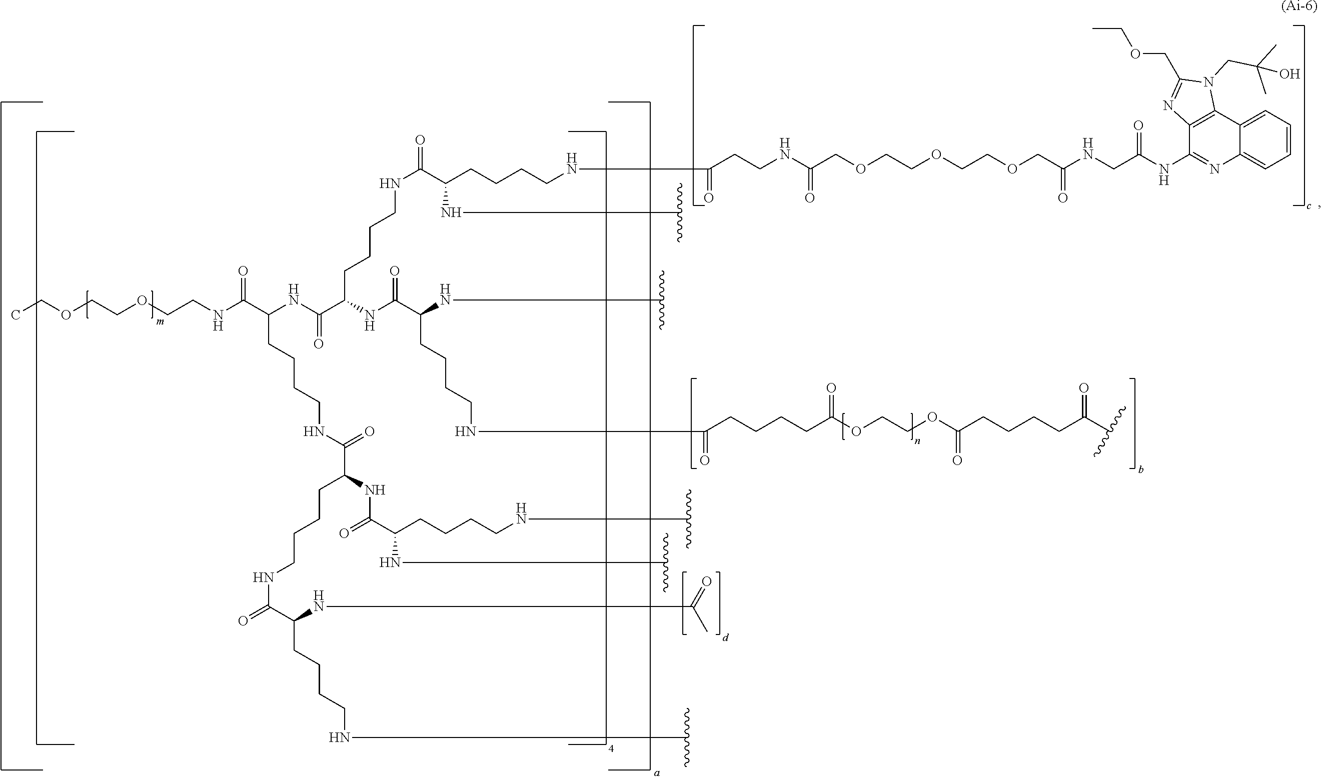
wherein
m ranges from approx. 25 to 29;
n ranges from approx. 41 to 45;
“a” denotes a backbone moiety, “b” denotes a crosslinker moiety, “c” denotes a reversibly conjugated resiquimod moiety and “d” denotes an acetamide moiety; and
the ratio of moieties “a”:“b”:“c”:“d” in the conjugate is approx. 1:13:c:d with c+d=6.

125. The conjugate or a pharmaceutically acceptable salt thereof of claim 124, wherein the ratio of moieties “a”:“b”:“c”:“d” in the conjugate is approx. 1:13:3:3.

126. The conjugate or a pharmaceutically acceptable salt thereof of claim 124, wherein the ratio of moieties “a”:“b”:“c”:“d” in the conjugate is approx. 1:13:2:4.

127. The conjugate or a pharmaceutically acceptable salt thereof of claim 124, wherein the ratio of moieties “a”:“b”:“c”:“d” in the conjugate is approx. 1:13:4:2.

128. A pharmaceutical formulation comprising one or more conjugate or a pharmaceutically acceptable salt thereof of claim 28 and one or more excipients.

129. A pharmaceutical formulation comprising one or more conjugate or a pharmaceutically acceptable salt thereof of claim 124 and one or more excipients.
